SEQUENCE LISTING (3 sequences)

SEQ ID NO: 1 - DNA, 17 bp, synthetic construct
  source 1..17: mol_type = other DNA
ccgctgcccaacacaag

SEQ ID NO: 2 - DNA, 24 bp, synthetic construct
  source 1..24: mol_type = other DNA
ccactaacgttcttttgcagacat

SEQ ID NO: 3 - DNA, 31 bp, synthetic construct
  source 1..31: mol_type = other DNA
  misc_feature 1: note = 5' Fluorescein tag
  misc_feature 31: note = 3' Iowa Black Fluorescent Quencher
  misc_feature 9..10: note = ZEN, or Internal Quencher, is located between the 9th and 10th bases from the 5' end of the probe.
agcctaccttgacaagcaatcagacactcaa

(19) United States
(12) Patent Application Publication
(10) Pub. No.: US 2026/0285903 A1
(43) Pub. Date: Sep. 24, 2026

(54) SYNTHESIZED BIOLOGICALLY ACTIVE COMPOUNDS AND METHODS OF MAKING AND USING

(71) Applicants: Georgia State University Research Foundation, Inc., Atlanta, GA (US); The United States Government as Represented by The Department of Veterans Affairs, Washington, DC (US)

(72) Inventors: Didier Merlin, Atlanta, GA (US); Chunhua Yang, Atlanta, GA (US)

(21) Appl. No.: 19/476,563

(22) PCT Filed: Apr. 16, 2024
(86) PCT No.: PCT/US2024/024749

§ 371 Date: Oct. 17, 2025

Related U.S. Application Data
(60) Provisional application No. 63/496,687, filed on Apr. 17, 2023.

(51) Int. Cl. C07K 5/02; A61K 38/00; A61K 45/06; A61P 29/00; A61P 31/14; A61P 35/00; C07K 5/093

(52) U.S. Cl. CPC C07K 5/0215; A61K 45/06; A61P 29/00; A61P 31/14; A61P 35/00; C07K 5/0819; A61K 38/00

(57) ABSTRACT

Compositions and formulations containing synthetic M13 analogs are disclosed. The M13 analogs and formulations have anti-inflammatory and antiviral properties, and are suitable for treating multiple types of inflammation and viral infections. Methods of treating, or reducing the risk of developing, one or more symptoms of a viral infection or a disease associated with a viral infection or disease associated with a viral infection in a subject. The methods include administering to the subject a therapeutically effective amount of the pharmaceutical formulation containing one or more synthetic M13 analogs. Also provided are methods of reducing an inflammatory response associated with a viral infection in a subject, by administering to the subject, a therapeutically effective amount of a pharmaceutical formulation containing M13 analogs.

### CROSS-REFERENCE TO RELATED APPLICATIONS

This application claims the benefit of and priority to U.S. Application No. 63/496,687 filed Apr. 17, 2023, the contents of which is incorporated by reference in its entirety.

### STATEMENT REGARDING FEDERALLY SPONSORED RESEARCH

This invention was made with government support under BX004476, and DK116306 awarded by the National Institutes of Health. The government has certain rights in the invention.

### REFERENCE TO SEQUENCE LISTING

The Sequence Listing submitted as an xml file named “GSURF_2022-030-02_PCT_ST26”, created on Apr. 16, 2024, and having a size of 5,069 bytes, is hereby incorporated by reference pursuant to 37 C.F.R. § 1.8345(c)(1)

### FIELD OF THE INVENTION

The disclosed invention is generally in the field of antiviral and anti-inflammatory agents and specifically in the area of compositions and methods for treatment of viral infections and inflammatory dysregulation.

### BACKGROUND OF THE INVENTION

Global health concern has motivated the exploration of natural products as lead compounds to develop new safe and effective drugs for treating symptoms of COVID-19 infection. Further, attempts to reduce inflammation associated viral infections have met with limited success.

Non-steroidal anti-inflammatory drugs (NSAIDS) have been used as the first-line drugs against inflammation. Besides blocking pro-inflammatory molecules, many anti-inflammatory drugs, also inhibit regulatory loops that release endogenous anti-inflammatory molecules. For example, NSAIDs reduce inflammation by blocking the enzymatic activity of cyclooxygenase, a key enzyme that catalyzes the conversion of arachidonic acid to prostaglandins and leukotrienes. Thus, NSAIDs reduce inflammation by preventing the synthesis of all prostaglandins. However, NSAIDs not only prevents the synthesis of proinflammatory prostaglandins, but also prevent the synthesis of anti-inflammatory prostaglandins. Hence, NSAIDs have limited success as they block endogenous anti-inflammatory response, which in some instances may prolong the inflammatory response.

Several natural products were found to render improved anti-inflammatory and antiviral properties than NSAIDS. However, these natural products are highly toxic and have poor biopharmaceutical properties such as poor water solubility and poor cell permeability.

Accordingly, there remains a need for more effective antivirals for treating viral infections, for example, infection by SARS-CoV-2 and variants thereof. There is also a need for more effective compounds and compositions for treating inflammation associated with inflammatory diseases/disorders and cancer.

An object of the invention is to provide compositions containing synthetic tripeptide conjugates with improved biopharmaceutical properties.

It is also an object of the invention to provide improved methods of treating viral infections or diseases associated with a viral infection.

It is another object of the invention to provide compositions and methods of use thereof, for treating inflammatory diseases/disorders and cancer.

### BRIEF SUMMARY OF THE INVENTION

M13 is a major phase II metabolite of the phenolic natural compound 6-shogaol. Synthetic M13 analogs (also referred herein as “compounds”) have been developed. The synthetic compounds disclosed herein are analogs of tripeptide conjugate of natural phenolic compounds The synthetic compounds disclosed herein have anti-inflammatory and antiviral properties and are suitable for use in, for example, the treatment of multiple types of inflammation and viral infections. In particular, these compounds are suitable for treating viral infections, such as SARS-CoV-2 infection. The disclosed compounds can also be used to treat a viral infection, a microbial infection, cancer, an inflammatory response, or a combination thereof, in a subject.

The compounds can have the structures of Formula I:

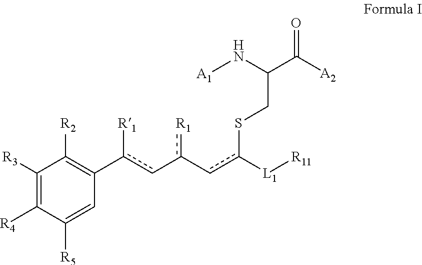

wherein: (i) [symbol] can be a single or double bond; (ii) A₁ and A₂ can be independently an amino acid residue; (iii) R₁ can be an oxygen, a hydroxyl, or —OR₆, and R₆ can be an unsubstituted linear, branched, or cyclic alkyl (such as C₁-C₆ alkyl); (iv) R′₁ can be a hydrogen, a hydroxyl, or —ORs, and R₈ can be an unsubstituted linear, branched, or cyclic alkyl (such as C₁-C₆ alkyl); and (v) R₂-R₅ can be independently a hydrogen, a hydroxyl, a halogen, an haloalkyl (e.g., —CF₃), or —OR₇, R₇ can be an unsubstituted linear, branched, or cyclic alkyl (such as C₁-C₆ alkyl), —C(═O)R₉, R₉ can be hydrogen, an unsubstituted linear, branched, or cyclic alkyl (such as C₁-C₆ alkyl), or —OR₁₀, and R₁₀ can be hydrogen or an unsubstituted linear, branched, or cyclic alkyl (such as C₁-C₆ alkyl); (vi) L₁ can be a single bond or —(CH₂)ₘ—, m is an integer from 1 to 8; (vii) R₁₁ can be a hydrogen, an unsubstituted or substituted linear C₁-C₁₀ alkyl, an unsubstituted or substituted branched C₃-C₁₀ alkyl, an unsubstituted or substituted C₃-C₁₀ cycloalkyl, or an unsubstituted or substituted C₅-C₁₂ aryl; and (viii) each substituent, when present, can be independently a halogen, a hydroxyl, an haloalkyl, an unsubstituted linear, branched, or cyclic alkyl (such as C₁-C₆ alkyl), an unsubstituted C₅-C₁₂ aryl, or —OR₁₂, R₁₂ can be an unsubstituted linear, branched, or cyclic alkyl (such as C₁-C₆ alkyl), and wherein the compound is not M13 having the following structure:

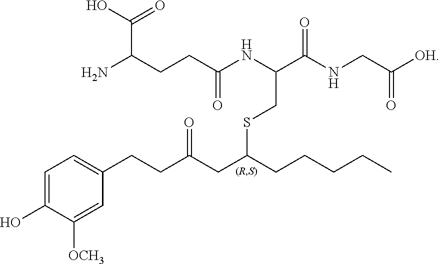 [SMILES: CCCCCC(CC(=O)CCc1ccc(O)c(OC)c1)SCC(NC(=O)CCC(N)C(=O)O)C(=O)NCC(=O)O]

In some forms, the compound can have the structures of Formula II:

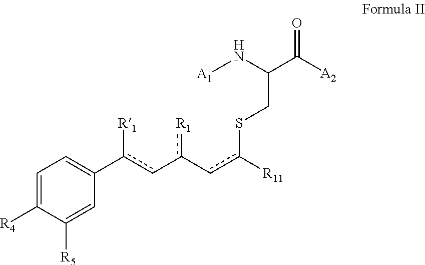

wherein: (i) [symbol] can be a single or double bond; (ii) A₁ and A₂ can be independently an amino acid residue; (iii) R₁ can be an oxygen, a hydroxyl, or —OR₆, and R₆ can be an unsubstituted linear, branched, or cyclic C₁-C₆ alkyl; (iv) R′₁ can be a hydrogen, a hydroxyl, or —ORs, and R₈ can be an unsubstituted linear, branched, or cyclic C₁-C₆ alkyl; (v) R₄ and R₅ can be independently a hydrogen, a hydroxyl, or —OR₇, R₇ can be an unsubstituted linear, branched, or cyclic C₁-C₆ alkyl; (vi) R₁₁ can be an unsubstituted linear C₅-C₉ alkyl, an unsubstituted branched C₃-C₅ alkyl, an unsubstituted C₃-C₈ cycloalkyl, an unsubstituted C₅-C₁₂ aryl, or a C₅-C₁₂ aryl substituted with a halogen, a hydroxyl, a haloalkyl, or —OR₁₂, R₁₂ can be an unsubstituted linear, branched, or cyclic C₁-C₆ alkyl, or a combination thereof.

In some forms, the compound can have the structures of Formula III:

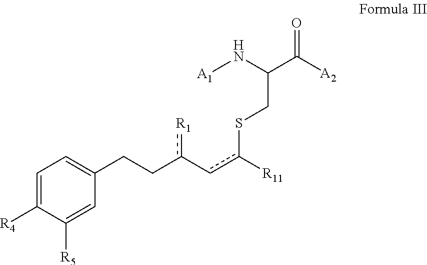

wherein: (i) [symbol] can be a single or double bond; (ii) A₁ and A₂ can be [symbol] independently an amino acid residue; (iii) R₁ can be an oxygen, a hydroxyl, or —OR₆, and R₆ can be an unsubstituted C₁-C₆ alkyl; (iv) R₄ can be a hydrogen or hydroxyl; (v) R₅ can be a hydrogen, a hydroxyl, or —OR₇, and R₇ can be an unsubstituted C₁-C₆ alkyl; and (vi) R₁₁ can be an unsubstituted branched C₃-C₆ alkyl, an unsubstituted C₃-C₆ cycloalkyl, an unsubstituted phenyl, or a phenyl substituted with a halogen.

In some forms, the compound can have the structures of Formula IV:

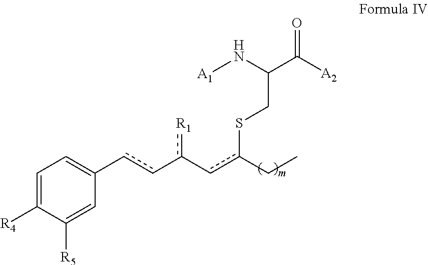

wherein: (i) [symbol] can be a single or double bond; (ii) m can be an integer from [symbol] 4 to 8; (iii) A₁ and A₂ can be independently an amino acid residue; (iv) R₁ can be an oxygen, a hydroxyl, or —OR₆, and R₆ can be an unsubstituted linear, branched, or cyclic C₁-C₆ alkyl; (v) R₄ can be a hydrogen or hydroxyl; and (vi) R₈ can be a hydrogen, a hydroxyl, or —OR₇, and R₇ can be an unsubstituted linear, branched, or cyclic C₁-C₆ alkyl.

In some forms, the compound can have the structures of Formula V:

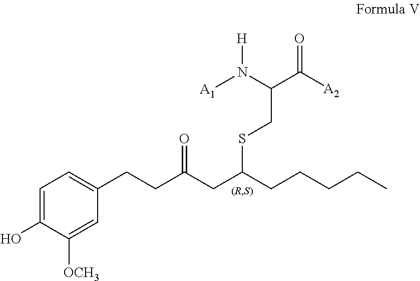

wherein A₁ and A₂ can be independently an amino acid residue.

For Formulae I-V, A₁ can be any suitable amino acid residue (natural or synthetic), as long as the pH of the amino acid forming A₁ is similar to the pH of glutamic acid; and A₂ can be any suitable amino acid residue (natural or synthetic), as long as the pH of the amino acid forming A₂ is similar to the pH of glycine. It is important that the amino acids forming A₁ and A₂ have similar pH to glutamic acid and glycine respectively to mimic the structure of glutathione (GSH). GSH neutralization is one of the primary prevention mechanisms against reactive oxygen species and electrophiles in living organisms.

In some forms, for any of Formulae I-V described herein, A₁ can be a natural amino acid residue (such as glutamic acid residue or an aspartic acid residue), or a synthetic amino acid residue where the amino acid forming A₁ can be a pH similar to glutamic acid; and A₂ can be a natural amino acid residue (such as an alanine residue, a phenylalanine residue, a leucine residue, a methionine residue, a proline residue, a valine residue, a tryptophan residue, a tyrosine residue, or a glycine residue), or a synthetic amino acid residue where the amino acid forming A₂ can be a pH similar to glycine.

In some forms, for any of Formulae I-V described herein, A₁ can be a glutamic acid residue or an aspartic acid residue, or a synthetic derivative thereof; and A₂ can be an alanine residue, a phenylalanine residue, a leucine residue, a methionine residue, a proline residue, a valine residue, a tryptophan residue, a tyrosine residue, or a glycine residue, or a synthetic derivative thereof.

In some forms, for any of Formulae I-V described herein, A₁ can be a glutamic acid residue or an aspartic acid residue, or a synthetic derivative thereof; and A₂ can be a leucine residue, a methionine residue, a valine residue, a tryptophan residue, an alanine residue, a phenylalanine residue, or a tyrosine residue, or a synthetic derivative thereof.

In some forms, for any of Formulae I-V described herein, A₁ can be an alpha-glutamic acid residue, a gamma-glutamic acid residue, an alpha-aspartic acid residue, or a beta-aspartic acid residue; and A₂ can be an alanine residue, a phenylalanine residue, a leucine residue, a methionine residue, a proline residue, a valine residue, a tryptophan residue, a tyrosine residue, or a glycine residue, or a synthetic derivative thereof.

Pharmaceutical formulations containing one or more of the synthetic compounds and/or M13 (a metabolite of 6-shogaol) are also disclosed. The structure of M13 is shown below. These pharmaceutical formulations are suitable for oral, intravenous, and/or nasal administration to a subject in need thereof, for treating viral infections, such as respiratory viral infection, for example, SARS-CoV-2 infection.

In some forms, the one or more compounds in the formulation are collectively in an amount effective, when administered to a subject, to reduce the expression in the subject of one or more pro-inflammatory cytokines and/or chemokines selected from the group consisting of TNFα, 1L6, and IL1-p. In some forms, the one or more compounds in the formulation are each individually in an amount effective, when administered to a subject, to reduce the expression in the subject of one or more pro-inflammatory cytokines and/or chemokines selected from the group consisting of TNFα, IL6, and IL1-$.

In some forms, the one or more compounds in the formulation are collectively in an amount effective, when administered to a subject, to increase the expression of IL-10 in the subject. In some forms, the one or more compounds in the formulation are each individually in an amount effective, when administered to a subject, to increase the expression of IL-10 in the subject.

In some forms, the one or more compounds in the formulation are collectively in an amount effective, when administered to a subject, to reduce the expression in the subject of one or more kinases selected from the group consisting of PI3K, SPHK2, CDK6, TRKB, C-MER, EPHB2, RIPK4, RIPK5, and RIPK4. In some forms, the one or more compounds in the formulation are each individually in an amount effective, when administered to a subject, to reduce the expression in the subject of one or more kinases selected from the group consisting of PI3K, SPHK2, CDK6, TRKB, C-MER, EPHB2, RIPK4, RIPK5, and RIPK4.

In some forms, the one or more compounds in the formulation are collectively in an amount effective, when administered to a subject, to treat a viral infection, a microbial infection, cancer, an inflammatory response, or a combination thereof, in the subject. In some forms, the one or more compounds in the formulation are each individually in an amount effective, when administered to a subject, to treat a viral infection, a microbial infection, cancer, an inflammatory response, or a combination thereof, in the subject.

In some forms, the one or more compounds in the formulation are collectively in an amount effective, when administered to a subject, to ameliorate one or more symptoms of the viral infection, the microbial infection, the cancer, the inflammatory response, or of a combination thereof, in the subject. In some forms, the one or more compounds in the formulation are each individually in an amount effective, when administered to a subject, to ameliorate one or more symptoms of the viral infection, the microbial infection, the cancer, the inflammatory response, or of a combination thereof, in the subject.

In some forms, the one or more compounds in the formulation are collectively in an amount effective, when administered to a subject, to ameliorate one or more symptoms of a disease associated with the viral infection, the microbial infection, the cancer, the inflammatory response, or a combination thereof, in the subject. In some forms, the one or more compounds in the formulation are each individually in an amount effective, when administered to a subject, to ameliorate one or more symptoms of a disease associated with the viral infection, the microbial infection, the cancer, the inflammatory response, or a combination thereof, in the subject.

In some forms, the pharmaceutical formulation can further include one or more additional active agents. In some forms, the one or more active agents are selected from the group consisting of a therapeutic agent, a prophylactic agent, a diagnostic agent, an anti-inflammatory agent, and an adjuvant.

In some forms, the one or more compounds in the pharmaceutical formulation are collectively at a concentration of at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %.

In some forms, the one or more compounds in the pharmaceutical formulation are each individually at a concentration of at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %.

In some forms, the pharmaceutical formulation includes M13 and one or more pharmaceutically acceptable carriers and/or excipients. In some forms, the M13 in the formulation is in an amount effective, when administered to a subject, to treat a viral infection, a microbial infection, cancer, or a combination thereof, in the subject.

In some forms, the pharmaceutical formulation further includes one or more additional active agents. In some forms, the one or more active agents are selected from the group consisting of a therapeutic agent, a prophylactic agent, a diagnostic agent, an anti-inflammatory agent, and an adjuvant.

In some forms, the M13 in the formulation is in an amount effective, when administered to a subject, to reduce the expression in the subject of one or more pro-inflammatory cytokines and/or chemokines selected from the group comprising TNFα, IL6, and IL1-R. In some forms, the M13 in the formulation is in an amount effective, when administered to a subject, to increase the expression of IL-10 in the subject.

In some forms, the M13 in the formulation is in an amount effective, when administered to a subject, to reduce the expression in the subject of one or more kinases selected from the group comprising PI3K, SPHK2, CDK6, TRKB, C-MER, EPHB2, RIPK4, RIPK5, and RIPK4.

In some forms, the M13 in the pharmaceutical formulation is at a concentration of at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %.

Also disclosed are methods of treating a subject. In some forms, the method comprises administering to the subject any one of the disclosed pharmaceutical formulations. In some forms, the one or more compounds in the formulation can be collectively in an amount effective to treat a viral infection, a microbial infection, cancer, an inflammatory response, or a combination thereof, in the subject. In some forms, the administering can be performed one or more times.

In some forms, the collective dosage of the compounds in the pharmaceutical formulation is from about 0.1 mg per kg to about 50 mg per kg, from about 0.5 mg to about 25 mg per kg, from about 1 mg to about 50 mg per kg of the subject. In some forms, the individual dosage of each of the compounds in the pharmaceutical formulation is from about 0.1 mg per kg to about 50 mg per kg, from about 0.5 mg to about 25 mg per kg, from about 1 mg to about 50 mg per kg of the subject.

In some forms, the method comprises administering to the subject a pharmaceutical formulation including M13. In some forms, the M13 in the formulation is in an amount effective to treat a viral infection, a microbial infection, cancer, or a combination thereof, in the subject. In some forms, the dosage of the M13 in the pharmaceutical formulation is from about 0.1 mg per kg to about 50 mg per kg, from about 0.5 mg to about 25 mg per kg, from about 1 mg to about 50 mg per kg of the subject.

In some forms, the subject has a viral infection. In some forms, the viral infection is an infection by an RNA virus. In some forms, the RNA virus is of a family selected from the group consisting of Flaviviridae, Orthomyxoviridae, Filoviridae, Coronaviridae, and Paramyxoviridae. In some forms, the viral infection is an infection by a coronavirus.

In some forms, the coronavirus is an alpha coronavirus, a beta coronavirus, a gamma coronavirus, or a delta coronavirus. In some forms, the coronavirus is selected from the group consisting of Human Coronavirus 229E (HCoV-229E), Human Coronavirus OC43 (HCoV-OC43), Human Coronavirus NL63 (HCoV-NL63), Human Coronavirus HKU1 (HCoV-HKU1), Middle East Respiratory Syndrome Coronavirus (MERS-CoV), Severe Acute Respiratory Syndrome Coronavirus 1 (SARS-CoV-1), and SARS-CoV-2.

In some forms, the coronavirus is a SARS-CoV-2 variant, wherein the SARS-CoV-2 variant is the alpha variant, beta variant, gamma variant, delta variant, epsilon variant, eta variant, iota variant, kappa variant, mu variant, omicron variant, zeta variant, 1.617.3 variant and/or lambda variant. In some forms, the SARS-CoV-2 variant is a sub-variant of the alpha variant, beta variant, gamma variant, delta variant, epsilon variant, eta variant, iota variant, kappa variant, mu variant, omicron variant, zeta variant, 1.617.3 variant and/or lambda variant.

In some forms, the subject has a microbial infection. In some forms, the microbial infection is caused by microorganisms selected from the group consisting of a bacterium, a virus, a protozoan, and a fungus. In some forms, the subject has cancer. In some forms, the subject has an inflammatory response.

In some forms, the pharmaceutical formulation is administered by oral administration, intranasal administration, intratracheal administration, intramuscular administration, intravenous administration, intraperitoneal administration, or subcutaneous administration, or a combination thereof.

In some forms, the administration is repeated every 4 hours, every 6 hours, every 12 hours, or every day, optionally for a time period from 1 day to 1 month, from 1 day to 2 weeks, from 1 day to 1 week, or from 1 day to 3 days.

In some forms, the subject is a human. In some forms, the subject is immunocompromised.

Also disclosed are methods of treating, or reducing the risk of developing, one or more symptoms of a viral infection or a disease associated with a viral infection in a subject. The methods include administering to the subject a therapeutically effective amount of the pharmaceutical formulation containing one or more synthetic M13 analog (s).

Typically, the amount of the synthetic M13 analog (s) in the pharmaceutical formulation is effective, when administered to the subject, to reduce viral replication by 50% or more 24 hours following administration. In some forms, the pharmaceutical formulation is effective, when administered to the subject, to deliver the synthetic M13 analog (s) at a dose from about from about 0.1 mg per kg to about 50 mg per kg, from about 0.5 mg to about 25 mg per kg, from about 1 mg to about 50 mg per kg, such as about 10 mg per kg of the subject. In some forms, the pharmaceutical formulation is administered to the subject every 4 hours, every 6 hours, every 12 hours, or every day, optionally for a time period from 1 day to 1 month, from 1 day to 2 weeks, from 1 day to 1 week, or from 1 day to 3 days, or any combination thereof following the detection of the virus in the subject. In some forms, the pharmaceutical composition is administered via oral administration, intranasal administration, intratracheal administration, intramuscular administration, intravenous administration, intraperitoneal administration, or subcutaneous administration, or a combination thereof.

In preferred forms, the subject is a human. In some forms, the subject is immunocompromised. In preferred forms, the pharmaceutical formulation is used to treat a subject infected by an RNA virus, preferably an RNA virus of the family Coronaviridae. More preferably, the pharmaceutical formulation is used to treat a subject infected by a coronavirus.

In some forms, the subject is infected with an alpha coronavirus, a beta coronavirus, a gamma coronavirus, or a delta coronavirus. In some forms, the subject is infected with Human Coronavirus 229E (HCoV-229E), Human Coronavirus OC43 (HCoV-OC43), Human Coronavirus NL63 (HCoV-NL63), Human Coronavirus HKU1 (HCoV-HKU1), Middle East Respiratory Syndrome Coronavirus (MERS-CoV), Severe Acute Respiratory Syndrome Coronavirus 1 (SARS-CoV-1), and SARS-CoV-2.

In preferred forms, the pharmaceutical formulation is used to treat a subject infected by a variant of SARS-CoV-2, for example, the alpha variant, beta variant, gamma variant, delta variant, epsilon variant, eta variant, iota variant, kappa variant, mu variant, omicron variant, zeta variant, 1.617.3 variant and/or lambda variant. In preferred forms, the pharmaceutical formulation is used to treat a subject infected by a sub-variant of the alpha variant, beta variant, gamma variant, delta variant, epsilon variant, eta variant, iota variant, kappa variant, mu variant, omicron variant, zeta variant, 1.617.3 variant and/or lambda variant.

In some forms, the pharmaceutical formulation is used to treat a subject having a disease associated with a viral infection, for example a coronavirus infection such as coronavirus-induced pneumonia, coronavirus-induced bronchitis, acute respiratory distress syndrome (ARDS), acute lung injury (ALI), multisystem inflammatory syndrome in children (MIS-C), and/or multisystem inflammatory syndrome in adults (MIS-A).

Methods of reducing an inflammatory response associated with a viral infection in a subject are also provided. The methods include administering to the subject, a therapeutically effective amount of the pharmaceutical formulation containing one or more synthetic M13 analog (s).

### BRIEF DESCRIPTION OF THE DRAWINGS

The accompanying drawings illustrate several embodiments of the disclosed method and compositions and together with the description, serve to explain the principles of the disclosed method and compositions.

FIGS. 1A and 1B show the lack of mutagenic potential of M13. In FIG. 1A, A1 refers to the Tester strain TA98, under one of three conditions: untreated, treated with positive control (i.e., 2NF: 2-nitrofluorene), or PBS; and A₂ refers to the Tester strain TA98 treated with M13 at concentrations of 0.3125 mg/plate, 0.625 mg/plate, 1.25 mg/plate, 2.5 mg/plate, or 5 mg/plate. FIG. 1B shows the colony average revertant numbers on the negative, PBS positive control (2NF) and M13-treated plates as determined by manual counting.

FIGS. 2A and 2B are bar graphs showing the targeted inhibitor effects of M13 on lipid kinases (FIG. 2A) and protein kinases (FIG. 2B).

FIG. 3 shows the percent inhibition and cytotoxicity of M13 (Sample 2) when added one hour prior to infection with SARS-COV-2.

FIG. 4 shows the percent inhibition and cytotoxicity of M13 (Sample 2) when added one hour following infection with SARS-COV-2.

FIG. 5 shows the percent inhibition and cytotoxicity of Remdesivir when added one hour prior to infection with SARS-COV-2.

FIG. 6 is a schematic of an exemplary chemical reaction for a representative compound MLY2.

FIGS. 7A-7C are a representative HPLC readout (FIG. 7A), Mass spectrum (FIG. 7B), and 1H-NMR spectrum (FIG. 7C) for the exemplary compound MLY2.

FIGS. 8A and 8B are bar graphs showing fold changes in the relative NF-κB-dependent luciferase activity of the 19 exemplary MLY compounds.

FIG. 9 is a line graph showing fold changes in the relative NF-xB-dependent luciferase activity of the exemplary compounds MLY #2 and MLY #8.

FIGS. 10A and 10B are bar graphs showing the expression of IL-6 (FIG. 10A) and TNF-α (FIG. 10B) in cell lysates as indicated by relative pixel density.

FIGS. 11A-11K show that M13 has excellent biopharmaceutical properties. (FIG. 11A is an HPLC profile of M13 in acidic PBS. FIG. 11B is a bar graph of the relative M13 content before and after incubation (n=3). FIG. 11C shows the relative content of M13 or 6-shogaol (determined by HPLC) before and after incubation with mouse intestinal microsomes (n=3). FIG. 11D is a FACS of cultured PBMCs with gating on viable CD4+ and CD8+ T cells in the solvent control (SC) and M13-treated groups with (+) or without (−) IL-2 addition. FIG. 11E shows the percentage of viable proliferating CD4+ T cells. FIGS. 11F and 11G are bar graphs showing M13-treated-to-SC group ratio of the percentage of proliferating viable T cells with or without IL-2 addition (n=3). FIGS. 11H-11K show colony average revertant numbers (FIGS. 11H and 11J), and mutational factors (FIGS. 11I and 11K) of the Ames test using S. typhimurium strain TA100 with (+S9) or without (−S9) metabolic activation (n=3). Negative control (NC), no treatment; PBS, vehicle control; NaN3, (1 μg/plate), positive control; 2AA, (2-aminoanthracene, 2 g/plate), positive control. (ns, not significant; *p<0.05, **p<0.01; p=probability value denoting statistical significance).

FIGS. 12A-12G show the in vitro anti-cancer effects of M13 in 2D and 3D cultured intestinal cells. FIG. 12A is a cell viability curves of cells treated with M13 for 24 hours (n=3). FIG. 12B shows the IC50 values (24 h incubation, n=3). FIG. 12C is an experimental outline of dosing on droplet-based 3D Caco-2/BBe cells. (+: treatment, −: no treatment) FIG. 12D shows representative morphologies (Bright-field images).

Negative control (NC): no treatment. Well diameter: 6.4 mm. FIG. 12E is a bar graph showing comparison of the volume changes of 3D Caco-2/BBe cells. FIG. 12F shows the survival rates of 3D Caco-2/BBe cells (n=4). FIG. 12G is a dose-response curve of 3D Caco-2/BBe cells based on ATP contents (extrapolated, n=4). Error bars represent one standard deviation between two groups or compared with NC (*p<0.05, **p<0.01, ***p<0.001).

FIGS. 13A-13D illustrate the efficacies of M13 or M13-NL treatment on AOM/DSS-induced colon tumors in mice. FIG. 13A is an experimental timeline for AOM/DSS-induced tumorigenesis in the mouse model. Mice were orally administered free M13 (5 mg/kg), NL (5 mg/kg), or M13-NL (5 mg/kg M13 loaded into 5 mg/kg lipid nanoparticles) every other day after day 42. No treatment mice were used as the wild-type (WT) control. FIGS. 13B-13F show comparison of the colon lengths (FIG. 13B), the total number of tumors (FIG. 13C), and the number of the tumor size distribution (FIG. 13D) (n=5). (ns, not significant; *p<0.05, **p<0.01, ***p<0.001, ****p<0.0001).

FIGS. 14A-141 shows analysis of M13-NL against colon tumors in AOM/DSS-induced colon cancer mice. FIGS. 14A-141 show TUNEL- (FIG. 14A), Ki-67 (FIG. 14B), Survivin- (FIG. 14C), EGFR- (FIG. 14D), BCL-x- (FIG. 14E), Dkk-1- (FIG. 14F), EpCAM- (FIG. 14G), CDC25A- (FIG. 14H), and RAD52-positive (FIG. 14I) cells or area were counted and averaged per crypt (n=5). (ns, not significant; *p<0.05, **p<0.01, ***p<0.001).

FIGS. 15A-15E shows the cancer prevention effect of M13-NL on the AOM-exposed IL10^{-/-} mice. FIG. 15A is a schematic of the Timeline for AOM-exposed CAC tumorigenesis in IL10^{-/-} mice. PBS, NL (25 g/mL), or M13-NL (25 μg/mL M13 loaded into 25 μg/mL NL) were given in drinking water from weeks 12 to 31. FIG. 15B is a line graph showing Lcn-2 levels of fecal samples from weeks 5 to 29 (n=5). FIG. 15C is a bar graph showing the comparison of the colon lengths among different groups (n=5 or 6) FIG. 15D is a dot plot showing comparison of the total number of tumors (n=5 or 6).

FIG. 15E is a bar graph showing assessment of the spleen-to-body weight ratio (n=5). (ns, not significant; *p<0.05, **p<0.01, ***p<0.001).

### DETAILED DESCRIPTION OF THE INVENTION

### I. Definitions

It is to be understood that the disclosed compounds, compositions, and methods are not limited to specific synthetic methods, specific analytical techniques, or to particular reagents unless otherwise specified, and, as such, may vary. It is also to be understood that the terminology used herein is for the purpose of describing particular forms and embodiments only and is not intended to be limiting.

The numerical ranges disclose individually each possible number that such a range could reasonably encompass, as well as any sub-ranges and combinations of sub-ranges encompassed therein. For example, in a given range carbon range of C₃-C₉, the range also discloses C₃, C₄, C₅, C₆, C₇, C₈, and C₉, as well as any subrange between these numbers (for example, C₄-C₆), and any possible combination of ranges possible between these values. In yet another example, a given temperature range may be from about 25° C. to 30° C., where the range also discloses temperatures that can be selected independently from about 25, 26, 27, 28, 29, and 30° C., as well as any range between these numbers (for example, 26 to 28° C.), and any possible combination of ranges between these values.

Use of the term “about” is intended to describe values either above or below the stated value, which the term “about” modifies, to be within a range of approximately +/−10%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise. ° C., where the range also discloses temperatures that can be selected independently from about 25, 26, 27, 28, 29, and 30° C., as well as any range between these numbers (for example, 26 to 28° C.), and any possible combination of ranges between these values.

Use of the term “about” is intended to describe values either above or below the stated value, which the term “about” modifies, to be within a range of approximately +/−10%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise.

“Analog” as relates to a given compound, refers to another compound that is structurally similar, functionally similar, or both, to the specified compound. Structural similarity can be determined using any criterion known in the art, such as the Tanimoto coefficient that provides a quantitative measure of similarity between two compounds based on their molecular descriptors. Preferably, the molecular descriptors are 2D properties such as fingerprints, topological indices, and maximum common substructures, or 3D properties such as overall shape, and molecular fields. Tanimoto coefficients range between zero and one, inclusive, for dissimilar and identical pairs of molecules, respectively. A compound can be considered an analog of a specified compound, if it has a Tanimoto coefficient with the specified compound between 0.5 and 1.0, inclusive, preferably between 0.7 and 1.0, inclusive, most preferably between 0.85 and 1.0, inclusive. A compound is functionally similar to a specified, if it induces the same pharmacological effect, physiological effect, or both, as the specified compound. “Analog” can also refer to a modification including, but not limited to, hydrolysis, reduction, or oxidation products, of the disclosed compounds. Hydrolysis, reduction, and oxidation reactions are known in the art.

### II. Compositions

Synthetic M13 analogs (also referred herein as “synthetic compounds”) have been developed. The synthetic compounds disclosed herein have anti-inflammatory and antiviral properties and should be suitable for use in the treatment of multiple types of inflammation and viral infections. In particular, these compounds are suitable for treating respiratory viral infections, such as SARS-CoV-2 infection. In some forms, the synthetic compounds have greater anti-inflammatory and anti-viral properties than M13.

Pharmaceutical formulations containing the synthetic compounds, and M13 (a metabolite of 6-shogaol) are also disclosed. The structure of M13 is shown below. These pharmaceutical formulations are suitable for oral, intravenous, and/or nasal administration to a subject in need thereof, for treating viral infections, such as respiratory viral infection, for example, SARS-CoV-2 infection.

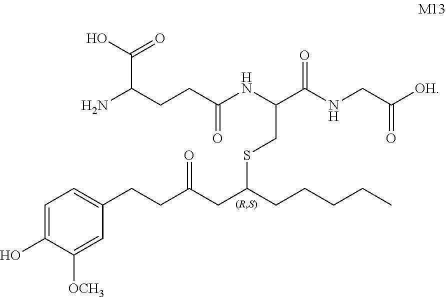 [SMILES: CCCCCC(CC(=O)CCc1ccc(O)c(OC)c1)SCC(NC(=O)CCC(N)C(=O)O)C(=O)NCC(=O)O]

### A. Compounds

### 1. Compound Structures

The synthetic compounds can have the structures of Formula I:

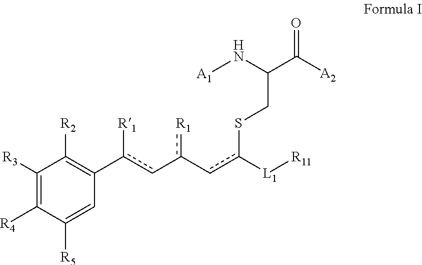

wherein: (i) [symbol] can be a single or double bond; (ii) A₁ and A₂ can be independently an amino acid residue; (iii) R₁ can be an oxygen, a hydroxyl, or —OR₆, and R₆ is an unsubstituted linear, branched, or cyclic alkyl (such as C₁-C₆ alkyl); (iv) R′₁ can be a hydrogen, a hydroxyl, or —ORs, and R₈ can be an unsubstituted linear, branched, or cyclic alkyl (such as C₁-C₆ alkyl); and (v) R₂-R₅ can be independently a hydrogen, a hydroxyl, a halogen, an haloalkyl (e.g., —CF₃), or —OR₇, R₇ can be an unsubstituted linear, branched, or cyclic alkyl (such as C₁-C₆ alkyl), —C(═O)R₉, R₉ can be hydrogen, an unsubstituted linear, branched, or cyclic alkyl (such as C₁-C₆ alkyl), or —OR₁₀, and R₁₀ can be hydrogen or an unsubstituted linear, branched, or cyclic alkyl (such as C₁-C₆ alkyl); (vi) L₁ can be a single bond or —(CH₂)ₘ—, m is an integer from 1 to 8; (vii) R₁₁ can be a hydrogen, an unsubstituted or substituted linear C₁-C₁₀ alkyl, an unsubstituted or substituted branched C₃-C₁₀ alkyl, an unsubstituted or substituted C₃-C₁₀ cycloalkyl, or an unsubstituted or substituted C₅-C₁₂ aryl; and (viii) each substituent, when present, can be independently a halogen, a hydroxyl, an haloalkyl, an unsubstituted linear, branched, or cyclic alkyl (such as C₁-C₆ alkyl), an unsubstituted C₅-C₁₂ aryl, or —OR₁₂, R₁₂ can be an unsubstituted linear, branched, or cyclic alkyl (such as C₁-C₆ alkyl), and wherein the compound is not M13 having the following structure:

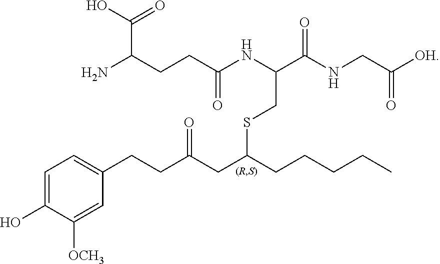 [SMILES: CCCCCC(CC(=O)CCc1ccc(O)c(OC)c1)SCC(NC(=O)CCC(N)C(=O)O)C(=O)NCC(=O)O]

In some forms, the synthetic compound can have the structure of Formula II:

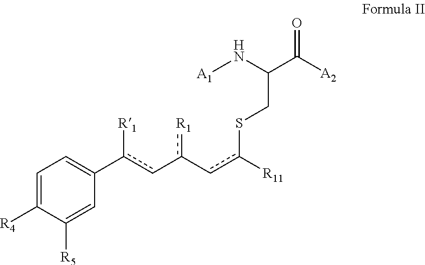

wherein: (i) [symbol] can be a single or double bond; (ii) A₁ and A₂ can be independently an amino acid residue; (iii) R₁ can be an oxygen, a hydroxyl, or —OR₆, and R₆ can be an unsubstituted linear, branched, or cyclic C₁-C₆ alkyl; (iv) R′₁ can be a hydrogen, a hydroxyl, or —OR₈, and R₈ can be an unsubstituted linear, branched, or cyclic C₁-C₆ alkyl; (v) R₄ and R₈ can be independently a hydrogen, a hydroxyl, or —OR₇, R₇ can be an unsubstituted linear, branched, or cyclic C₁-C₆ alkyl; (vi) R₁₁ can be an unsubstituted linear C₅-C₉ alkyl, an unsubstituted branched C₃-C₈ alkyl, an unsubstituted C₃-C₈ cycloalkyl, an unsubstituted C₅-C₁₂ aryl, or a C₅-C₁₂ aryl substituted with a halogen, a hydroxyl, a haloalkyl, or —OR₁ 2, R₁₂ can be an unsubstituted linear, branched, or cyclic C₁-C₆ alkyl, or a combination thereof.

In some forms, the synthetic compound can have the structure of Formula III:

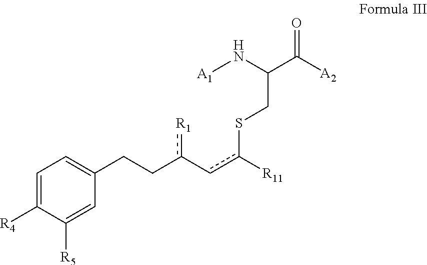

wherein: (i) [symbol] can be a single or double bond; (ii) A₁ and A₂ can be independently an amino acid residue; (iii) R₁ can be an oxygen, a hydroxyl, or —OR₆, and R₆ can be an unsubstituted C₁-C₆ alkyl; (iv) R₄ can be a hydrogen or hydroxyl; (v) R₅ can be a hydrogen, a hydroxyl, or —OR₇, and R₇ can be an unsubstituted C₁-C₆ alkyl; and (vi) R₁₁ can be an unsubstituted branched C₃-C₆ alkyl, an unsubstituted C₃-C₆ cycloalkyl, an unsubstituted phenyl, or a phenyl substituted with a halogen.

In some forms, the synthetic compound can have the structure of Formula IV:

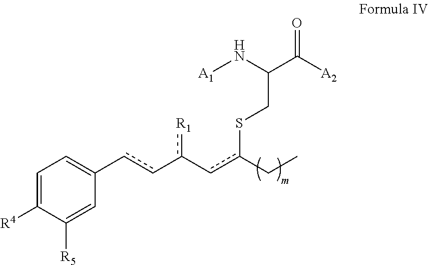

wherein: (i) [symbol] can be a single or double bond; (ii) m can be an integer from 4 to 8; (iii) A₁ and A₂ can be independently an amino acid residue; (iv) R₁ can be an oxygen, a hydroxyl, or —OR₆, and R₆ can be an unsubstituted linear, branched, or cyclic C₁-C₆ alkyl; (v) R₄ can be a hydrogen or hydroxyl; and (vi) R₈ can be a hydrogen, a hydroxyl, or —OR₇, and R₇ can be an unsubstituted linear, branched, or cyclic C₁-C₆ alkyl.

In some forms, the compound can have the structures of Formula V:

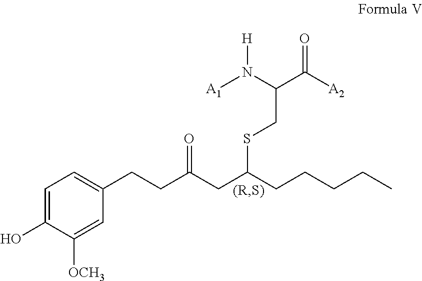

wherein A₁ and A₂ can be independently an amino acid residue.

For Formulae I-V, A₁ can be any suitable amino acid residue (natural or synthetic), as long as the pH of the amino acid forming A₁ is similar to the pH of glutamic acid; and A₂ can be any suitable amino acid residue (natural or synthetic), as long as the pH of the amino acid forming A₂ is similar to the pH of glycine.

The term “similar to” with respect to the pH of an amino acid compared to glutamic acid or glycine means that the pH value of the amino acid is within ±10% of the pH value of glutamic acid or glycine, measured under the same conditions. The term “same conditions” means that the pH measurement was performed using the same concentration of amino acid, the same aqueous medium, the same temperature, the same pressure, etc. It is important that the amino acids forming A₁ and A₂ have similar pH to glutamic acid and glycine respectively to mimic the structure of glutathione (GSH). GSH neutralization is one of the primary prevention mechanisms against reactive oxygen species and electrophiles in living organisms.

The term “residue” with respect to an amino acid means an amino acid molecule minus the hydroxyl group at the C-terminus of the amino acid molecule or one of the hydrogens at the N-terminus of the amino acid molecule. The amino acid forming the amino acid residue of the compounds can be a natural amino acid or a synthetic amino acid. The amino acid forming the amino acid residue of the compounds can be in any isomer form. For example, when the amino acid forming the amino acid residue of the compounds is an aspartic acid, the aspartic acid may be an alpha-aspartic acid or a beta-aspartic acid; when the amino acid molecule is a glutamic acid, the glutamic acid may be an alpha-glutamic acid, a beta-glutamic acid, or a gamma-glutamic acid.

For any of Formulae I-V described above, A₁ can be a natural amino acid residue (such as glutamic acid residue or an aspartic acid residue), or a synthetic amino acid residue where the amino acid forming A₁ has a pH similar to glutamic acid; and A₂ can be a natural amino acid residue (such as an alanine residue, a phenylalanine residue, a leucine residue, a methionine residue, a proline residue, a valine residue, a tryptophan residue, a tyrosine residue, or a glycine residue), or a synthetic amino acid residue where the amino acid forming A₂ has a pH similar to glycine. The natural amino acid forming A₁ of any of Formulae I-V can be in any isomer form, such as alpha-aspartic acid, beta-aspartic acid, alpha-glutamic acid, or gamma-glutamic acid.

In some forms, for any of Formulae I-V described herein, A₁ can be a glutamic acid residue or an aspartic acid residue, or a synthetic derivative thereof; and A₂ can be an alanine residue, a phenylalanine residue, a leucine residue, a methionine residue, a proline residue, a valine residue, a tryptophan residue, a tyrosine residue, or a glycine residue, or a synthetic derivative thereof. In some forms, for any of Formulae I-V described herein, A₁ can be a glutamic acid residue or an aspartic acid residue; and A₂ can be an alanine residue, a phenylalanine residue, a leucine residue, a methionine residue, a proline residue, a valine residue, a tryptophan residue, a tyrosine residue, or a glycine residue.

In some forms, for any of Formulae I-V described herein, A₁ can be a glutamic acid residue or an aspartic acid residue, or a synthetic derivative thereof; and A₂ can be a leucine residue, a methionine residue, a valine residue, a tryptophan residue, an alanine residue, a phenylalanine residue, or a tyrosine residue, or a synthetic derivative thereof.

In some forms, for any of Formulae I-V described herein, A₁ can be a glutamic acid residue or an aspartic acid residue; and A₂ can be a leucine residue, a methionine residue, a valine residue, a tryptophan residue, an alanine residue, a phenylalanine residue, or a tyrosine residue.

In some forms, for any of Formulae I-V described herein, A₁ can be a glutamic acid residue or a synthetic derivative thereof; and A₂ can be a leucine residue, a methionine residue, a valine residue, or a tryptophan residue, or a synthetic derivative thereof. In some forms, for any of Formulae I-V described herein, A₉ can be a glutamic acid residue; and A₂ can be a leucine residue, a methionine residue, a valine residue, or a tryptophan residue.

In some forms, for any of Formulae I-V described herein, A₁ can be an aspartic acid residue or a synthetic derivative thereof; and A₂ can be an alanine residue, a phenylalanine residue, a valine residue, or a tyrosine residue, or a synthetic derivative thereof. In some forms, for any of Formulae I-V described herein, A₁ can be an aspartic acid residue; and A₂ can be an alanine residue, a phenylalanine residue, a valine residue, or a tyrosine residue.

In some forms, for any of Formulae I-V described herein, A₁ can be an alpha-glutamic acid residue, a gamma-glutamic acid residue, an alpha-aspartic acid residue, or a beta-aspartic acid residue; and A₂ can be an alanine residue, a phenylalanine residue, a leucine residue, a methionine residue, a proline residue, a valine residue, a tryptophan residue, a tyrosine residue, or a glycine residue, or a synthetic derivative thereof. In some forms, for any of Formulae I-V described herein, A₁ can be an alpha-glutamic acid residue, a gamma-glutamic acid residue, an alpha-aspartic acid residue, or a beta-aspartic acid residue; and A₂ can be an alanine residue, a phenylalanine residue, a leucine residue, a methionine residue, a proline residue, a valine residue, a tryptophan residue, a tyrosine residue, or a glycine residue.

In some forms, for any of Formulae I-V described herein, A₁ can be an alpha-glutamic acid residue, a gamma-glutamic acid residue, an alpha-aspartic acid residue, or a beta-aspartic acid residue; and A₂ can be a leucine residue, a methionine residue, a valine residue, a tryptophan residue, an alanine residue, a phenylalanine residue, or a tyrosine residue, or s synthetic derivative thereof. In some forms, for any of Formulae I-V described herein, A₁ can be an alpha-glutamic acid residue, a gamma-glutamic acid residue, an alpha-aspartic acid residue, or a beta-aspartic acid residue; and A₂ can be a leucine residue, a methionine residue, a valine residue, a tryptophan residue, an alanine residue, a phenylalanine residue, or a tyrosine residue.

In some forms, for any of Formulae I-V described herein, A₁ can be an alpha-glutamic acid residue or a gamma-glutamic acid residue; and A₂ can be a leucine residue, a methionine residue, a valine residue, or a tryptophan residue, or a synthetic derivative thereof. In some forms, for any of Formulae I-V described herein, A₁ can be an alpha-glutamic acid residue or a gamma-glutamic acid residue; and A₂ can be a leucine residue, a methionine residue, a valine residue, or a tryptophan residue.

In some forms, for any of Formulae I-V described herein, A₁ can be an alpha-aspartic acid residue or a beta-aspartic acid residue; and A₂ can be an alanine residue, a phenylalanine residue, a valine residue, or a tyrosine residue, or a synthetic derivative thereof. In some forms, for any of Formulae I-V described herein, A₁ can be an alpha-aspartic acid residue or a beta-aspartic acid residue; and A₂ can be an alanine residue, a phenylalanine residue, a valine residue, or a tyrosine residue.

For any of Formulae I-V described above, when any of the functional groups and/or substituents is an unsubstituted or substituted alkyl, the alkyl can be a linear C₁-C₁₀ alkyl, a branched C₃-C₁₀ alkyl, a cyclic C₃-C₁₀ alkyl (either monocyclic or polycyclic), a linear C₁-C₉ alkyl, a branched C₃-C₉ alkyl, a cyclic C₃-C₉ alkyl (either monocyclic or polycyclic), a linear C₁-C₈ alkyl, a branched C₃-C₈ alkyl, a cyclic C₃-C₅ alkyl (either monocyclic or polycyclic), a linear C₁-C₇ alkyl, a branched C₃-C₇ alkyl, a cyclic C₃-C₇ alkyl (either monocyclic or polycyclic), a linear C₁-C₆ alkyl, a branched C₃-C₆ alkyl, a cyclic C₃-C₆ alkyl, a linear C₁-C₅ alkyl, a cyclic C₃-C₅ alkyl, a branched C₃-C₅ alkyl, a linear C₁-C₄ alkyl, a cyclic C₃-C₄ alkyl, and a branched C₃-C₄ alkyl, such as a linear C₁, C₂, C₃, C₄, C₅, C₆ alkyl, a branched C₃, C₄, C₅, C₆ alkyl, or a cyclic C₃, C₄, C₅, C₆ alkyl, for example, methyl, ethyl, n-propyl, isopropyl, cyclopropyl, n-butyl, sec-butyl, isobutyl, tert-butyl, cyclobutyl, n-pentyl, tert-pentyl, neopentyl, isopentyl, sec-pentyl, 3-pentyl, sec-isopentyl, active pentyl, cyclopentyl, n-hexyl, cyclohexyl, etc. It is understood that any of the exemplary alkyl groups described above can be a heteroalkyl. For example, the alkyl can be a linear C₂-C₆ heteroalkyl, a branched C₃-C₆ heteroalkyl, or a cyclic C₃-C₆ heteroalkyl (i.e., a heterocycloalkyl). the alkyl can be a linear alkyl, a branched alkyl, or a cyclic alkyl (either monocyclic or polycyclic).

For any of Formula I-V described above, when any of the functional groups and/or substituents is an unsubstituted or substituted aryl (monoaryl or polyaryl), the aryl can be a C₅-C₁ aryl, a C₅-C₁₁ aryl, a C₅-C₉ aryl, a C₆-C₁₂ aryl, a C₆-C₁₁ aryl, or a C₆-C₉ aryl. It is understood that the aryl can be a heteroaryl, such as a C₅-C₁₂ heteroaryl, a C₅-C₁₁ heteroaryl, a C₅-C₉ heteroaryl, a C₆-C₁₂ heteroaryl, a C₆-C₁₁ heteroaryl, or a C₆-C₉ heteroaryl.

The synthetic compounds may contain one or more chiral centers or may otherwise be capable of existing as multiple stereoisomers. These may be pure (single) stereoisomers or mixtures of stereoisomers, such as enantiomers, diastereomers, and enantiomerically or diastereomerically enriched mixtures. The synthetic compounds may be capable of existing as geometric isomers. Accordingly, it is to be understood that the present invention includes pure geometric isomers or mixtures of geometric isomers.

### 2. Exemplary Compounds

Exemplary synthetic compounds are presented below.

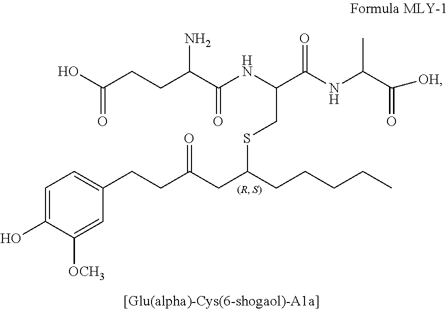 [SMILES: CCCCCC(CC(=O)CCc1ccc(O)c(OC)c1)SCC(NC(=O)C(N)CCC(=O)O)C(=O)NC(C)C(=O)O]

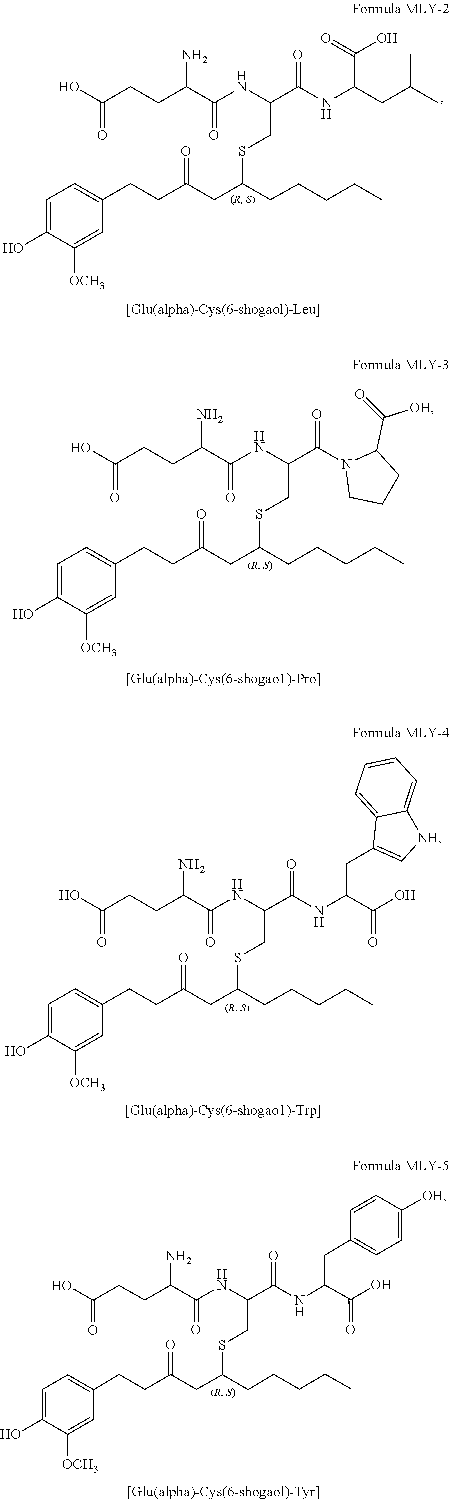 [SMILES: CCCCCC(CC(=O)CCc1ccc(O)c(OC)c1)SCC(NC(=O)C(N)CCC(=O)O)C(=O)NC(CC(C)C)C(=O)O ; CCCCCC(CC(=O)CCc1ccc(O)c(OC)c1)SCC(NC(=O)C(N)CCC(=O)O)C(=O)N1CCCC1C(=O)O ; CCCCCC(CC(=O)CCc1ccc(O)c(OC)c1)SCC(NC(=O)C(N)CCC(=O)O)C(=O)NC(Cc1c[nH]c2ccccc12)C(=O)O ; CCCCCC(CC(=O)CCc1ccc(O)c(OC)c1)SCC(NC(=O)C(N)CCC(=O)O)C(=O)NC(Cc1ccc(O)cc1)C(=O)O]

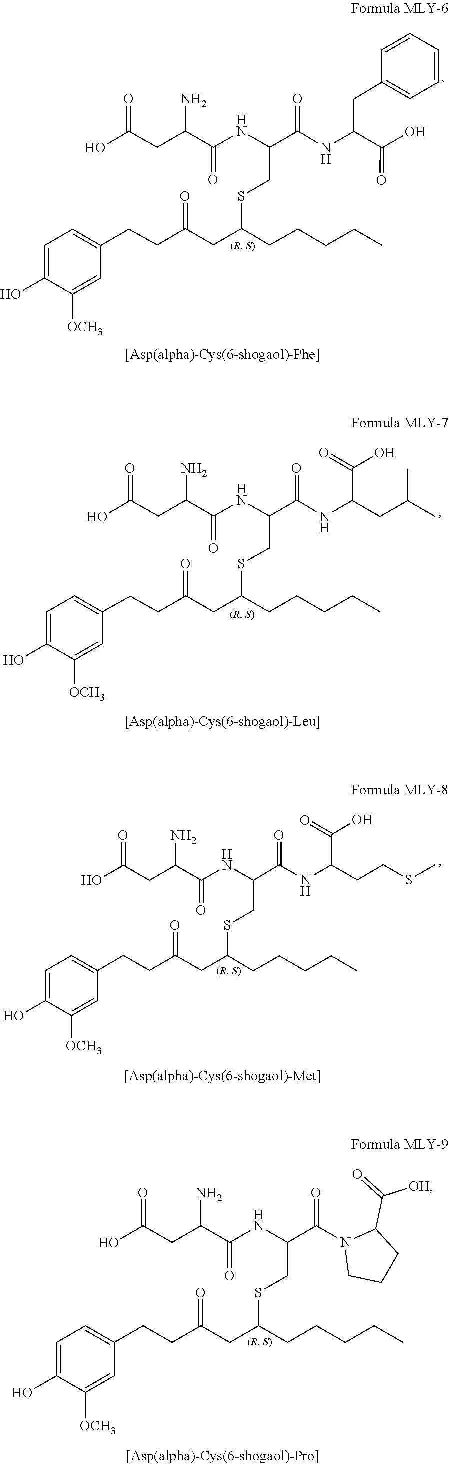 [SMILES: CCCCCC(CC(=O)CCc1ccc(O)c(OC)c1)SCC(NC(=O)C(N)CC(=O)O)C(=O)NC(Cc1ccccc1)C(=O)O ; CCCCCC(CC(=O)CCc1ccc(O)c(OC)c1)SCC(NC(=O)C(N)CC(=O)O)C(=O)NC(CC(C)C)C(=O)O ; CCCCCC(CC(=O)CCc1ccc(O)c(OC)c1)SCC(NC(=O)C(N)CC(=O)O)C(=O)NC(CCSC)C(=O)O ; CCCCCC(CC(=O)CCc1ccc(O)c(OC)c1)SCC(NC(=O)C(N)CC(=O)O)C(=O)N1CCCC1C(=O)O]

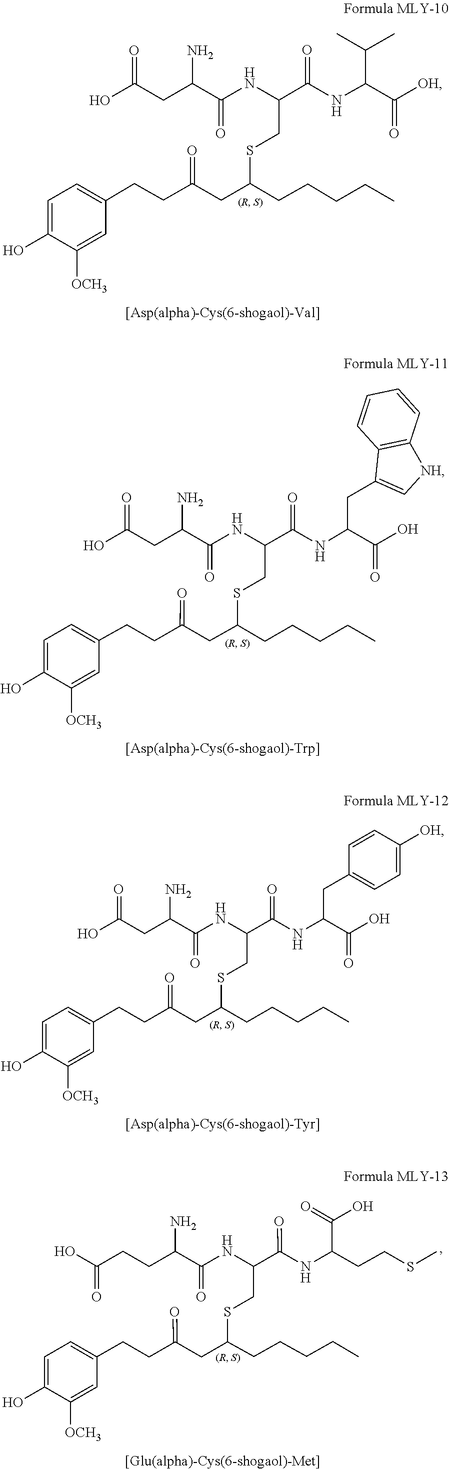 [SMILES: CCCCCC(CC(=O)CCc1ccc(O)c(OC)c1)SCC(NC(=O)C(N)CC(=O)O)C(=O)NC(C(=O)O)C(C)C ; CCCCCC(CC(=O)CCc1ccc(O)c(OC)c1)SCC(NC(=O)C(N)CC(=O)O)C(=O)NC(Cc1c[nH]c2ccccc12)C(=O)O ; CCCCCC(CC(=O)CCc1ccc(O)c(OC)c1)SCC(NC(=O)C(N)CC(=O)O)C(=O)NC(Cc1ccc(O)cc1)C(=O)O ; CCCCCC(CC(=O)CCc1ccc(O)c(OC)c1)SCC(NC(=O)C(N)CCC(=O)O)C(=O)NC(CCSC)C(=O)O]

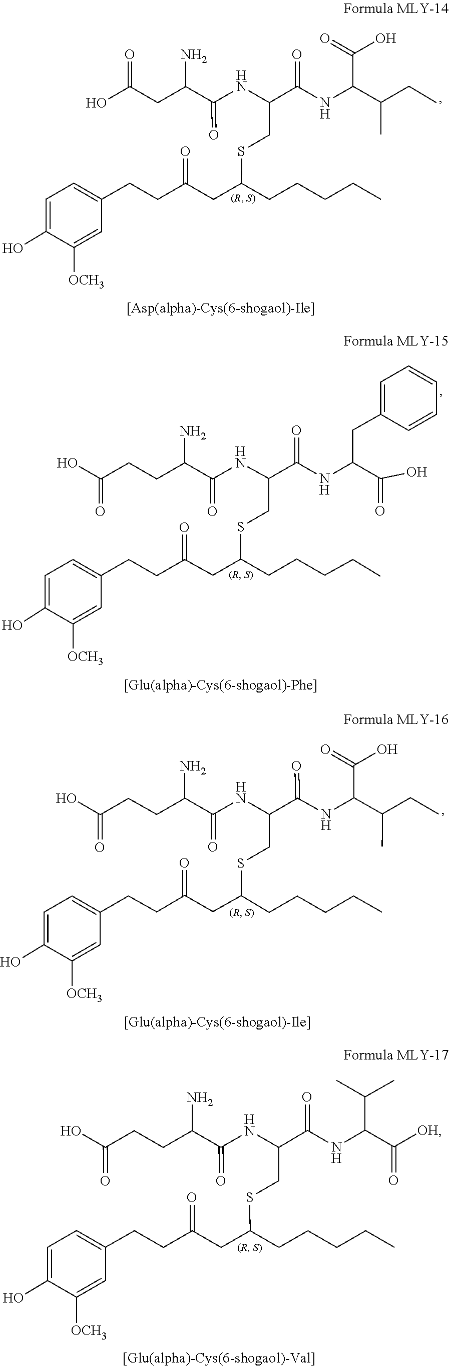 [SMILES: CCCCCC(CC(=O)CCc1ccc(O)c(OC)c1)SCC(NC(=O)C(N)CC(=O)O)C(=O)NC(C(=O)O)C(C)CC ; CCCCCC(CC(=O)CCc1ccc(O)c(OC)c1)SCC(NC(=O)C(N)CCC(=O)O)C(=O)NC(Cc1ccccc1)C(=O)O ; CCCCCC(CC(=O)CCc1ccc(O)c(OC)c1)SCC(NC(=O)C(N)CCC(=O)O)C(=O)NC(C(=O)O)C(C)CC ; CCCCCC(CC(=O)CCc1ccc(O)c(OC)c1)SCC(NC(=O)C(N)CCC(=O)O)C(=O)NC(C(=O)O)C(C)C]

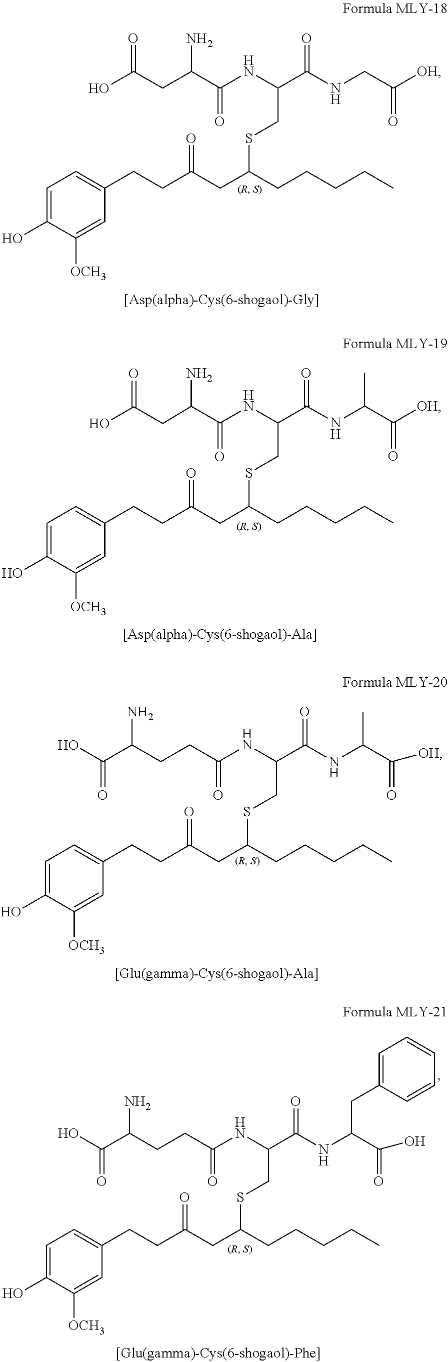 [SMILES: CCCCCC(CC(=O)CCc1ccc(O)c(OC)c1)SCC(NC(=O)C(N)CC(=O)O)C(=O)NCC(=O)O ; CCCCCC(CC(=O)CCc1ccc(O)c(OC)c1)SCC(NC(=O)C(N)CC(=O)O)C(=O)NC(C)C(=O)O ; CCCCCC(CC(=O)CCc1ccc(O)c(OC)c1)SCC(NC(=O)CCC(N)C(=O)O)C(=O)NC(C)C(=O)O ; CCCCCC(CC(=O)CCc1ccc(O)c(OC)c1)SCC(NC(=O)CCC(N)C(=O)O)C(=O)NC(Cc1ccccc1)C(=O)O]

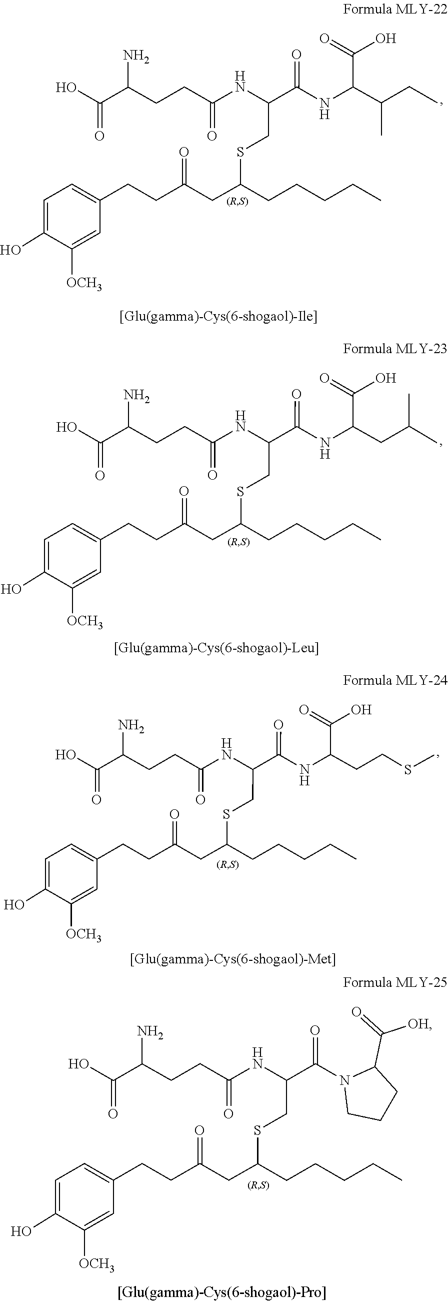 [SMILES: CCCCCC(CC(=O)CCc1ccc(O)c(OC)c1)SCC(NC(=O)CCC(N)C(=O)O)C(=O)NC(C(=O)O)C(C)CC ; CCCCCC(CC(=O)CCc1ccc(O)c(OC)c1)SCC(NC(=O)CCC(N)C(=O)O)C(=O)NC(CC(C)C)C(=O)O ; CCCCCC(CC(=O)CCc1ccc(O)c(OC)c1)SCC(NC(=O)CCC(N)C(=O)O)C(=O)NC(CCSC)C(=O)O ; CCCCCC(CC(=O)CCc1ccc(O)c(OC)c1)SCC(NC(=O)CCC(N)C(=O)O)C(=O)N1CCCC1C(=O)O]

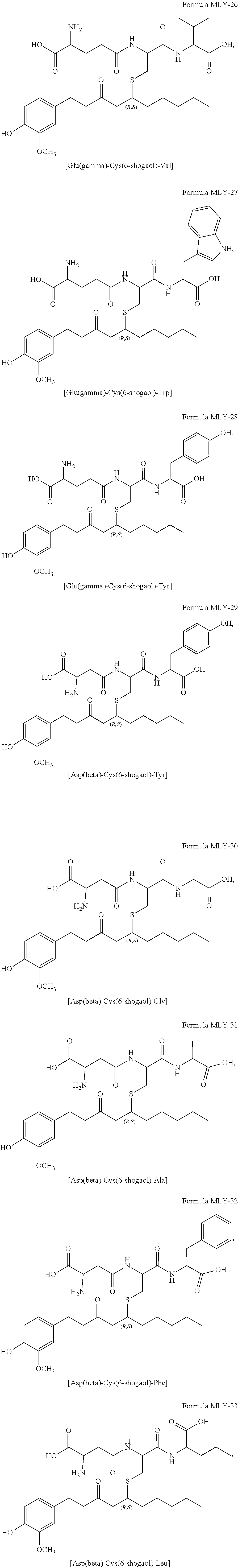

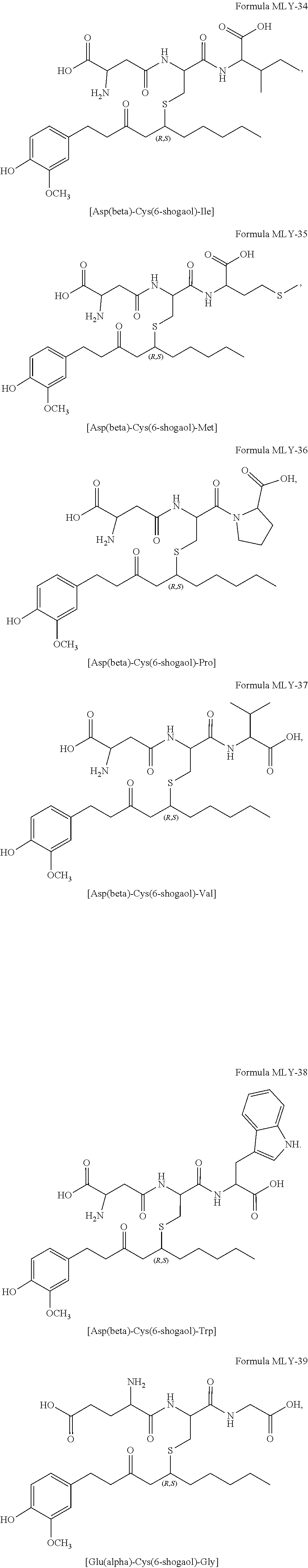 [SMILES: CCCCCC(CC(=O)CCc1ccc(O)c(OC)c1)SCC(NC(=O)CC(N)C(=O)O)C(=O)NC(C(=O)O)C(C)CC ; CCCCCC(CC(=O)CCc1ccc(O)c(OC)c1)SCC(NC(=O)CC(N)C(=O)O)C(=O)NC(CCSC)C(=O)O ; CCCCCC(CC(=O)CCc1ccc(O)c(OC)c1)SCC(NC(=O)CC(N)C(=O)O)C(=O)N1CCCC1C(=O)O ; CCCCCC(CC(=O)CCc1ccc(O)c(OC)c1)SCC(NC(=O)CC(N)C(=O)O)C(=O)NC(C(=O)O)C(C)C ; CCCCCC(CC(=O)CCc1ccc(O)c(OC)c1)SCC(NC(=O)CC(N)C(=O)O)C(=O)NC(Cc1c[nH]c2ccccc12)C(=O)O ; CCCCCC(CC(=O)CCc1ccc(O)c(OC)c1)SCC(NC(=O)C(N)CCC(=O)O)C(=O)NCC(=O)O]

### 3. Pharmaceutically Acceptable Salts

The synthetic compounds disclosed herein may be neutral or may be one or more pharmaceutically acceptable salts, crystalline forms, non-crystalline forms, hydrates, or solvates, or a combination thereof. References to the synthetic compounds may refer to the neutral molecule, and/or those additional forms thereof collectively and individually from the context. Pharmaceutically acceptable salts of the synthetic compounds include the acid addition and base salts thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

### B. Pharmaceutical Formulations

Pharmaceutical formulations that contain M13, or one or more of the synthetic compounds disclosed herein, or a combination thereof, in a form suitable for administration to a mammal, are disclosed. Typically, M13, or the disclosed synthetic compound(s), or a combination thereof, in the pharmaceutical formulation is present in an amount effective to treat a viral infection in a subject. In some forms, the synthetic compound(s) in the pharmaceutical formulation is present in an amount effective to treat an inflammation and/or a viral infection in a subject. In some forms, the viral infection is a respiratory viral infection, such as SARS-CoV-2 infection.

The pharmaceutical formulation containing M13, and/or the disclosed synthetic compound(s) may also include one more pharmaceutically acceptable carrier and/or one or more pharmaceutically acceptable excipients. For example, the pharmaceutical formulation may be in the form of a liquid, such as a solution or a suspension, and contain one or more of M13, and the disclosed synthetic compounds in an aqueous medium and, optionally, one or more suitable excipients for the liquid formulation. For example, the pharmaceutical formulation may be in a solid form, such as a tablet or powders, and contain one or more of M13, and the disclosed synthetic compounds and one or more suitable excipients for the solid formulation.

Optionally, the pharmaceutical formulation is in a liquid form, and contains one or more of the disclosed compounds in an aqueous medium and, optionally, one or more suitable excipients for the liquid formulation. Optionally, the pharmaceutical formulation is in a solid form, and contains one or more of the disclosed synthetic compounds and one or more suitable excipients for a solid formulation. Optionally, the pharmaceutical formulation may be in the form of a liquid, such as a solution or a suspension, and contain M13 in an aqueous medium and, optionally, one or more suitable excipients for the liquid formulation. Optionally, the pharmaceutical formulation is in a solid form, and contains M13 and one or more suitable excipients for a solid formulation.

The pharmaceutical formulation may include a second active agent, optionally more than one second active agent. The second active agent can be an anti-inflammatory agent or an antiviral agent that is different from M13 and the synthetic compounds disclosed herein.

### 1. Carriers and Excipients

The pharmaceutical formulation can contain one or more pharmaceutically acceptable carriers and/or one or more pharmaceutically acceptable excipients. Suitable pharmaceutically acceptable carriers and excipients are generally recognized as safe (GRAS), and may be administered to an individual without causing undesirable biological side effects or unwanted interactions.

Representative carriers and excipients that can be used in the pharmaceutical formulations include solvents (including buffers), diluents, pH modifying agents, preservatives, antioxidants, suspending agents, wetting agents, viscosity modifiers, tonicity agents, and stabilizing agents, and a combination thereof.

In some forms, the compounds can be dissolved or suspended in a suitable carrier to form a liquid pharmaceutical formulation, such as sterile saline, phosphate buffered saline (PBS), balanced salt solution (BSS), viscous gel, or other pharmaceutically acceptable carriers for administration. The pharmaceutical formulation may also be a sterile solution, suspension, or emulsion in a nontoxic, parenterally acceptable diluent or solvent.

Excipients can be added to a liquid or solid pharmaceutical formulation to assist in sterility, stability (e.g., shelf-life), integration, and to adjust and/or maintain pH or isotonicity of the compounds in the pharmaceutical formulation, such as diluents, pH modifying agents, preservatives, antioxidants, suspending agents, wetting agents, viscosity modifiers, tonicity agents, and stabilizing agents, and a combination thereof.

### 2. Form

The pharmaceutical formulation containing M13, or one or more of the disclosed synthetic compounds, or a combination thereof, can be in a liquid form or a solid form, as a liquid formulation or a solid formulation for oral administration or parenteral administration (e.g., intramuscular administration, intravenous administration, intraperitoneal administration, and subcutaneous administration) to a subject.

a. Oral Formulations

In some forms, the pharmaceutical formulation containing M13, or one or more of the disclosed compounds, or a combination thereof, can be in a form suitable for oral administration to a subject, such as a mammal (i.e., an oral formulation). Oral administration may involve swallowing, so that the synthetic compound(s) enter the gastrointestinal tract, or buccal or sublingual administration may be employed by which the compound(s) enter(s) the blood stream directly from the mouth.

Formulations suitable for oral administration include solid formulations such as tablets, capsules containing particulates, liquids, powders, lozenges (including liquid-filled lozenges), chews, multi- and nano-particulates, gels, solid solutions, liposomes, films, ovules, sprays, and liquid formulations.

Liquid formulations for oral administration include suspensions, solutions, syrups, and elixirs. Such oral formulations may be employed as fillers in soft or hard capsules and can contain one or more suitable carriers and/or excipients, for example, water, ethanol, polyethylene glycol, propylene glycol, chitosan polymers and chitosan derivatives (e.g., N-trimethylene chloride chitosan, chitosan esters, chitosan modified with hydrophilic groups, such as amino groups, carboxyl groups, sulfate groups, etc.), methylcellulose, a suitable oil, one or more emulsifying agents, and/or suspending agents. Liquid formulations for oral administration may also be prepared by the reconstitution of a solid, for example, from a sachet.

Optionally, M13, or one or more of the disclosed compounds, or a combination thereof, is/are included in a fast-dissolving and/or fast-disintegrating dosage form.

For tablet or capsule dosage forms, in addition to M13, and/or one or more of the disclosed compounds described herein, tablets generally contain disintegrants, binders, diluents, surface active agents, lubricants, glidants, antioxidants, colorants, flavoring agents, preservatives, or taste masking agents, or a combination thereof.

Examples of suitable disintegrants for forming a tablet or capsule dosage form include, but are not limited to, sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant can have a concentration in a range from about 1 wt % to about 25 wt %, from about 5 wt % to about 20 wt % of the tablet or capsule dosage form containing M13, or one or more the disclosed compounds, or a combination thereof.

Binders are generally used to impart cohesive qualities to a tablet formulation. Suitable binders for forming a tablet or capsule formulation containing M13, or one or more the disclosed synthetic compounds, or a combination thereof, include, but are not limited to, microcrystalline cellulose, gelatin, sugars, polyethylene glycol, natural and synthetic gums, polyvinylpyrrolidone, pregelatinized starch, chitosan polymers and chitosan derivatives (e.g. N-trimethylene chloride chitosan, chitosan esters, chitosan modified with hydrophilic groups, such as amino groups, carboxyl groups, sulfate groups, etc.), hydroxypropyl cellulose, and hydroxypropyl methylcellulose.

Suitable diluents for forming a tablet or capsule formulation include, but are not limited to, lactose (as, for example, the monohydrate, spray-dried monohydrate or anhydrous form), chitosan polymers and chitosan derivatives (e.g. N-trimethylene chloride chitosan, chitosan esters, chitosan modified with hydrophilic groups, such as amino groups, carboxyl groups, sulfate groups, etc.), N-sulfonated derivatives of chitosan, quaternarized derivatives of chitosan, carbosyalkylated chitosan, microcrystalline chitosan, mannitol, xylitol, dextrose, sucrose, sorbitol, microcrystalline cellulose, starch and dibasic calcium phosphate dihydrate.

Tablet or capsule formulations containing M13, or one or more the disclosed synthetic compounds, or a combination thereof, may also contain surface active agents, such as sodium lauryl sulfate and polysorbate 80, and glidants such as silicon dioxide and talc. When present, surface active agents can have a concentration in a range from about 0.2 wt % to 5 wt % of the tablet or capsule formulation.

Tablet or capsule formulations containing M13, or one or more the disclosed synthetic compounds, or a combination thereof, also can contain lubricants, such as magnesium stearate, calcium stearate, zinc stearate, sodium stearyl fumarate, and mixtures of magnesium stearate with sodium lauryl sulphate. Lubricants can have a concentration in a range from about 0.25 wt % to 10 wt %, from about 0.5 wt % to about 3 wt % of the tablet or capsule formulation.

Other possible excipients included in a tablet or capsule formulation containing M13, or one or more the disclosed synthetic compounds, or a combination thereof, include glidants (e.g., Talc or colloidal anhydrous silica at about 0.1 wt % to about 3 wt % of the tablet or capsule formulation), antioxidants, colorants, flavoring agents, preservatives, and taste-masking agents. When present, glidants can have a concentration in a range from about 0.2 wt % to 1 wt % of the tablet or capsule formulation.

An exemplary tablet formulation contains up to about 80 wt % of the compound(s) described herein, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet or capsule blends, including M13, or one or more the disclosed synthetic compounds, or a combination thereof, and one or more suitable excipients, may be compressed directly or by roller to form tablets. Tablet or capsule blends or portions of the blends may alternatively be wet-, dry-, or melt-granulated, melt congealed, or extruded before tableting. The final tablet or capsule formulation may contain one or more layers and may be coated or uncoated; it may even be encapsulated in a particle, such as a polymeric particle or a liposomal particle.

Solid formulations containing M13, or one or more the disclosed synthetic compounds, or a combination thereof, for oral administration may be formulated to be immediate and/or modified release. Modified release formulations include delayed, sustained, pulsed, controlled, targeted, and programmed release formulations.

b. Parenteral Formulations

In some forms, the pharmaceutical formulation containing M13, or one or more the disclosed synthetic compounds, or a combination thereof, can be in a form suitable for administration directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intramuscular, and subcutaneous delivery. Suitable means for parenteral administration include needle (including microneedle) injectors, needle-free injectors, and infusion techniques.

For example, the pharmaceutical formulation containing M13, or one or more the disclosed synthetic compounds, or a combination thereof, are in a form suitable for intramuscular administration, intravenous administration, intraperitoneal administration, or subcutaneous administration, or a combination thereof.

Parenteral formulations containing M13, or one or more the disclosed synthetic compounds, or a combination thereof, described herein are typically aqueous solutions which can contain excipients such as salts, carbohydrates and buffering agents (e.g., from about pH 6.5 to about pH 8.0, from about pH 6.5 to about pH 7.4, from about pH 6.5 to about pH 7.0, from about pH 7.0 to pH 8.0, or from about pH 7.0 to about pH 7.4), but, for some applications, they may be more suitably formulated as a sterile aqueous solution or as a dried form to be used in conjunction with a suitable vehicle such as sterile, pyrogen-free water.

The liquid formulations containing M13, or one or more the disclosed synthetic compounds, or a combination thereof, for parenteral administration may be a solution, a suspension, or an emulsion.

The liquid pharmaceutically acceptable carrier forming the parenteral formulation containing M13, or one or more the disclosed synthetic compounds, or a combination thereof, can include one or more physiologically compatible buffers, such as a phosphate buffer. One skilled in the art can readily determine a suitable saline content and pH for an aqueous carrier for administration (e.g., from about pH 6.5 to about pH 8.0, from about pH 6.5 to about pH 7.4, from about pH 6.5 to about pH 7.0, from about pH 7.0 to pH 8.0, or from about pH 7.0 to about pH 7.4).

Liquid formulations containing M13, or one or more the disclosed synthetic compounds, or a combination thereof, for parenteral administration may include one or more suspending agents, such as cellulose derivatives, sodium alginate, polyvinylpyrrolidone, gum tragacanth, or lecithin. The liquid formulations may also include one or more preservatives, such as ethyl or n-propyl p-hydroxybenzoate.

In some forms, the liquid formulation containing M13, or one or more the disclosed synthetic compounds, or a combination thereof, contains one or more solvents that are low toxicity organic (i.e., nonaqueous) class 3 residual solvents, such as ethanol, acetone, ethyl acetate, tetrahydrofuran, ethyl ether, and propanol, and a combination thereof. Any such solvents included in the liquid formulation should not detrimentally react with any of M13, and the disclosed synthetic compounds, and any additional active agents when present in the liquid formulation. Solvents such as freon, alcohol, glycol, polyglycol, or fatty acid, can also be included in the liquid formulation containing M13, or one or more the disclosed synthetic compounds, or a combination thereof, as desired to increase the volatility of the solution or suspension.

Liquid formulations containing M13, or one or more the disclosed synthetic compounds, or a combination thereof, for parenteral administration may also contain minor amounts of polymers, surfactants, or other pharmaceutically acceptable excipients known to those in the art. In this context, “minor amounts” means an amount that is sufficiently small to avoid adversely affecting uptake of any of M13, and the disclosed synthetic compound(s), by the targeted cells, such as pituitary gonadotrophs.

The preparation of parenteral formulations containing M13, or one or more the disclosed synthetic compounds, or a combination thereof, is typically under sterile conditions, for example, by lyophilization, which can be accomplished using standard pharmaceutical techniques known to those skilled in the art.

Formulations for parenteral administration containing M13, M13-2, or one or more the disclosed compounds, or a combination thereof, may be formulated to provide immediate and/or modified release of the active agent. Modified release formulations include delayed, sustained, pulsed, controlled, targeted, and programmed release formulations.

c. Pulmonary and Mucosal Formulations

In some forms, the pharmaceutical formulation containing M13, or one or more of the disclosed synthetic compounds, or a combination thereof, can be in a form suitable for pulmonary or mucosal administration. The administration can include delivery of the composition to the lungs, nasal, oral (sublingual, buccal), vaginal, or rectal mucosa.

For example, M13, or one or more the disclosed synthetic compounds, or a combination thereof, can be administered intranasally or by oral inhalation, such as in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (such as an atomizer using electro hydrodynamics to produce a fine mist), or nebulizer, with or without the use of a suitable propellant, such as water, ethanol-water mixture, 1,1,1,2-tetrafluoroethane or 1,1,1,2,3,3,3-heptafluoropropane. For intranasal or oral inhalation use, the powder may comprise a bioadhesive agent, for example, chitosan or cyclodextrin. The term aerosol as used herein refers to any preparation of a fine mist of particles, which can be in solution or a suspension, whether or not it is produced using a propellant. Aerosols can be produced using standard techniques, such as ultrasonication or high-pressure treatment.

The pressurized container, pump, spray, atomizer, or nebulizer contains a solution or suspension of M13, or the disclosed synthetic compound(s), or a combination thereof, including, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid.

Prior to use in a dry powder or suspension formulation, a drug product is micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying.

Capsules (made, for example, from gelatin or hydroxypropyl methylcellulose), blisters and cartridges for use in an inhaler or insufflator may be formulated to contain a powder mix of M13, or one or more of the disclosed synthetic compounds, or a combination thereof; a suitable powder base such as lactose or starch; and a performance modifier such as 1-leucine, mannitol, or magnesium stearate. The lactose may be anhydrous or in the form of the monohydrate, preferably the latter. Other suitable excipients include dextran, glucose, maltose, sorbitol, xylitol, fructose, sucrose, and trehalose.

A suitable solution formulation containing M13, or one or more the disclosed synthetic compounds, or a combination thereof, for use in an atomizer using electro hydrodynamics to produce a fine mist may contain from 1 μg to 20 mg of M13, or one or more the disclosed synthetic compounds, or a combination thereof, per actuation and the actuation volume may vary from 1 μl to 100 μl. A typical formulation may contain M13, or one or more the disclosed synthetic compounds, or a combination thereof; propylene glycol; sterile water; ethanol; and sodium chloride. Alternative solvents that may be used instead of propylene glycol include glycerol and polyethylene glycol.

Suitable flavors, such as menthol and levomenthol, or sweeteners, such as saccharin or saccharin sodium, may be added to those formulations intended for inhaled/intranasal administration.

Formulations for inhaled/intranasal administration may be formulated to be immediate and/or modified release using, for example, PGLA. Modified release formulations include delayed, sustained, pulsed, controlled, targeted, and programmed release formulations.

In the case of dry powder inhalers and aerosols, the dosage unit is determined by means of a valve which delivers a metered amount. Units in accordance with the compounds are typically arranged to administer a metered dose or “puff.” The overall daily dose will be administered in a single dose or, more usually, as divided doses throughout the day.

In some forms, M13, or one or more the disclosed synthetic compounds, or a combination thereof, can be formulated for pulmonary delivery, such as intranasal administration or oral inhalation. Carriers for pulmonary formulations can be divided into those for dry powder formulations and for administration as solutions. For administration via the upper respiratory tract, the formulation can be formulated into an aqueous solution, e.g., water or isotonic saline, buffered or un-buffered, or as an aqueous suspension, for intranasal administration as drops or as a spray. Such aqueous solutions or suspensions may be isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. One skilled in the art can readily determine a suitable saline content and pH for an innocuous aqueous solution for nasal and/or upper respiratory administration.

In some forms, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In some forms, solvents that are low toxicity organic (i.e., nonaqueous) class 3 residual solvents, such as ethanol, acetone, ethyl acetate, tetrahydrofuran, ethyl ether, and propanol may be used for the formulations containing M13, or one or more the disclosed synthetic compounds, or a combination thereof. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In some forms, the pharmaceutical formulations containing M13, or one or more the disclosed synthetic compounds, or a combination thereof, may contain minor amounts of polymers, surfactants, or other excipients well known to those of the art. In this context, “minor amounts” means no excipients are present that might affect or mediate uptake of the compounds by cells and that the excipients that are present in amount that do not adversely affect uptake of M13, and the disclosed synthetic compound(s), by cells.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Non-aqueous suspensions of lipids can also be prepared in absolute ethanol using a reusable PARI LC Jet+ nebulizer (PARI Respiratory Equipment, Monterey, CA).

d. Topical Formulations M13, or one or more the disclosed synthetic compounds, or a combination thereof, can be administered directly to the external surface of the skin or the mucous membranes (including the surface membranes of the nose, lungs and mouth), such that the M13, or disclosed synthetic compound(s), or a combination thereof, can cross the external surface of the skin or mucous membrane and enters the underlying tissues.

Formulations for topical administration generally contain a dermatologically acceptable carrier that is suitable for application to the skin, has good aesthetic properties, is compatible with the active agents and any other components, and will not cause any untoward safety or toxicity concerns.

The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. These emulsions can also be delivered in the form of sprays using either mechanical pump containers or pressurized aerosol containers using conventional propellants. These carriers can also be delivered in the form of a mousse or a transdermal patch. Other suitable topical carriers include anhydrous liquid solvents such as oils, alcohols, and silicones (e.g., mineral oil, ethanol isopropanol, dimethicone, cyclomethicone, and the like); aqueous-based single phase liquid solvents (e.g., hydro-alcoholic solvent systems, such as a mixture of ethanol and/or isopropanol and water); and thickened versions of these anhydrous and aqueous-based single phase solvents (e.g. where the viscosity of the solvent has been increased to form a solid or semi-solid by the addition of appropriate gums, resins, waxes, polymers, salts, and the like). Examples of topical carrier systems useful in the present formulations are described in the following four references all of which are incorporated herein by reference in their entirety: “Sun Products Formulary” Cosmetics & Toiletries, vol. 105, pp. 122-139 (December 1990); “Sun Products Formulary,” Cosmetics & Toiletries, vol. 102, pp. 117-136 (March 1987); U.S. Pat. No. 5,605,894 to Blank et al., and U.S. Pat. No. 5,681,852 to Bissett.

Formulations containing M13, or one or more the disclosed synthetic compounds, or a combination thereof, for topical administration may be formulated to be immediate and/or modified release. Modified release formulations include delayed, sustained, pulsed, controlled, targeted, and programmed release formulations. Thus, the compounds may be formulated as a solid, semi-solid, or thixotropic liquid for administration as an implanted depot providing modified release of the synthetic compounds. Examples of such formulations include drug-coated stents and poly(dl-lactic-coglycolic)acid (PGLA) microspheres.

### 3. Additional Active Agent(s)

The pharmaceutical formulation disclosed herein may contain, or be otherwise co-administered with, one or more additional active agents (in addition to M13, and the disclosed synthetic compounds), such as an anti-inflammatory agent or an antiviral agent, or a combination thereof.

The total amount of the additional active agents in the pharmaceutical formulation can be in a range from about 0.01 wt % to about 10 wt %; from about 0.01 wt % to about 1 wt %; from about 0.01 wt % to about 0.75 wt %; or from about 0.1 wt % to about 0.5 wt % of the pharmaceutical formulation. The term “total amount of the additional active agents” refers to the total weight of the additional active agents, such as anti-inflammatory agents and/or antiviral agents, in the pharmaceutical formulation relative to the weight of the pharmaceutical formulation.

a. Anti-inflammatory Agents

Optionally, the disclosed pharmaceutical formulation contains one or more additional anti-inflammatory agents. Suitable anti-inflammatory agents for use in the disclosed pharmaceutical formulation include, but are not limited to, steroids, such as clobetasol, halobetasol, halcinonide, amcinonide, betamethasone, desoximetasone, diflucortolone, fluocinolone, fluocinonide, mometasone, clobetasone, desonide, hydrocortisone, prednicarbate, and triamcinolone, salts thereof, and combinations thereof; non-steroidal anti-inflammatory drugs, such as aceclofenac, aspirin, celecoxib, clonixin, dexibupifen, dexketoprofen, diclofenac, diflunisal, droxicam, etodolac, etoricoxib, fenoprofen, flufenamic acid, flurbiprofen, ibuprofen, indomethacin, isoxicam, ketoprofen, ketorolac, licofelone, lornoxicam, loxoprofen, lumiracoxib, meclofenamic acid, mefenamic acid, meloxicam, nabumetone, naproxen, nimesulide, oxaprozin, parecoxib, phenylbutazone, piroxicam, rofecoxib, salsalate, sulindac, tenoxicam, tolfenamic acid, tolmetin, and valdecoxib, and combinations thereof; and combinations thereof.

b. Antiviral Agents

Optionally, the disclosed pharmaceutical formulation contains one or more additional antiviral agents, such as those that can kill or inactivate respiratory virus such as a coronavirus, for example, SARS-CoV-2. Suitable antiviral agents for use in the disclosed pharmaceutical formulation include, but are not limited to, chloroquine, darunavir, galidesivir, interferon beta, lopinavir, ritonavir, remdesivir, and triazavirin, and combinations thereof.

### 4. Dosages/wt % Concentration

In some forms, the pharmaceutical formulation contains an effective amount of M13 and/or one or more of the disclosed synthetic compounds for any one or several of the disclosed treatments. For example, the effective amount can be an amount effective to treat a microbial infection, an inflammatory disease or disorder, a cancer, one or more symptoms and/or conditions associated with a microbial infection, one or more symptoms and/or conditions associated with an inflammatory disease or disorder, and/or one or more symptoms and/or conditions associated with a cancer. An effective amount or therapeutically effective amount means a dosage and/or other element (e.g., amount of time) sufficient to treat, inhibit, or alleviate one or more symptoms of a disease state being treated or to otherwise provide a desired pharmacologic and/or physiologic effect.

For example, when the pharmaceutical formulation contains M13, or one or more the disclosed synthetic compound(s), the amount of the M13, or the total amount of the synthetic compound(s) collectively in the pharmaceutical formulation is effective to treat a viral infection, such as a respiratory viral infection, for example, SARS-CoV-2 infection. The term “total amount of the compound(s) collectively” refers to the total weight of the compound(s) in the pharmaceutical formulation relative to the weight of the pharmaceutical formulation.

When the pharmaceutical formulation contains two or more of M13, and the disclosed synthetic compound(s), the total amount of the M13, and/or the disclosed synthetic compound(s) collectively in the pharmaceutical formulation is effective to treat a viral infection, such as a respiratory viral infection, for example, SARS-CoV-2 infection. The term “total amount of the M13, and/or the disclosed synthetic compound(s) collectively” refers to the total weight of M13, /or synthetic compound(s) in the pharmaceutical formulation relative to the weight of the pharmaceutical formulation. For example, when the pharmaceutical formulation contains a combination of M13 and one or more the disclosed synthetic compounds, the total amount refers to the total weight of M13 and the disclosed synthetic compounds in the pharmaceutical formulation relative to the weight of the pharmaceutical formulation. For example, when the pharmaceutical formulation contains a combination of M13, and one or more the disclosed synthetic compounds, the total amount refers to the total weight of M13, and the disclosed synthetic compounds in the pharmaceutical formulation relative to the weight of the pharmaceutical formulation.

When the pharmaceutical formulation contains one or more the disclosed synthetic compound(s), the total amount of the compound(s) collectively is effective to treat a viral infection (such as a respiratory viral infection, for example, SARS-CoV-2 infection) and/or an inflammatory disease or disorder.

The precise dosage will vary according to a variety of factors such as subject-dependent variables (e.g., age, immune system health, etc.), the disease, and the treatment being administered.

For example, when the pharmaceutical formulation contains one or more the disclosed synthetic compound(s), the total amount of the synthetic compound(s) collectively in the pharmaceutical formulation that is effective to treat a viral infection (such as a respiratory viral infection, for example, SARS-CoV-2 infection) or inflammatory disease or disorder can be at least 0.001 wt %, at least 0.005 wt %, at least 0.01 wt %, at least 0.05 wt %, at least 0.1 wt %, in a range from 0.001 wt % to 50 wt %, from 0.005 wt % to 50 wt %, from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.001 wt % to 10 wt %, from 0.005 wt % to 10 wt %, from 0.001 wt % to 1 wt %, from 0.005 wt % to 5 wt %, from 0.1 wt % to 20 wt %, from 0.01 wt % to 10 wt %, from 0.05 wt % to 10 wt %, or from 0.1 wt % to 10 wt %.

For example, when the pharmaceutical formulation contains M13, or one or more the disclosed synthetic compound(s), the amount of the M13, or the total amount of the synthetic compound(s) collectively, in the pharmaceutical formulation that is effective to treat a viral infection (such as a respiratory viral infection, for example, SARS-CoV-2 infection) can be at least 0.001 wt %, at least 0.005 wt %, at least 0.01 wt %, at least 0.05 wt %, at least 0.1 wt %, in a range from 0.001 wt % to 50 wt %, from 0.005 wt % to 50 wt %, from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.001 wt % to 10 wt %, from 0.005 wt % to 10 wt %, from 0.001 wt % to 1 wt %, from 0.005 wt % to 5 wt %, from 0.1 wt % to 20 wt %, from 0.01 wt % to 10 wt %, from 0.05 wt % to 10 wt %, or from 0.1 wt % to 10 wt %.

For example, when the pharmaceutical formulation contains two or more of M13, and the disclosed synthetic compound(s), the total amount of the M13, and/or synthetic compound(s) collectively, in the pharmaceutical formulation that is effective to treat a viral infection (such as a respiratory viral infection, for example, SARS-CoV-2 infection) can be at least 0.001 wt %, at least 0.005 wt %, at least 0.01 wt %, at least 0.05 wt %, at least 0.1 wt %, in a range from 0.001 wt % to 50 wt %, from 0.005 wt % to 50 wt %, from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.001 wt % to 10 wt %, from 0.005 wt % to 10 wt %, from 0.001 wt % to 1 wt %, from 0.005 wt % to 5 wt %, from 0.1 wt % to 20 wt %, from 0.01 wt % to 10 wt %, from 0.05 wt % to 10 wt %, or from 0.1 wt % to 10 wt %.

In some forms, the pharmaceutical formulation containing M13, and/or the disclosed synthetic compound(s) can be provided in a unit dosage form. The dosage of each of M13, and the disclosed synthetic compound(s), when present in the pharmaceutical formulation in the unit dosage form, can be in a range from about 0.002 mg to about 1 mg, in a range from about 0.006 mg to about 0.6 mg, in a range from about 0.01 mg to about 0.4 mg, in a range from about 0.02 mg to about 0.3 mg, or in a range from about 0.01 mg to about 0.2 mg.

### C. Kits

The disclosed synthetic compounds, reagents, formulations, and other materials can be packaged together in any suitable combination as a kit useful for performing, or aiding in the performance of, the methods. It is useful if the components in a given kit are designed and adapted for use together in the method.

For example, kits including the pharmaceutical formulations of the synthetic compounds for administration to a subject, may include a pre-measured dosage of the composition in a sterile needle, ampule, tube, container, or other suitable vessel. The kits may include instructions for dosages and dosing regimens. In some forms, the compositions are lyophilized. The kit may further include agents (e.g., saline, a buffered solution) and instructions to form a formulation for administration. The instructions may specify suitable storage conditions for the kit and components thereof.

### III. Methods of Making

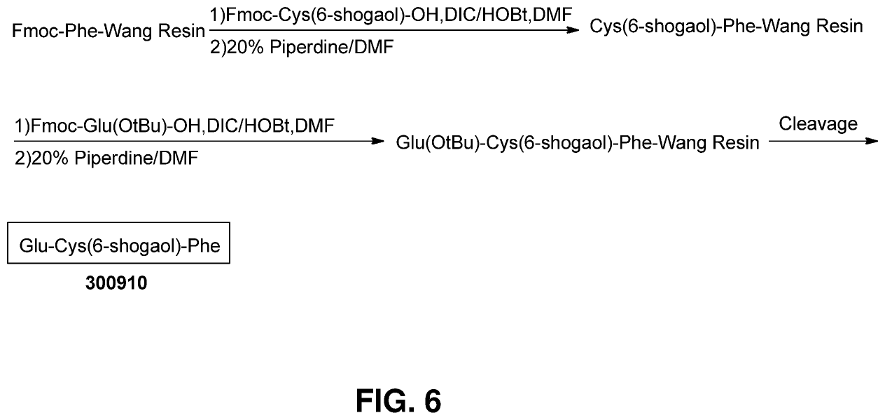

The compounds can be synthesized using methods known in the art of organic synthesis, such as methods that use 6-shogaol or an analog thereof and a three amino acid sequence containing Cys in the middle as the starting material in a suitable solvent medium to covalently attach 6-shogaol to the Cys through reaction between an unsaturated carbon-carbon bond and the thiol group of the Cys. FIG. 6 illustrates an exemplary scheme for the synthesis of the synthetic compounds Analogs of M13 can also be synthesized using methods known in the art of organic synthesis, such as those described in Chen, Huadong; Soroka, Dominique N.; Hu, Yuhui; Chen, Xiaoxin; Sang, Shengmin; Molecular Nutrition & Food Research (2013), 57(3), 447-458.

More specific methods for synthesizing exemplary compounds, are described in the Example below.

### IV. Methods of Use

### A. Methods of Using the Synthetic M13 Analog (s)

It has been established that synthetic M13 analog (s) (herein referred to as “the synthetic compounds”) can be administered to a subject to treat one or more symptoms of a microbial infection, e.g., a coronavirus infection. It is further established that the synthetic compounds can be administered to a subject in need thereof, to reduce an inflammatory response, e.g., an inflammatory response associated with a viral infection or an inflammatory disease or disorder. Therefore, methods of using the disclosed synthetic compounds and pharmaceutical formulations containing the synthetic compounds are provided.

The synthetic compounds and formulations thereof, can be used for treating a variety of diseases and disorders such as microbial infections, inflammatory diseases or disorders, and cancers. It will be appreciated that the disclosed methods can be methods of treatment of microbial infections, inflammatory diseases or disorders, and cancers and the treatment of the symptoms and conditions associated with microbial infections, inflammatory diseases or disorders, and cancers. For example, the synthetic compounds can be administered to a subject in need thereof to prevent or treat a microbial infection in the subject, such as indicated by the improvement or relief of one or more symptoms associated with the microbial infection in the subject.

“Treatment”, as used herein, refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The terms “high,” “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

The term “inhibit” means to reduce or decrease in activity or expression. This can be a complete inhibition of activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “in need of treatment” as used herein refers to a judgment made by a caregiver (e.g., physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that include the knowledge that the subject is ill, or will be ill, as the result of a condition that is treatable by the disclosed compounds and compositions thereof.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

### 1. Treating Microbial Infections

Methods of using the synthetic compounds to treat a microbial infection or a disease associated with a microbial infection in a subject in need thereof are disclosed. The microbial infection or disease associated with a microbial infection can be local or systemic in the subject.

Generally, the method for treating a microbial infection, or treating one or more symptoms associated with a microbial infection in a subject in need thereof, includes administering to the subject a pharmaceutical formulation containing one or more of the synthetic compounds disclosed herein. The pharmaceutical formulation can be administered in an effective amount to treat the microbial infection, or treat one or more symptoms associated with the microbial in the subject, as shown by one or more known clinical and/or biochemical measurements, such as reduction of bronchiolitis, alveolitis, pneumonia, meningitis, sepsis, and vasculitis; bronchiolar epithelial cell death and desquamation, alveolar space mononuclear cell infiltration, protein rich fluid exudation, alveolar hemorrhage, damage to alveolar structure, pulmonary blood vessel wall inflammation and endothelium infiltration, focal alveolar septal congestion and perivascular infiltration, and lower alveolar space immune cells, or a combination thereof.

In some forms, pharmaceutical formulation containing the synthetic compounds can be used to treat a subject with a viral infection or a subject at risk of developing one or more symptoms associated with a viral infection such as bronchitis, sinusitis, coughing, sneezing, and ear infections. In some forms, the pharmaceutical formulation containing one or more of the synthetic compounds can be used to treat a subject having an elevated risk of developing one or more severe symptoms associated with a viral infection, such as systemic inflammatory response syndrome, pneumonia, sepsis, or septic shock. In some forms, the methods of treatment with the pharmaceutical formulation containing one or more synthetic compounds are based on determining that the subject has one or more viral markers that are known in art to increase risk for the subject to develop moderate to severe symptoms associated with a viral infection. In one preferred form, the pharmaceutical formulation is used to treat a subject infected by an RNA virus, preferably an RNA virus of the family Coronaviridae. More preferably, the pharmaceutical formulation is used to treat a subject infected by a coronavirus.

i. Coronaviruses and SARS-CoV-2

The coronaviruses (order Nidovirales, family Coronaviridae, and genus Coronavirus) are a diverse group of large, enveloped, positive-stranded RNA viruses that cause respiratory and enteric diseases in humans and other animals.

Coronaviruses typically have narrow host specificity and can cause severe disease in many animals, and several viruses, including infectious bronchitis virus, feline infectious peritonitis virus, and transmissible gastroenteritis virus, are significant veterinary pathogens. Human coronaviruses (HCoVs) are found in both group I (HCoV-229E) and group 2 (HCoV-OC43) and are historically responsible for ~30% of mild upper respiratory tract illnesses.

At ~30,000 nucleotides, their genome is the largest found in any of the RNA viruses. There are three groups of coronaviruses; groups 1 and 2 contain mammalian viruses, while group 3 contains only avian viruses. Within each group, coronaviruses are classified into distinct species by host range, antigenic relationships, and genomic organization. The genomic organization is typical of coronaviruses, with the characteristic gene order (5′-replicase [rep], spike [S], envelope [E], membrane [M], nucleocapsid [N]-3′) and short untranslated regions at both termini. The SARS-CoV rep gene, which includes approximately two-thirds of the genome, encodes two polyproteins (encoded by ORFla and ORF1b) that undergo co-translational proteolytic processing. There are four open reading frames (ORFs) downstream of rep that are predicted to encode the structural proteins, S, E, M, and N, which are common to all known coronaviruses.

In some forms, the coronavirus disease to be treated is COVID associated with SARS-CoV-2 betacoronavirus of the subgenus Sarbecovirus. SARS-CoV-2 viruses share approximately 79% genome sequence identity with the SARS-CoV virus identified in 2003. The genome organization of SARS-CoV-2 viruses is shared with other beta coronaviruses; six functional open reading frames (ORFs) are arranged in order from 5′ to 3′: replicase (ORFla/ORF1b), spike (S), envelope (E), membrane (M) and nucleocapsid (N). In addition, seven putative ORFs encoding accessory proteins are interspersed between the structural genes.

In some forms, the coronavirus is a variant of SARS-CoV-2, such as SARS-CoV-2 B.1.1.7 (Alpha variant), SARS-CoV-2 B.1.351 (Beta variant), SARS-CoV-2 P.1 (Gamma variant), SARS-CoV-2 B.1.617, SARS-CoV-2 B.1.617.1 (Kappa variant), SARS-CoV-2 B.1.621 (Mu variant), SARS-CoV-2 B.1.617.2 (Delta variant), SARS-CoV-2 B.1.617.3, or SARS-CoV-2 B.1.1.529 (Omicron variant). In some forms, the coronavirus can be a sub-variant of the SARS-CoV-2 B.1.1.7 (Alpha variant), a sub-variant of the SARS-CoV-2 B.1.351 (Beta variant), a sub-variant of the SARS-CoV-2 P.1 (Gamma variant), a sub-variant of the SARS-CoV-2 B.1.617, a sub-variant of the SARS-CoV-2 B.1.617.1 (Kappa variant), a sub-variant of the SARS-CoV-2 B.1.621 (Mu variant), a sub-variant of the SARS-CoV-2 B.1.617.2 (Delta variant), a sub-variant of the SARS-CoV-2 B.1.617.3, or a sub-variant of the SARS-CoV-2 B.1.1.529 (Omicron variant), or a sub-variant derived from a descendent lineage of one or more of the foregoing sub-variants. For example, when the SARS-CoV-2 variant is an Omicron variant, the Omicron sub-variant can be a BA.1 sub-variant, a BA.2 sub-variant, a BA.3 sub-variant, a BA.4 sub-variant, a BA.5 sub-variant, or a BA.1/BA.2 circulating recombinant sub-variant such as XE.

In some forms, when the pathogenic coronavirus is SARS-CoV-2, the SARS-CoV-2 variant can be a variant of the wild-type strain of the coronavirus. “Wild-type” as used herein, refers to the original strain of coronavirus considered to be the background strain of the coronavirus containing no major mutations.

Patients with SARS-CoV-2 infection can experience a range of clinical manifestations, from no symptoms to critical illness. In general, adults with SARS-CoV-2 infection can be grouped into the following severity of illness categories; however, the criteria for each category may overlap or vary across clinical guidelines and clinical trials, and a patient's clinical status may change over time.
(i) Asymptomatic or pre-symptomatic infection: individuals who test positive for SARS-CoV-2 using a virologic test (i.e., a nucleic acid amplification test or an antigen test) but who have no symptoms that are consistent with COVID-19.
(ii) Mild illness: individuals who have any of the various signs and symptoms of COVID-19 (e.g., fever, cough, sore throat, malaise, headache, muscle pain, nausea, vomiting, diarrhea, loss of taste and smell) but who do not have shortness of breath, dyspnea, or abnormal chest imaging.
(iii) Moderate Illness: Individuals who show evidence of lower respiratory disease during clinical assessment or imaging and who have an oxygen saturation (SpO2)≥94% on room air at sea level.
(iv) Severe illness: individuals who have SpO₂<94% on room air at sea level, a ratio of arterial partial pressure of oxygen to fraction of inspired oxygen (PaO2/FiO2)<300 mm Hg, a respiratory rate >30 breaths/min, or lung infiltrates >50%. These patients may experience rapid clinical deterioration. Oxygen therapy should be administered immediately using a nasal cannula or a high-flow oxygen device. If secondary bacterial pneumonia or sepsis is suspected, administer empiric antibiotics, re-evaluate the patient daily, and de-escalate or stop antibiotics if there is no evidence of bacterial infection.
(v) Critical illness: individuals who have acute respiratory distress syndrome, septic shock that may represent virus-induced distributive shock, cardiac dysfunction, an exaggerated inflammatory response, and/or exacerbation of underlying comorbidities. In addition to pulmonary disease, patients with critical illness may also experience cardiac, hepatic, renal, central nervous system, or thrombotic disease.

Patients with certain underlying comorbidities are at a higher risk of progressing to severe COVID-19. These comorbidities include being aged >65 years; having cardiovascular disease, chronic lung disease, sickle cell disease, diabetes, cancer, obesity, or chronic kidney disease; being pregnant; being a cigarette smoker; being a transplant recipient; and receiving immunosuppressive therapy.

In some cases, patients with COVID-19 may have additional infections that are noted when they present for care or that develop during the course of treatment. These coinfections may complicate treatment and recovery. Older patients or those with certain comorbidities or immunocompromising conditions may be at higher risk for these infections.

In some forms, the pharmaceutical formulation containing the synthetic 6-shogaol derivative (s) can be used to reduce the replication of SARS-CoV-2 variants that predispose the host to developing severe COVID. In further forms, the pharmaceutical formulation containing the synthetic 6-shogaol derivative (s) can be used to treat patients having an elevated risk of developing one or more symptoms associated with severe COVID-19 as a result of SARS-CoV-2 infection. In these cases, the patients carrying these SARS-CoV-2 variants are likely to develop one or more symptoms associated with severe illness, critical illness, and additional complications. The disclosed the pharmaceutical formulations containing the synthetic compounds can be used to treat a subject at risk of developing severe COVID in order to reduce the infection and replication of the virus at least by 50%, 60%, 70%, 80%, 90%, 95%, 97%, or 99%.

ii. SARS-CoV

In some forms, the pharmaceutical formulation containing the synthetic compounds can be used to reduce the replication of SARS-CoV variants that predispose the host to developing severe acute respiratory syndrome, otherwise known as SARS. SARS is caused by the SARS coronavirus, known as SARS CoV. SARS CoV is believed to be a strain of the coronavirus usually only found in small mammals that have mutated, thereby enabling it to infect humans.

A wide range of clinical manifestations are seen in patients with SARS from mild, moderate, to severe and rapidly progressive and fulminant disease. The estimated mean incubation period of SARS-CoV infection was 4.6 days (95% CI, 3.8-5.8 days) and 95% of illness onset occurred within 10 days. The mean time from symptom onset to hospitalization was between 2 and 8 days but was shorter toward the later phase of the epidemic. The mean time from symptom onset to need for invasive mechanical ventilation (IMV) and to death was 11 and 23.7 days, respectively.

The major clinical features of SARS are fever, rigors, chills, myalgia, dry cough, malaise, dyspnea, and headache. Sore throat, sputum production, rhinorrhea, nausea, vomiting, and dizziness are less common. Watery diarrhea was present in 40% to 70% of patients with SARS and tended to occur about 1 week after illness onset. SARS-CoV was detected in the serum and cerebrospinal fluid of 2 patients complicated by status epilepticus. Elderly patients with SARS-CoV infection might present with poor appetite, a decrease in general well-being, fracture as a result of fall, and confusion, but some elderly subjects might not be able to mount a febrile response. In contrast, SARS-CoV infection in children aged less than 12 years was generally mild, whereas infection in teenagers resembled that in adults. There was no mortality among young children and teenagers. SARS-CoV infection acquired during pregnancy carried a case fatality rate of 25% and was associated with a high incidence of spontaneous miscarriage, preterm delivery, and intrauterine growth retardation without perinatal SARS-CoV infection among the newborn infants.

Asymptomatic SARS-CoV infection was uncommon in 2003; a meta-analysis had shown overall sero-prevalence rates of 0.1% (95% CI, 0.02-0.18) for the general population and 0.23% for health care workers (95% CI, 0.02-0.45) in comparison with healthy blood donors, others from the general community, or patients without SARS-CoV infection recruited from the health care setting (0.16%, 95% CI, 0-0.37).

The clinical course of patients with SARS-CoV infection seemed to manifest in different stages. In the first week of illness of SARS-CoV infection, many patients presented with fever, dry cough, myalgia, and malaise that might improve despite the presence of lung consolidation and rising viral loads on serial samples. During the second week, many patients experienced recurrence of fever, worsening consolidation, and respiratory failure, while about 20% of patients progressed to ARDS requiring IMV. Peaking of viral load on day 10 of illness corresponded temporally to peaking of the extent of consolidation radiographically, and a maximal risk of nosocomial transmission, particularly to health care workers.

The disclosed pharmaceutical formulations containing one or more synthetic compounds may be administered to a patient infected with SARS-CoV and/or variants thereof, in order to reduce the infection and replication of the virus at least by 50%, 60%, 70%, 80%, 90%, 95%, 97%, or 99%, thereby ameliorating the symptoms described above.

iii. MERS-CoV

In some forms, the pharmaceutical formulation containing one or more synthetic compounds can be administered to a subject to reduce the replication of Middle East respiratory syndrome-related coronavirus (MERS-CoV) variants that predispose the host to developing Middle East Respiratory Syndrome (MERS). MERS-CoV is a coronavirus believed to be originally from bats. However, humans are typically infected from camels, either during direct contact or indirectly. Spread between humans typically requires close contact with an infected person. As of 2021, there is no specific vaccine or treatment for the disease, although attempts are being made.

The virus MERS-CoV is a member of the beta group of coronavirus, Betacoronavirus, lineage C. MERS-CoV genomes are phylogenetically classified into two clades, clade A and B. The earliest cases were of clade A clusters, while the majority of more recent cases are of the genetically distinct clade B. MERS-CoV is closely related to the Tylonycteris bat coronavirus HKU4 and Pipistrellus bat coronavirus HKU5.

The specific exposures that lead to sporadic MERS-CoV infections are unknown, therefore it is challenging to estimate the incubation period in primary cases. However, based on data from cases of human-to-human transmission, the incubation period is a median of 5-7 days, with a range of 2-14 days (median 5.2 days [95% CI 1.9-14.7]). Immunocompromised patients can present with longer incubation periods of up to 20 days.

The clinical presentation of patients infected with MERS-CoV ranges from asymptomatic or mild upper respiratory illness to rapidly progressive pneumonitis, respiratory failure, acute respiratory distress syndrome, septic shock, and multiorgan failure with fatal outcome. Some individuals remain asymptomatic whereas some go on to develop mild disease, which is why WHO classifies these individuals as mild or asymptomatic. Asymptomatic-to-mild infection rates of 25-50% have been reported. The signs and symptoms associated with MERS are non-specific, with or without multisystem involvement, and thus could be mistaken for other causes of respiratory tract or gastrointestinal illnesses. Therefore, the clinical diagnosis of MERS can be easily missed. Patients with MERS can typically present with fever, chills, rigors, headache, a non-productive cough, sore throat, arthralgia, and myalgia followed by dyspnea. Other associated symptoms include coryza, nausea, vomiting, dizziness, sputum production, diarrhea, and abdominal pain. Some patients with MERS can present with atypical symptoms of mild respiratory illness without a fever and a gastrointestinal illness that precedes the development of pneumonia. Neuromuscular manifestations include hypersomnolence, weakness, and tingling in the extremities similar to Guillain-Barre syndrome or virus-related sensory neuropathy. 68 Co-infection of MERS-CoV with other respiratory viruses (such as parainfluenza virus, rhinovirus, influenza A or B virus, respiratory syncytial virus, enteroviruses, and human metapneumovirus) and nosocomial bacterial infections has been reported in patients receiving intensive care.

The disclosed pharmaceutical formulations containing one or more synthetic compounds may be administered to a patient infected with MERS-CoV and/or variants thereof, in order to reduce the infection and replication of the virus at least by 50%, 60%, 70%, 80%, 90%, 95%, 97%, or 99%, thereby ameliorating the symptoms described above.

iv. Common Human Coronaviruses

Unlike the highly pathogenic SARS-CoV, MERS-CoV, and 2019-nCoV, the four so-called common HCoVs generally cause mild upper-respiratory tract illness and contribute to 15%-30% of cases of common colds in human adults, although severe and life-threatening lower respiratory tract infections can sometimes occur in infants, elderly people, or immunocompromised patients. In some forms, the pharmaceutical formulation containing one or more synthetic compounds may be administered to a patient in need thereof, to reduce the replication and ameliorate the pathology associated with one or more of the four common HCoVs.

Human coronavirus 229E (HCoV-229E) is a species of coronavirus which infects humans and bats. HCoV-229E is a member of the genus Alphacoronavirus and subgenus Duvinacovirus. It is an enveloped, positive-sense, single-stranded RNA virus which enters its host cell by binding to the APN receptor. HCoV-229E is associated with a range of respiratory symptoms, ranging from the common cold to high-morbidity outcomes such as pneumonia and bronchiolitis. However, such high morbidity outcomes are almost always seen in cases with co-infection with other respiratory pathogens. In some forms, HCoV-229E may cause acute respiratory distress syndrome (ARDS). HCoV-229E is also among the coronaviruses most frequently co-detected with other respiratory viruses, particularly with human respiratory syncytial virus (HRSV). The disclosed pharmaceutical formulations containing one or more synthetic compounds may be administered to a patient infected with HCoV-229E and/or variants thereof, in order to reduce the infection and replication of the virus at least by 50%, 60%, 70%, 80%, 90%, 95%, 97%, or 99%, thereby ameliorating the described symptoms.

Human coronavirus NL63 (HCoV-NL63) is a species of coronavirus, specifically a Setracovirus from among the Alphacoronavirus genus. The virus is an enveloped, positive-sense, single-stranded RNA virus which enters its host cell by binding to ACE2. The virus is found primarily in young children, the elderly, and immunocompromised patients with acute respiratory illness. It also has a seasonal association in temperate climates. The evolution of HCoV-NL63 appears to have involved recombination between an ancestral NL63-like virus circulating in African Triaenops afer bats and a CoV 229E-like virus circulating in Hipposideros bats. Recombinant viruses can arise when two viral genomes are present in the same host cell. The first cases of the infection with HCoV-NL63 were found in young children with severe lower respiratory tract infections admitted to hospitals. While the clinical presentation of the virus can be severe, it has also been found in mild cases of respiratory infection. The comorbidity of HCoV-NL63 with other respiratory infections, has made the specific symptoms of the virus difficult to pinpoint. A study of clinical symptoms in HCoV-NL63 patients without secondary infection, reported the most common symptoms to be fever, cough, rhinitis, sore throat, hoarseness, bronchitis, bronchiolitis, pneumonia, and croup. An early study investigating children with lower respiratory tract illness, found that HCoV-NL63 was more commonly found in outpatients than hospitalized patients, suggesting that it is a common cold virus similar to HCoV-229E and HCoV-OC43, which generally cause less severe symptoms. The disclosed pharmaceutical formulations containing one or more synthetic compounds may be administered to a patient infected with HCoV-NL63 and/or variants thereof, in order to reduce the infection and replication of the virus at least by 50%, 60%, 70%, 80%, 90%, 95%, 97%, or 99%, thereby ameliorating the described symptoms.

Human coronavirus OC43 (HCoV-OC43) is a member of the species Betacoronavirus 1, which infects humans and cattle. The infecting coronavirus is an enveloped, positive-sense, single-stranded RNA virus that enters its host cell by binding to the N-acetyl-9-O-acetylneuraminic acid receptor. Four HCoV-OC43 genotypes (A to D) have been identified, with genotype D most likely arising from genetic recombination. The complete genome sequencing of genotypes C and D and boot scan analysis shows recombination events between genotypes B and C in the generation of genotype D. Of 29 viral variants identified, none belong to the more ancient genotype A. Symptoms of an infection with HCoV-OC43 are as described for HCoV-229E and HCoV-NL63. The disclosed pharmaceutical formulations containing one or more synthetic compounds may be administered to a patient infected with HCoV-OC43 and/or variants thereof, in order to reduce the infection and replication of the virus at least by 50%, 60%, 70%, 80%, 90%, 95%, 97%, or 99%, thereby ameliorating the above-described symptoms.

Human coronavirus HKU1 (HCoV-HKU1) is an enveloped, positive-sense, single-stranded RNA virus which like the OC43 virus, enters its host cell by binding to the N-acetyl-9-O-acetylneuraminic acid receptor. HCoV-HKU1 has the Hemagglutinin esterase (HE) gene, which distinguishes it as a member of the genus Betacoronavirus and subgenus Embecovirus. Symptoms of an infection with HCoV-HKU1 are as described for HCoV-229E and HCoV-NL63. The disclosed pharmaceutical formulations containing one or more synthetic compounds may be administered to a patient infected with HCoV-HKU1 and/or variants thereof, in order to reduce the infection and replication of the virus at least by 50%, 60%, 70%, 80%, 90%, 95%, 97%, or 99%, thereby ameliorating the above-described symptoms.

v. Non-Human Coronaviruses

The coronavirus infection to be treated may be caused by an alpha coronavirus or beta coronavirus that can infect a non-human mammal. In some forms, the alpha coronavirus can canine enteric coronavirus (CECoV), feline coronavirus (FCoV), porcine respiratory coronavirus (PRCV), porcine epidemic diarrhea virus (PEDV), or transmissible gastroenteritis virus (TGEV). In some forms, the alpha coronavirus can be a variant derived from rhinolophus bat coronavirus HKU2 (Bat-CoV HKU2) or miniopterus bat coronavirus HKU8 (Bat-CoV HKU8). In some forms, the beta coronavirus can be canine respiratory coronavirus (CRCoV), murine coronavirus (M-CoV), porcine hemagglutinating encephalomyelitis virus (PHEV), hedgehog coronavirus 1, bovine coronavirus (B-CoV), or equine coronavirus (E-CoV). In some forms, the beta coronavirus can be a variant derived from tylonycteris bat coronavirus HKU4 (Bat-CoV HKU4), pipistrellus bat coronavirus HKU5 (Bat-CoV HKU5), or rousettus bat coronavirus HKU9 (Bat-CoV HKU9)

The coronavirus infection to be treated may also be caused by a gamma coronavirus or a delta coronavirus. In some forms, the gamma coronavirus can be Avian Infectious Bronchitis (AIBV) or Beluga Whale CoV SW1. In some forms, the delta coronavirus can be Bulbul CoV HKU11 (BuCoV HKU11), Thrush CoV HKU12 (ThCoV HKU12), Munia CoV HKU13 (MunCoV HKU13), Porcine CoV HKU15 (PDCoV HKU15), White-eye CoV HKU16 (WECoV HKU16), Sparrow CoV HKU17 (SpCoV HKU17), Magpie Robin CoV HKU18 (MRCoV HKU18), Night heron CoV HKU19 (NHCoV HKU19), wigeon CoV HKU20 (WiCoV HKU20), Common moorhen CoV HKU21 (CMCoV HKU21), falcon CoV HKU27 (FalCoV UAE-HKU27), houbara bustard CoV HKU28 (HouCoV UAE-HKU28), pigeon CoV HKU29 (PiCoV UAE-HKU29), and quail CoV HKU30 (QuaCoV UAE-HKU30), which are the best characterized DCoV species. Delta coronaviruses are described in further detail in Vlasova et al. (2021) Frontiers in Veterinary Science, Vol. 10, doi: 10.3389/fvets.2020.626785. Non-human coronaviruses are described in further detail in Kenney et al. 2020 Veterinary Pathology, Vol. 58, Issue 3, pages 438-452, doi: 10.1177/0300985820980842; Alluwaimi et al. (2020) Frontiers in Veterinary Science, Vol. 7, Article number 582287, doi: 10.3389/fvets.2020.582287).

vi. Other Viruses

The synthetic compounds and pharmaceutical formulations thereof, can also be administered to a subject in need thereof, to treat symptoms and diseases associated with a variety of additional viral infections. For example, the synthetic compounds can be used to treat a subject infected with a virus from the families, Flaviviridae, Orthomyxoviridae, Filoviridae, Coronaviridae, and Paramyxoviridae.

In some forms, the pharmaceutical formulation containing synthetic compounds may be administered to a patient infected with a virus of the Flaviviridae family, for example, Yellow Fever, Hepatitis C virus, Dengue virus 1, Dengue virus 2, Dengue virus 3, and Dengue virus 4, Japanese encephalitis, West Nile viruses, and Zika virus.

In some forms, the pharmaceutical formulations containing one or more synthetic compounds may be administered to a patient infected with a virus of the Orthoryxoviridae family, for example, Influenza A virus such as HIN1, HiN2, H2N2, H3N1, H3N2, H3N8, H5N1, H5N2, H5N3, H5N8, H5N9, H7N1, H7N2, H7N3, H7N4, H7N7, H7N9, H9N2, and HION7; Influenza B virus such as B/Harbin/07/94, OR Influenza C virus such as C/JHB/2/66.

In some forms, the pharmaceutical formulations containing one or more synthetic compounds may be administered to a patient infected with a virus of the Filoviridae family, for example, Cuevavirus such as Lloviu virus; Dianlovirus such as Mĕnglá virus; Ebolavirus such as Bombali virus, Reston virus, Sudan virus, Tai Forest virus and Ebola virus; and Marburgvirus such as Marburg virus and Ravn virus.

In some forms, the pharmaceutical formulations containing synthetic compounds may be administered to a patient infected with a virus of the Paramyxoviridae family, for example, Pneumovirus such as respiratory syncytial virus; Morbillivirus such as measles virus/rubeola; Respirovirus such as para-influenza viruses 1 and 3; and Rubulavirus such as mumps virus and para-influenza viruses 2 and 4.

vii. Other Microbes

The synthetic compounds and pharmaceutical formulations thereof, can also be administered to a subject in need thereof, to treat symptoms and diseases associated with a variety of microbial infections such as bacterial infections, parasitic infections, and fungal infections.

In some forms, the pharmaceutical formulations containing one or more synthetic compounds may be administered to a subject in need thereof, to treat symptoms associated with a bacterial infection. Bacterial infections can originate from any bacteria including, but not limited to Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

In some forms, the pharmaceutical formulations containing one or more synthetic compounds may be administered to a patient, to treat symptoms associated with a parasitic infection. Non-limiting examples of parasites include, but are not limited to Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni.

### 2. Treating Inflammatory Diseases or Disorders

The synthetic compounds and formulations are suitable for preventing or treating an inflammatory disease or disorder, or treating or ameliorating one or more symptoms associated with an inflammatory disease or disorder in a subject in need thereof. Both acute and chronic inflammatory diseases or disorders can be treated by the disclosed methods. Examples of suitable inflammatory diseases or disorders and symptoms associated with the inflammatory diseases or disorders that can be treated by the disclosed method include, but are not limited to, asthma, chronic peptic ulcer, tuberculosis, rheumatoid arthritis, periodontitis, ulcerative colitis, Crohn's disease, sinusitis, active hepatitis, acute bronchitis, appendicitis, ingrown toenail, sore throat, and physical trauma or wound, and a combination thereof.

The method for treating an inflammatory disease or disorder in a subject in need of include administering to the subject, a therapeutically effective amount of a formulation containing one or more synthetic compounds to treat the inflammatory disease or disorder, for example, to reduce the severity or prevent one or more symptoms of the inflammatory disease or disorder. The step of administering an effective amount of the pharmaceutical formulation can be achieved in a single administration step or using multiple steps of administering the pharmaceutical formulation.

### 3. Treating Cancer in a Subject

In some forms, the synthetic compounds can be used in a method for treating cancer in a subject in need thereof. Generally, the method includes administering to the subject, a pharmaceutical formulation containing a therapeutically effective amount of one or more synthetic compounds described above, for example, to reduce one or more symptoms of the cancer. The administration step can occur one or more times.

A cancer in a patient refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. In some circumstances, cancer cells will be in the form of a tumor; such cells may exist locally within an animal, or circulate in the blood stream as independent cells, for example, leukemic cells. A tumor refers to all neoplastic cell growth and proliferation, whether malignant or benign, and all precancerous and cancerous cells and tissues. A solid tumor is an abnormal mass of tissue that generally does not contain cysts or liquid areas. A solid tumor may be in the brain, colon, breasts, prostate, liver, kidneys, lungs, esophagus, head and neck, ovaries, cervix, stomach, colon, rectum, bladder, uterus, testes, and pancreas, as non-limiting examples. In some embodiments, the solid tumor regresses or its growth is slowed or arrested after the solid tumor is treated with the presently disclosed methods. In other embodiments, the solid tumor is malignant. In some embodiments, the cancer includes Stage 0 cancer. In some embodiments, the cancer includes Stage I cancer. In some embodiments, the cancer includes Stage II cancer. In some embodiments, the cancer includes Stage III cancer. In some embodiments, the cancer includes Stage IV cancer. In some embodiments, the cancer is refractory and/or metastatic. For example, the cancer may be refractory to treatment with radiotherapy, chemotherapy or monotreatment with immunotherapy. Cancer includes newly diagnosed or recurrent cancers, including without limitation, colitis associated cancer (CAC), acute lymphoblastic leukemia, acute myelogenous leukemia, advanced soft tissue sarcoma, brain cancer, metastatic or aggressive breast cancer, breast carcinoma, bronchogenic carcinoma, choriocarcinoma, chronic myelocytic leukemia, colon carcinoma, colorectal carcinoma, Ewing's sarcoma, gastrointestinal tract carcinoma, glioma, glioblastoma multiforme, head and neck squamous cell carcinoma, hepatocellular carcinoma, Hodgkin's disease, intracranial ependymoblastoma, large bowel cancer, leukemia, liver cancer, lung carcinoma, Lewis lung carcinoma, lymphoma, malignant fibrous histiocytoma, a mammary tumor, melanoma, mesothelioma, neuroblastoma, osteosarcoma, ovarian cancer, pancreatic cancer, a pontine tumor, premenopausal breast cancer, prostate cancer, rhabdomyosarcoma, reticulum cell sarcoma, sarcoma, small cell lung cancer, a solid tumor, stomach cancer, testicular cancer, and uterine carcinoma. The subject can be a mammal, such as a human, a dog, a cat, a rat, a monkey, rabbits, guinea pigs, etc., that is in need of cancer treatment. In some forms, the subject can be exhibiting symptoms of or diagnosed with cancer. A subject in need of treatment includes a subject already diagnosed with a cancer and/or a subject prone to developing a cancer. In some forms, a subject is successfully “treated” for cancer according to the disclosed methods if the patient shows one or more of the following: a reduction in the number of or complete absence of cancer cells; a reduction in the tumor size; inhibition of or an absence of cancer cell infiltration into peripheral organs including the spread of cancer into soft tissue and bone; inhibition of or an absence of tumor metastasis; inhibition or an absence of tumor growth; relief of one or more symptoms associated with the specific cancer; reduced morbidity and mortality; and improvement in quality of life.

In some forms, the methods include administering to a subject with cancer, a formulation containing a therapeutically effective amount of the synthetic compounds to slow down, and/or halt progression of a cancer. For example, the amount of the synthetic compounds administered to the subject is effective to reduce tumor cell viability, slow or halt tumor growth, or to reduce tumor burden in the subject. In some forms, the methods of treating cancer include reducing the tumorigenicity of tumors e.g., by reducing the frequency of cancer stem cells in the tumor. In some forms, the methods can include contacting one or more cancer cells with an effective amount of the synthetic compounds, to decrease or inhibit the proliferation and/or viability of the cancer cells compared to untreated control cancer cells.

In some forms, the methods include administering to a subject with cancer, a formulation containing a therapeutically effective amount of the synthetic compounds to alter a measurable biochemical or physiological marker. For example, in the case of ulcerative colitis-associated cancer, the amount of the synthetic compounds administered to the subject can be effective to reduce the production, inhibit the activation, or inhibit a signaling pathway of PI3K/Akt/MTOR. In some forms, the amount of the synthetic compounds administered to the subject can be effective to reduce the expression of one or more pro-inflammatory cytokines and/or chemokines including but not limited to TNFα, IL-1β, and IL-6. In some forms, the amount of the synthetic compounds administered to the subject can be effective to downregulate the expression of one or more kinases including but not limited to PI3K, SPHK2, CDK6, TRKB and RIPK4 in the subject following treatment.

In some forms, the methods of treating cancer include treating one or more symptoms associated with cancer in a subject. For example, the synthetic compounds can be used in a method for prophylactic use i.e., prevention, delay in onset, diminution, eradication, or delay in exacerbation of signs or symptoms after onset, and prevention of cancer relapse. For prophylactic use, a therapeutically effective amount of the synthetic compounds and formulations or pharmaceutically acceptable salts thereof as described are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms. Prophylactic administration can be used, for example, in the chemopreventative treatment of subjects presenting precancerous lesions, those diagnosed with early-stage malignancies, and for subgroups with susceptibilities (e.g., family, racial, and/or occupational) to particular cancers.

### 4. Methods of Administration

The methods for reducing the replication of a viral infection, or methods for achieving a desired alleviation of viral-associated disease symptoms, include administering to an animal, such as a mammal, especially a human being, an effective amount of a combination of a pharmaceutical formulation containing one or more synthetic compounds and optionally one or more therapeutic, prophylactic or diagnostic agents, such as part of the same formulation, or administered separately and independently at the same time or at different times (i.e., administration of the one or more therapeutic, prophylactic or diagnostic agents, and the one or more synthetic compounds is separated by a finite period of time from each other). Therefore, the term “combination” or “combined” is used to refer to either concomitant, simultaneous, or sequential administration of the one or more synthetic compounds and one or more optional therapeutic, prophylactic or diagnostic agents. The combinations can be administered either concomitantly (e.g., as an admixture), separately but simultaneously (e.g., via separate intravenous lines into the same subject; one agent is given orally while the other agent is given by infusion or injection, etc.), or sequentially (e.g., one agent is given first followed by the second).

### 5. Effective Amounts

Formulations including one or more synthetic compounds and one or more therapeutic, prophylactic, and/or diagnostic agents typically include an effective amount of an admixture of the one or more synthetic compounds and one or more therapeutic, prophylactic, and/or diagnostic agents. Effective amounts of the combined synthetic compounds are provided herein. It will be appreciated that in some forms the effective amount of the synthetic compounds and one or more therapeutic, prophylactic, and/or diagnostic agents is different from the amount that would be effective for the one or more therapeutic, prophylactic, and/or diagnostic agents to achieve the same result when administered in the absence of the synthetic compounds.

When used for treating a viral infection or a disease associated with a viral infection in a subject, the amount of synthetic compounds present in a pharmaceutical dosage unit, or otherwise administered to a subject, can be the amount effective to reduce the production, inhibit the activation, or inhibit a signaling pathway of PI3K/Akt/MTOR. In some forms, the amount of synthetic compounds present in a pharmaceutical dosage unit, or otherwise administered to a subject, can be the amount effective to reduce coronavirus replication in small airways and alveoli of the lungs. Preferably, the amount of synthetic 6-shogaol derivatives (s) present in the pharmaceutical dosage unit, is administered to the subject in an amount effective to reduce coronavirus replication by 50% or more 24 hours following administration.

In some forms, the amount of synthetic compounds present in a pharmaceutical dosage unit, or otherwise administered to a subject, can be the amount effective to reduce the expression of one or more pro-inflammatory cytokines and/or chemokines including but not limited to TNFα, IL-1β, and IL-6. In some forms, the amount of synthetic compounds present in a pharmaceutical dosage unit, or otherwise administered to a subject, can be the amount effective to downregulate the expression of one or more kinases including but not limited to PI3K, SPHK2, CDK6, TRKB and RIPK4 in a subject following treatment.

In some forms, the pharmaceutical formulation containing one or more synthetic compounds is administered to a subject in need thereof, to deliver the synthetic 6-shogaol derivative in an amount between about 0.1 mg and about 500 mg, inclusive, preferably between about 0.5 mg and about 500 mg, inclusive, more preferably between about 1 mg and about 25 mg, inclusive, for example, 5 mg, 10 mg, and/or 15 mg per kg.

### 6. Dosage Regimens

Dosing regimens are dependent on the severity of the infection or disease/disorder and/or methods of administration, and is known to those skilled in the art. A therapeutically effective amount of the synthetic compounds used in the methods of treatment is typically sufficient to reduce or alleviate a microbial infection, inflammatory disease/disorder or cancer and symptoms thereof.

A dosage regimen of the pharmaceutical formulation containing one or more synthetic compounds and optionally one or more therapeutic, prophylactic, and/or diagnostic agents, can include one or multiple administrations of the pharmaceutical formulation.

In some forms, the pharmaceutical formulations are administered at least 1, 2, 3, 4, 5, 6, 7, 8, 9, 10, 11, 12, 13, 14, 15, 16, 17, 18, 19, 20, 21, 22, 23, 24 or 30 hours, or more than 30 hours, up to 36 or 48 hours prior to or after the detection of the virus in the patient. In other forms, the pharmaceutical formulations are administered at least 1, 2, 3, 4, 5, 6, 7, 8, 9, 10, 11, 12, 13, 14, 15, 16, 17, 18, 19, 20, 21, 22, 23, 24 or 30 hours, or more than 30 hours, up to 36 or 48 hours prior to or after administering a separate therapeutic, prophylactic, or diagnostic agent. In certain forms, additive or more than additive effects of the administration of the pharmaceutical formulation containing one or more synthetic compounds in combination with one or more therapeutic and/or prophylactic agent (s) is evident after one day, two days, three days, four days, five days, six days, one week, two weeks, three weeks, or more than three weeks following administration.

An effective amount of the pharmaceutical formulations and optionally one or more therapeutic and/or prophylactic agents can be administered as a single unit dosage (e.g., as dosage unit), or sub-therapeutic doses that are administered over a finite time interval. Such unit doses may be administered on a daily basis for a finite time period, such as up to 3 days, or up to 5 days, or up to 7 days, or up to 10 days, or up to 15 days or up to 20 days or up to 25 days, are all specifically contemplated.

### 7. Subjects to Be Treated

A subject in need of treatment is a subject having symptoms associated with a viral infection or a subject having or at risk of having a disease associated with a viral infection. Preferably, the subject in need of treatment is a subject having or at risk of having symptoms associated with a coronavirus infection. Exemplary diseases associated with coronavirus infections include respiratory diseases, such as coronavirus-induced pneumonia, coronavirus-induced bronchitis, acute respiratory distress syndrome (ARDS), acute lung injury (ALI), multisystem inflammatory syndrome in children (MIS-C), and/or multisystem inflammatory syndrome in adults (MIS-A). In some forms, the subject is a mammal, including, but not limited to, murines, simians, humans, mammalian farm animals and livestock, mammalian sport animals, and mammalian pets. Preferably, the subject is a human.

A subject having a coronavirus infection is a subject that has been exposed to a coronavirus and has acute or chronic detectable levels of the coronavirus in his/her body or has signs and symptoms associated with infection of the coronavirus. Methods of assessing and detecting coronavirus infections in a subject are known by those of ordinary skill in the art. A subject at risk of having a coronavirus infection is a subject that may be expected to come in contact with a coronavirus as described above. Examples of such subjects are medical workers or those traveling to parts of the world where the incidence of infection is high. In some forms, the subject is at an elevated risk of an infection because the subject has one or more risk factors to have an infection. Examples of risk factors to be infected and/or develop mild, moderate, and/or severe symptoms include immunosuppression, immunocompromised, age (advanced or very young), and surgery. The degree of risk of infection depends on the multitude and the severity or the magnitude of the risk factors that the subject has. Risk charts and prediction algorithms are available for assessing the risk of an infection in a subject based on the presence and severity of risk factors. Other methods of assessing the risk of infection in a subject are known by those of ordinary skill in the art. In some forms, the subject who is at an elevated risk of an infection may be an apparently healthy subject. An apparently healthy subject is a subject who has no signs or symptoms of disease.

The effect of the pharmaceutical formulations pharmaceutical formulations including one or more synthetic compounds can be compared to a control. Suitable controls are known in the art and include, for example, an untreated subject, or a placebo-treated subject. A typical control is a comparison of a condition or symptom of a subject prior to and after administration of the pharmaceutical formulations including one or more synthetic compounds. The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some forms, the symptom, pharmacologic, or physiologic indicator is measured in a subject prior to treatment, and again one or more times after treatment is initiated. In some forms, the control is a reference level, or average determined based on measuring the symptom, pharmacologic, or physiologic indicator in one or more subjects that do not have the disease or condition to be treated (e.g., healthy subjects). In some forms, the effect of the treatment is compared to a conventional treatment that is known the art.

Suitable control subjects are unvaccinated subjects, or subjects receiving the same amount of a therapeutic, prophylactic and/or diagnostic agent in the absence of pharmaceutical formulations containing one or more synthetic compounds.

### 8. Routes of Administration

The synthetic compounds and pharmaceutical formulations thereof can be administered in an amount sufficient to reduce the replication of a coronavirus in a subject and ameliorate symptoms associated with a coronavirus infection are typically administered according to methods known for administering vaccines to subjects.

In some forms, the synthetic compounds and pharmaceutical formulations thereof are administered parenterally. The phrases “parenteral administration” and “administered parenterally” are art-recognized terms, and include modes of administration other than enteral and topical administration, such as injections, and include without limitation intravenous, intramuscular, intrapleural, intravascular, intrapericardial, intraarterial, intrathecal, intratracheal, intranasal intracapsular, intraorbital, intracardiac, intradennal, intraperitoneal, transtracheal, subcutaneous, subcuticular, intraarticular, subcapsular, subarachnoid, intraspinal and intrastemal injection and infusion. The disclosed pharmaceutical formulations containing one or more synthetic compounds can be administered parenterally, for example, by subdural, intravenous, intrathecal, intraventricular, intraarterial, intra-amniotic, intraperitoneal, or subcutaneous routes. In preferred forms, the disclosed pharmaceutical formulations containing one or more synthetic compounds are administered via oral, intravenous, intranasal, intraperitoneal, intratracheal, or intrathecal administration.

The dosages or amounts of the synthetic compounds and formulations thereof described herein, are large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex, and extent of the disease in the subject and can be determined by one of skill in the art. The dosage can be adjusted by the individual physician based on the clinical condition of the subject involved. The dose, schedule of doses and route of administration can be varied.

### B. Methods of Using M13

It has been established that M13 can be administered to a subject to treat a microbial infection and symptoms thereof, e.g., a coronavirus infection. It is further established that M13 can be administered to a subject in need thereof, to treat cancer, e.g., by reducing the growth of cancerous intestinal cells and ameliorating symptoms ulcerative-colitis associated cancer such as reducing metabolic dysbiosis of the intestinal microbiota. Therefore, methods of using M13 and pharmaceutical formulations thereof are provided.

1. Treating Symptoms associated with a Microbial Infection

Generally, the method for preventing or treating a microbial infection in a subject includes administering to the subject, a pharmaceutical formulation containing a therapeutically effective amount of M13, or a combination thereof, for example, to reduce or prevent a microbial infection and/or one or more symptoms of the microbial infection. The administration step can occur one or more times.

Microbial infections that can be treated using M13 include but are not limited to viral infections, bacterial infections, parasitic infections, and fungal infections. Exemplary viruses, bacteria, parasites, and fungi are described in detail in Section (IV)(A) above. M13 and pharmaceutical formulations thereof, are particularly suitable for treating a viral infection such as a coronavirus infection. For example, M13 and pharmaceutical formulations thereof can be administered to a subject in need, to reduce the symptoms associated with infection of a SARS-CoV-2 virus.

Effective amounts of M13 contained in the pharmaceutical formulation depend on many factors, including the indication being treated, the route of administration, co administration of other therapeutic compositions, and the overall condition of the patient. For example, depending on the route of administration, a suitable dose may be calculated according to body weight, body surface areas or organ size. In some forms, the total amount of M13 in the pharmaceutical formulation (in unit dosage form) can be from 0.1 mg to 500 mg.

For example, if the unit dosage form contains an effective amount of the compounds to prevent or treat the viral infection in the subject, as indicated by the improvement and/or relief of one or more symptoms associated with the viral infection in the subject, then the method only requires a single administration step.

Alternatively, if the unit dosage form contains less than the needed effective amount of the compounds to prevent or treat the viral infection in the subject, then the method involves at least two steps of administering the pharmaceutical formulation, and optionally more than two steps of administering the pharmaceutical formulation to the subject until an effective amount of the synthetic metabolite and/or formulation is administered to the subject to prevent or treat the viral infection, as indicated by the improvement and/or relief of one or more symptoms associated with the viral infection in the subject. When multiple administration steps are needed to administer an effective amount of the compounds to the subject, each administration step may involve administering the same dosage or different dosages of the pharmaceutical formulation to the patient; and the administration step may be repeated one or more times for a period of time. In some forms, the administration step is repeated once, twice, or three times, per day, for a time period of one day, three days, one week, two weeks, or one month. For example, the administration step is repeated every 4 hours, every 6 hours, every 12 hours, or every day, optionally for a time period from 1 day to 1 month, from 1 day to 2 weeks, from 1 day to 1 week, or from 1 day to 3 days.

### 2. Treating Cancer in a Subject

M13, and formulations thereof, are particularly suitable for treating or ameliorating cancer and/or one or more symptoms associated with cancer in a subject. Non-limiting example 6 describe the use of an exemplary formulation containing M13 for the treatment of colitis-associated cancer.

Generally, the method for treating or ameliorating cancer in a subject includes administering to the subject, a pharmaceutical formulation containing a therapeutically effective amount of the M13, for example, to reduce tumor growth and/or the severity of one or more symptoms of the cancer. The methods of using M13 for treating or ameliorating cancer include the methods described in Section IV (A) (3) above. The administration step can occur one or more times.

In some forms, the M13, or a combination thereof is administered in an effective amount to reduce the number of cancer cells in the subject by at least 20%, at least 30%, at least 40%, at least 50%, at least 55%, at least 60%, at least 65%, at least 70%, at least 75%, at least 80%, at least 85%, or at least 90% in the subject compared to the number of cancer cells in the subject before treatment.

Cancers that can be treated using M13 include but are not limited to gastrointestinal cancers and/or tumors, gastric cancer, colitis-associated cancer. Exemplary cancers are described in detail in Section (IV)(A) above. Preferably, the cancer to be treated is colitis-associated cancer.

An effective amount of the M13 can be administered as a single unit dosage (e.g., as 10 mg per kg dosage unit), or sub-therapeutic doses that are administered over a finite time interval. Such unit doses may be administered on a daily basis for a finite time period, such as up to 3 days, or up to 5 days, or up to 7 days, or up to 10 days, or up to 15 days or up to 20 days or up to 25 days, are all specifically contemplated.

In some forms, pharmaceutical formulations containing M13 is administered by oral administration, parenteral administration, inhalation, mucosal, topical administration, or a combination thereof. In exemplary forms, the formulation containing M13 is delivered via oral administration for the treatment of colitis-associated cancer.

The disclosed compositions and methods can be further understood through the following numbered paragraphs.

1. A compound having the structure:

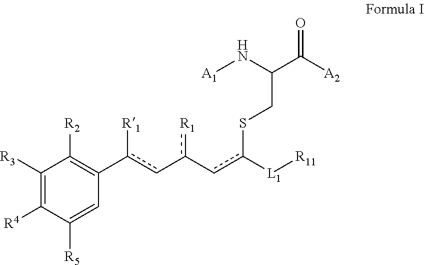

wherein:
   (i) [symbol] is a single or double bond;
   (ii) A₁ is an amino acid residue wherein the amino acid forming A₁ has a pH similar to glutamic acid;
   (iii) A₂ is an amino acid residue wherein the amino acid forming A₂ has a pH similar to glycine;
   (iv) R₁ is an oxygen, a hydroxyl, or —OR₆, and R₆ is an unsubstituted linear, branched, or cyclol C₁-C₆ alkyl;
   (v) R′₁ is a hydrogen, a hydroxyl, or —OR₈, and R₈ is an unsubstituted linear, branched, or cyclol C₁-C₆ alkyl;
   (vi) R₂-R₅ are independently a hydrogen, a hydroxyl, a halogen, an haloalkyl (e.g., —CF₃), or —OR₇, R₇ is an unsubstituted linear, branched, or cyclol C₁-C₆ alkyl, —C(═O)R₉, R₉ is hydrogen, an unsubstituted linear, branched, or cyclol C₁-C₆ alkyl, or —OR₁₀, and R₁₀ is hydrogen or an unsubstituted linear, branched, or cyclol C₁-C₆ alkyl;
   (vii) L₁ is a single bond or —(CH₂)ₘ—, m is an integer from 1 to 8;
   (viii) R₁₁ is a hydrogen, an unsubstituted or substituted linear C₁-C₁₀ alkyl, an unsubstituted or substituted branched C₃-C₁₀ alkyl, an unsubstituted or substituted C₃-C₁₀ cycloalkyl, or an unsubstituted or substituted C₅-C₁₂ aryl; and
   (ix) each substituent, when present, is independently a halogen, a hydroxyl, an haloalkyl, an unsubstituted linear, branched, or cyclol C₁-C₆ alkyl, an unsubstituted C₅-C₁₂ aryl, or —OR₁₂, R₁₂ is an unsubstituted linear, branched, or cyclol C₁-C₆ alkyl, and wherein the compound is not:

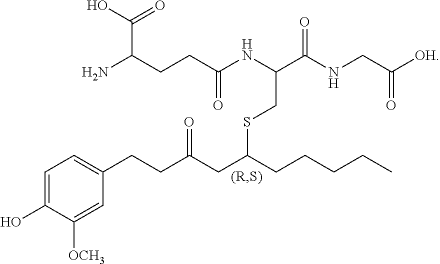 [SMILES: CCCCCC(CC(=O)CCc1ccc(O)c(OC)c1)SCC(NC(=O)CCC(N)C(=O)O)C(=O)NCC(=O)O]

2. The compound of paragraph 1, wherein the compound has the structure:

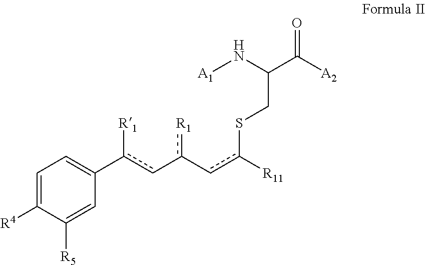

wherein:
   (i) [symbol] is a single or double bond;
   (ii) A₁ is an amino acid residue wherein the amino acid forming A₁ has a pH similar to glutamic acid;
   (iii) A₂ is an amino acid residue wherein the amino acid forming A₂ has a pH similar to glycine;
   (iv) R₁ is an oxygen, a hydroxyl, or —OR₆, and R₆ is an unsubstituted linear, branched, or cyclol C₁-C₆ alkyl;
   (v) R′₁ is a hydrogen, a hydroxyl, or —OR₈, and R₈ is an unsubstituted linear, branched, or cyclol C₁-C₆ alkyl;
   (vi) R₄ and R₅ are independently a hydrogen, a hydroxyl, or —OR₇, R₇ is an unsubstituted linear, branched, or cyclol C₁-C₆ alkyl; and
   (vii) R₁₁ is an unsubstituted linear C₅-C₉ alkyl, an unsubstituted branched C₃-C₈ alkyl, an unsubstituted C₃-C₈ cycloalkyl, an unsubstituted C₅-C₁₂ aryl, or a C₅-C₁₂ aryl substituted with a halogen, a hydroxyl, a haloalkyl, or —OR₁₂, R₁₂ is an unsubstituted linear, branched, or cyclol C₁-C₆ alkyl, or a combination thereof.

3. The compound of paragraph 1 or 2, wherein the compound has the structure:

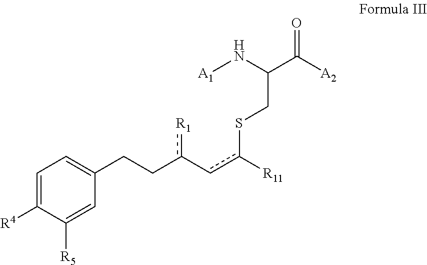

wherein:
   (i) [symbol] is a single or double bond;
   (ii) A₁ is an amino acid residue wherein the amino acid forming A₁ has a pH similar to glutamic acid;
   (iii) A₂ is an amino acid residue wherein the amino acid forming A₂ has a pH similar to glycine;
   (iv) R₁ is oxygen, hydroxyl, or —OR₆, and R₆ is an unsubstituted C₁-C₆ alkyl;
   (v) R₄ is a hydrogen or hydroxyl;
   (vi) R₅ is a hydrogen, a hydroxyl, or —OR₇, and R₇ is an unsubstituted C₁-C₆ alkyl; and
   (vii) R₁₁ is an unsubstituted branched C₃-C₆ alkyl, an unsubstituted C₃-C₆ cycloalkyl, an unsubstituted phenyl, or a phenyl substituted with a halogen.

4. The compound of paragraph 1 or 2, wherein the compound has the structure:

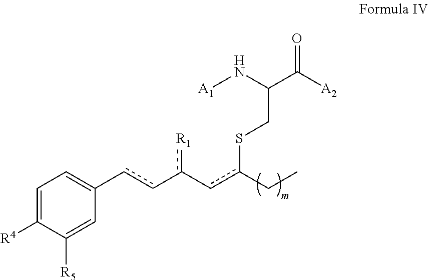

wherein:
   (i) [symbol] is a single or double bond;
   (ii) m is an integer from 4 to 8;
   (iii) A₁ is an amino acid residue wherein the amino acid forming A₁ has a pH similar to glutamic acid;
   (iv) A₂ is an amino acid residue wherein the amino acid forming A₂ has a pH similar to glycine;
   (v) R₁ is oxygen, hydroxyl, or —OR₆, and R₆ is an unsubstituted linear, branched, or cyclol C₁-C₆ alkyl;
   (vi) R₄ is a hydrogen or hydroxyl; and
   (vii) R₅ is a hydrogen, a hydroxyl, or —OR₇, and R₇ is an unsubstituted linear, branched, or cyclol C₁-C₆ alkyl.

5. The compound of paragraph 4, wherein the compound has the structure:

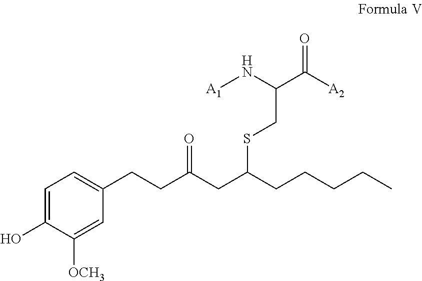

wherein A₁ is an amino acid residue wherein the amino acid forming A₁ has a pH similar to glutamic acid; and A₂ is an amino acid residue wherein the amino acid forming A₂ has a pH similar to glycine.

6. The compound of any one of paragraphs 1-5, wherein A₁ is a glutamic acid residue or an aspartic acid residue, or a synthetic derivative thereof, optionally an alpha-glutamic acid residue, a gamma-glutamic acid residue, an alpha-aspartic acid residue, or a beta-aspartic acid residue; and wherein A₂ is an alanine residue, a phenylalanine residue, a leucine residue, a methionine residue, a proline residue, a valine residue, a tryptophan residue, a tyrosine residue, or a glycine residue, or a synthetic derivative thereof.

7. The compound of any one of paragraphs 1-6, wherein the compound has the structure:

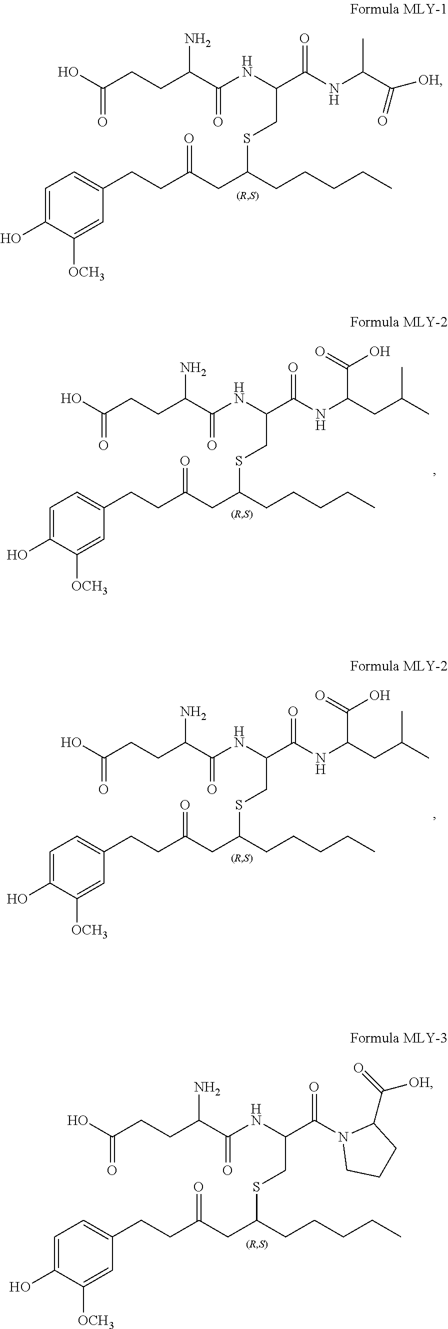 [SMILES: CCCCCC(CC(=O)CCc1ccc(O)c(OC)c1)SCC(NC(=O)C(N)CCC(=O)O)C(=O)NC(C)C(=O)O ; CCCCCC(CC(=O)CCc1ccc(O)c(OC)c1)SCC(NC(=O)C(N)CCC(=O)O)C(=O)NC(CC(C)C)C(=O)O ; CCCCCC(CC(=O)CCc1ccc(O)c(OC)c1)SCC(NC(=O)C(N)CCC(=O)O)C(=O)NC(CC(C)C)C(=O)O ; CCCCCC(CC(=O)CCc1ccc(O)c(OC)c1)SCC(NC(=O)C(N)CCC(=O)O)C(=O)N1CCCC1C(=O)O]

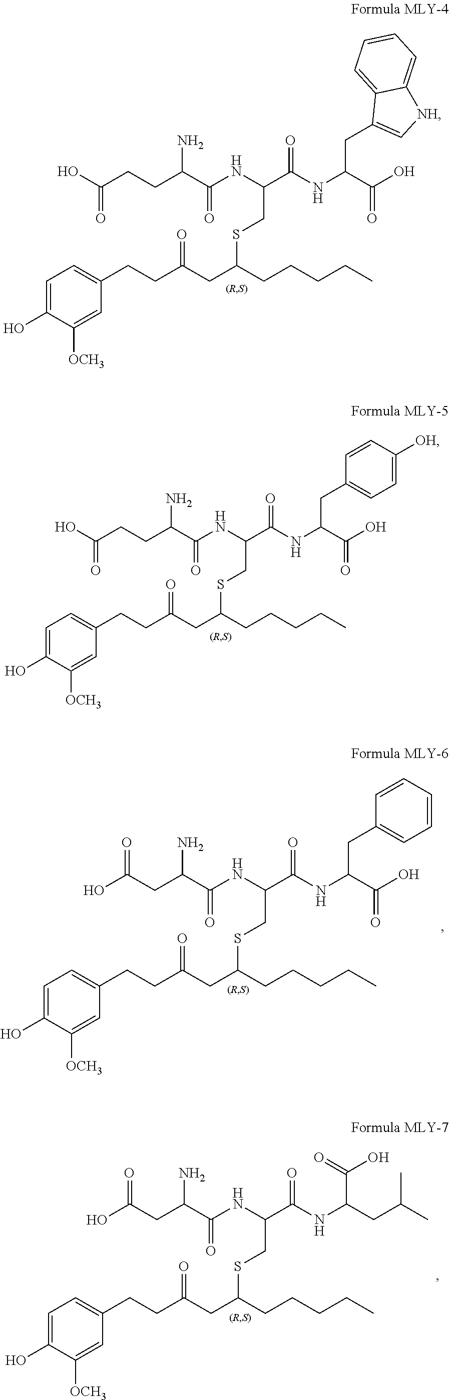 [SMILES: CCCCCC(CC(=O)CCc1ccc(O)c(OC)c1)SCC(NC(=O)C(N)CCC(=O)O)C(=O)NC(Cc1c[nH]c2ccccc12)C(=O)O ; CCCCCC(CC(=O)CCc1ccc(O)c(OC)c1)SCC(NC(=O)C(N)CCC(=O)O)C(=O)NC(Cc1ccc(O)cc1)C(=O)O ; CCCCCC(CC(=O)CCc1ccc(O)c(OC)c1)SCC(NC(=O)C(N)CC(=O)O)C(=O)NC(Cc1ccccc1)C(=O)O ; CCCCCC(CC(=O)CCc1ccc(O)c(OC)c1)SCC(NC(=O)C(N)CC(=O)O)C(=O)NC(CC(C)C)C(=O)O]

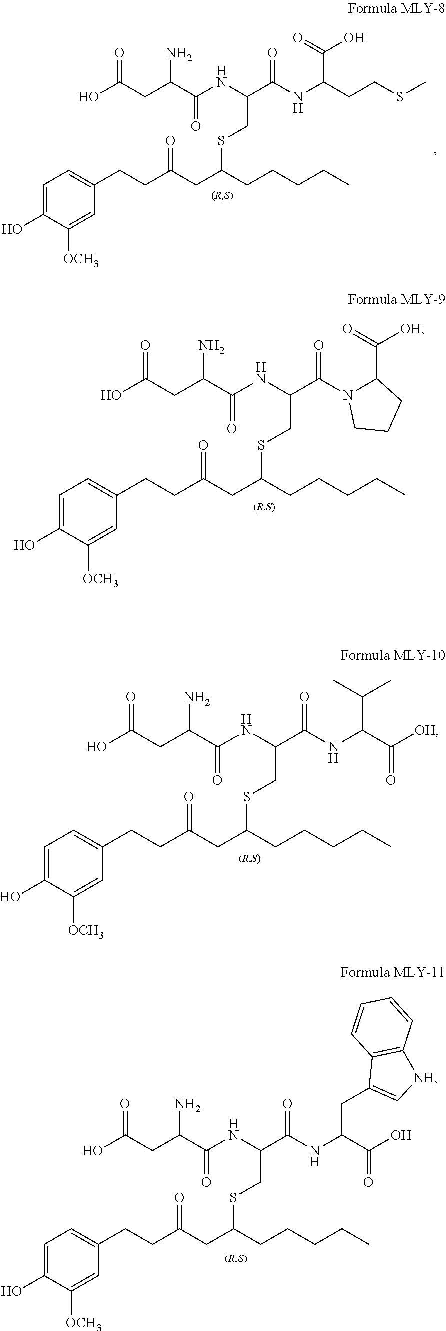 [SMILES: CCCCCC(CC(=O)CCc1ccc(O)c(OC)c1)SCC(NC(=O)C(N)CC(=O)O)C(=O)NC(CCSC)C(=O)O ; CCCCCC(CC(=O)CCc1ccc(O)c(OC)c1)SCC(NC(=O)C(N)CC(=O)O)C(=O)N1CCCC1C(=O)O ; CCCCCC(CC(=O)CCc1ccc(O)c(OC)c1)SCC(NC(=O)C(N)CC(=O)O)C(=O)NC(C(=O)O)C(C)C ; CCCCCC(CC(=O)CCc1ccc(O)c(OC)c1)SCC(NC(=O)C(N)CC(=O)O)C(=O)NC(Cc1c[nH]c2ccccc12)C(=O)O]

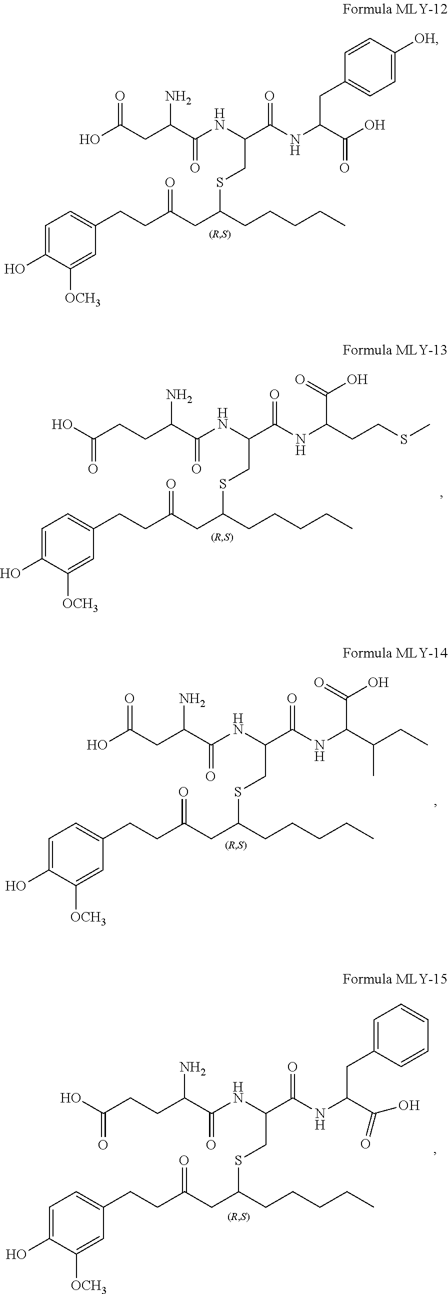 [SMILES: CCCCCC(CC(=O)CCc1ccc(O)c(OC)c1)SCC(NC(=O)C(N)CC(=O)O)C(=O)NC(Cc1ccc(O)cc1)C(=O)O ; CCCCCC(CC(=O)CCc1ccc(O)c(OC)c1)SCC(NC(=O)C(N)CCC(=O)O)C(=O)NC(CCSC)C(=O)O ; CCCCCC(CC(=O)CCc1ccc(O)c(OC)c1)SCC(NC(=O)C(N)CC(=O)O)C(=O)NC(C(=O)O)C(C)CC ; CCCCCC(CC(=O)CCc1ccc(O)c(OC)c1)SCC(NC(=O)C(N)CCC(=O)O)C(=O)NC(Cc1ccccc1)C(=O)O]

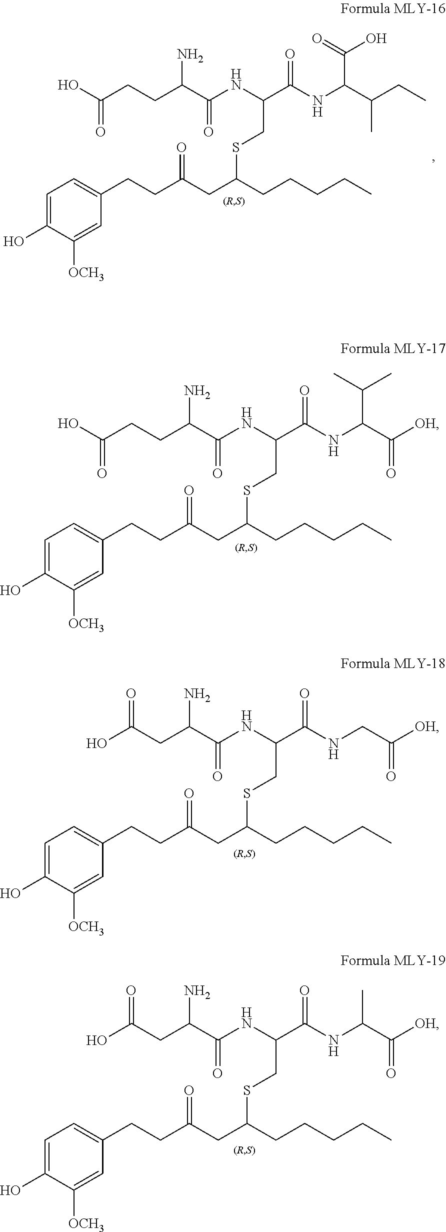 [SMILES: CCCCCC(CC(=O)CCc1ccc(O)c(OC)c1)SCC(NC(=O)C(N)CCC(=O)O)C(=O)NC(C(=O)O)C(C)CC ; CCCCCC(CC(=O)CCc1ccc(O)c(OC)c1)SCC(NC(=O)C(N)CCC(=O)O)C(=O)NC(C(=O)O)C(C)C ; CCCCCC(CC(=O)CCc1ccc(O)c(OC)c1)SCC(NC(=O)C(N)CC(=O)O)C(=O)NCC(=O)O ; CCCCCC(CC(=O)CCc1ccc(O)c(OC)c1)SCC(NC(=O)C(N)CC(=O)O)C(=O)NC(C)C(=O)O]

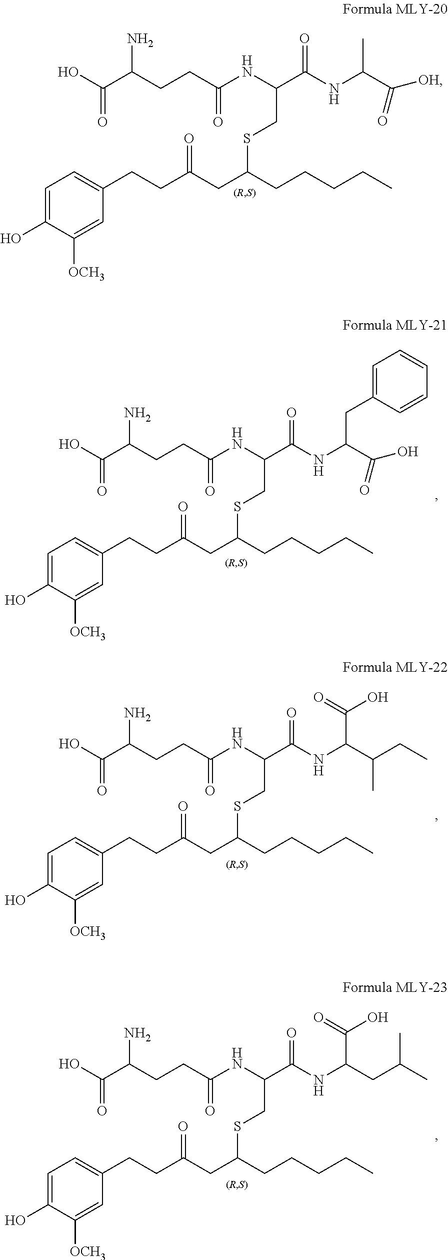 [SMILES: CCCCCC(CC(=O)CCc1ccc(O)c(OC)c1)SCC(NC(=O)CCC(N)C(=O)O)C(=O)NC(C)C(=O)O ; CCCCCC(CC(=O)CCc1ccc(O)c(OC)c1)SCC(NC(=O)CCC(N)C(=O)O)C(=O)NC(Cc1ccccc1)C(=O)O ; CCCCCC(CC(=O)CCc1ccc(O)c(OC)c1)SCC(NC(=O)CCC(N)C(=O)O)C(=O)NC(C(=O)O)C(C)CC ; CCCCCC(CC(=O)CCc1ccc(O)c(OC)c1)SCC(NC(=O)CCC(N)C(=O)O)C(=O)NC(CC(C)C)C(=O)O]

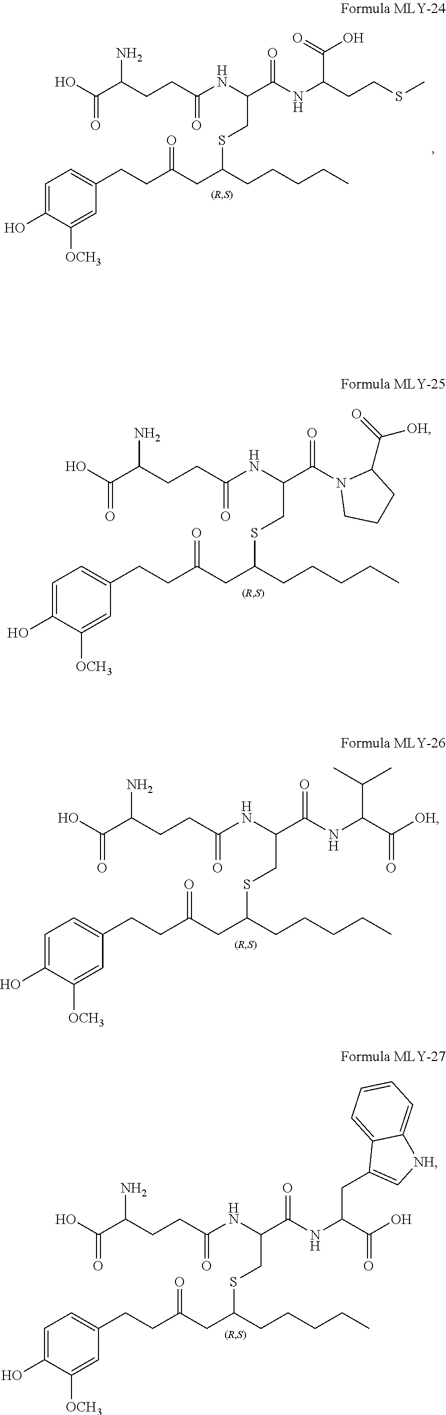 [SMILES: CCCCCC(CC(=O)CCc1ccc(O)c(OC)c1)SCC(NC(=O)CCC(N)C(=O)O)C(=O)NC(CCSC)C(=O)O ; CCCCCC(CC(=O)CCc1ccc(O)c(OC)c1)SCC(NC(=O)CCC(N)C(=O)O)C(=O)N1CCCC1C(=O)O ; CCCCCC(CC(=O)CCc1ccc(O)c(OC)c1)SCC(NC(=O)CCC(N)C(=O)O)C(=O)NC(C(=O)O)C(C)C ; CCCCCC(CC(=O)CCc1ccc(O)c(OC)c1)SCC(NC(=O)CCC(N)C(=O)O)C(=O)NC(Cc1c[nH]c2ccccc12)C(=O)O]

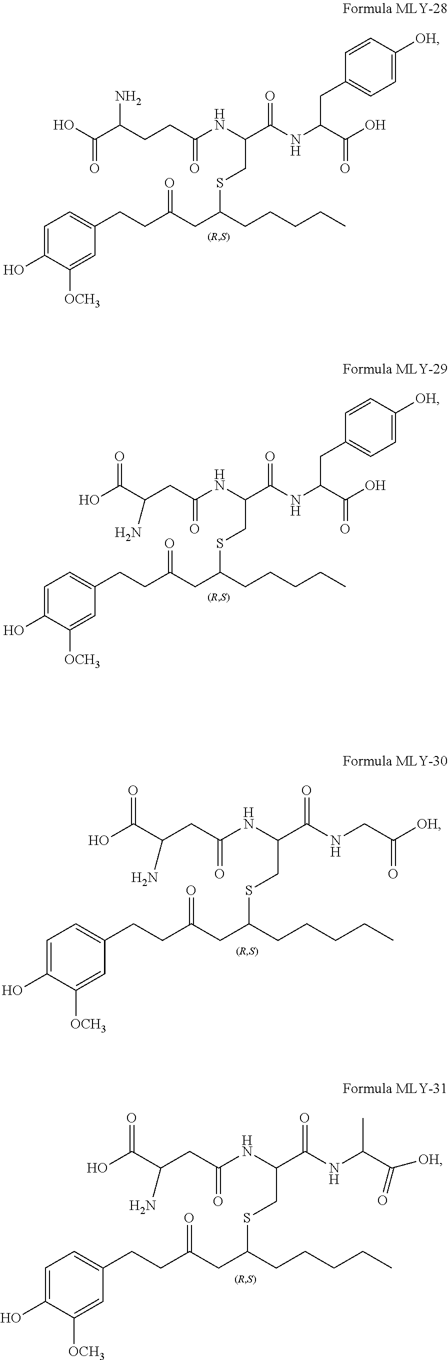 [SMILES: CCCCCC(CC(=O)CCc1ccc(O)c(OC)c1)SCC(NC(=O)CCC(N)C(=O)O)C(=O)NC(Cc1ccc(O)cc1)C(=O)O ; CCCCCC(CC(=O)CCc1ccc(O)c(OC)c1)SCC(NC(=O)CC(N)C(=O)O)C(=O)NC(Cc1ccc(O)cc1)C(=O)O ; CCCCCC(CC(=O)CCc1ccc(O)c(OC)c1)SCC(NC(=O)CC(N)C(=O)O)C(=O)NCC(=O)O ; CCCCCC(CC(=O)CCc1ccc(O)c(OC)c1)SCC(NC(=O)CC(N)C(=O)O)C(=O)NC(C)C(=O)O]

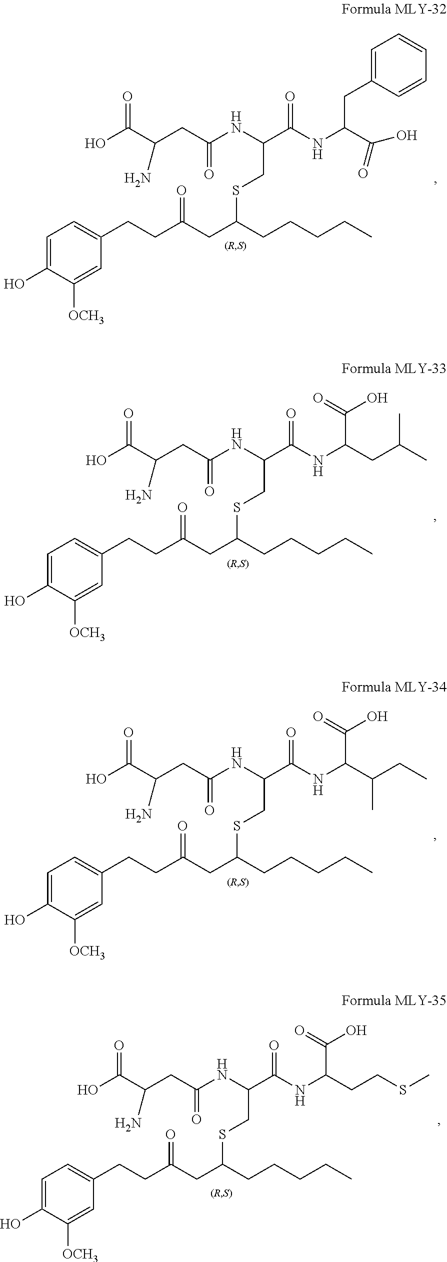 [SMILES: CCCCCC(CC(=O)CCc1ccc(O)c(OC)c1)SCC(NC(=O)CC(N)C(=O)O)C(=O)NC(Cc1ccccc1)C(=O)O ; CCCCCC(CC(=O)CCc1ccc(O)c(OC)c1)SCC(NC(=O)CC(N)C(=O)O)C(=O)NC(CC(C)C)C(=O)O ; CCCCCC(CC(=O)CCc1ccc(O)c(OC)c1)SCC(NC(=O)CC(N)C(=O)O)C(=O)NC(C(=O)O)C(C)CC ; CCCCCC(CC(=O)CCc1ccc(O)c(OC)c1)SCC(NC(=O)CC(N)C(=O)O)C(=O)NC(CCSC)C(=O)O]

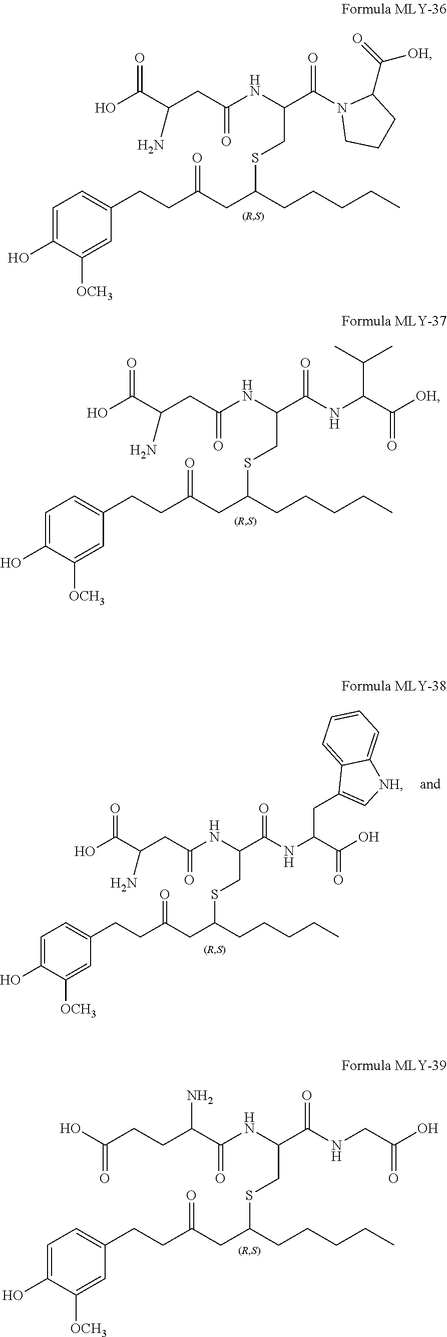 [SMILES: CCCCCC(CC(=O)CCc1ccc(O)c(OC)c1)SCC(NC(=O)CC(N)C(=O)O)C(=O)N1CCCC1C(=O)O ; CCCCCC(CC(=O)CCc1ccc(O)c(OC)c1)SCC(NC(=O)CC(N)C(=O)O)C(=O)NC(C(=O)O)C(C)C ; CCCCCC(CC(=O)CCc1ccc(O)c(OC)c1)SCC(NC(=O)CC(N)C(=O)O)C(=O)NC(Cc1c[nH]c2ccccc12)C(=O)O ; CCCCCC(CC(=O)CCc1ccc(O)c(OC)c1)SCC(NC(=O)C(N)CCC(=O)O)C(=O)NCC(=O)O]

8. A pharmaceutical formulation comprising one or more compounds of any one of paragraphs 1-7, and one or more pharmaceutically acceptable carriers and/or excipients.

9. The pharmaceutical formulation of paragraph 8, wherein the one or more compounds in the formulation are collectively in an amount effective, when administered to a subject, to reduce the expression in the subject of one or more pro-inflammatory cytokines and/or chemokines selected from the group consisting of TNFα, IL6, and IL1-β.

10. The pharmaceutical formulation of paragraph 8, wherein the one or more compounds in the formulation are each individually in an amount effective, when administered to a subject, to reduce the expression in the subject of one or more pro-inflammatory cytokines and/or chemokines selected from the group consisting of TNFα, IL6, and IL1-β.

11. The pharmaceutical formulation of any one of paragraphs 8-10, wherein the one or more compounds in the formulation are collectively in an amount effective, when administered to a subject, to increase the expression of IL-10 in the subject.

12. The pharmaceutical formulation of any one of paragraphs 8-10, wherein the one or more compounds in the formulation are each individually in an amount effective, when administered to a subject, to increase the expression of IL-10 in the subject.

13. The pharmaceutical formulation of any one of paragraphs 8-12, wherein the one or more compounds in the formulation are collectively in an amount effective, when administered to a subject, to reduce the expression in the subject of one or more kinases selected from the group consisting of PI3K, SPHK2, CDK6, TRKB, C-MER, EPHB2, RIPK4, RIPK5, and RIPK4.

14. The pharmaceutical formulation of any one of paragraphs 8-12, wherein the one or more compounds in the formulation are each individually in an amount effective, when administered to a subject, to reduce the expression in the subject of one or more kinases selected from the group consisting of PI3K, SPHK2, CDK6, TRKB, C-MER, EPHB2, RIPK4, RIPK5, and RIPK4.

15. The pharmaceutical formulation of any one of paragraphs 8-14, wherein the one or more compounds in the formulation are collectively in an amount effective, when administered to a subject, to treat a viral infection, a microbial infection, cancer, an inflammatory response, or a combination thereof, in the subject.

16. The pharmaceutical formulation of paragraph 15, wherein the one or more compounds in the formulation are each individually in an amount effective, when administered to a subject, to treat a viral infection, a microbial infection, cancer, an inflammatory response, or a combination thereof, in the subject.

17. The pharmaceutical formulation of paragraph 15, wherein the one or more compounds in the formulation are collectively in an amount effective, when administered to a subject, to ameliorate one or more symptoms of the viral infection, the microbial infection, the cancer, the inflammatory response, or of a combination thereof, in the subject.

18. The pharmaceutical formulation of paragraph 15, wherein the one or more compounds in the formulation are each individually in an amount effective, when administered to a subject, to ameliorate one or more symptoms of the viral infection, the microbial infection, the cancer, the inflammatory response, or of a combination thereof, in the subject.

19. The pharmaceutical formulation of paragraph 15, wherein the one or more compounds in the formulation are collectively in an amount effective, when administered to a subject, to ameliorate one or more symptoms of a disease associated with the viral infection, the microbial infection, the cancer, the inflammatory response, or a combination thereof, in the subject.

20. The pharmaceutical formulation of paragraph 15, wherein the one or more compounds in the formulation are each individually in an amount effective, when administered to a subject, to ameliorate one or more symptoms of a disease associated with the viral infection, the microbial infection, the cancer, the inflammatory response, or a combination thereof, in the subject.

21. The pharmaceutical formulation of any one of paragraphs 8-20, further comprising one or more additional active agents.

22. The pharmaceutical formulation of paragraph 21, wherein the one or more active agents are selected from the group consisting of a therapeutic agent, a prophylactic agent, a diagnostic agent, an anti-inflammatory agent, and an adjuvant.

23. The pharmaceutical formulation of any one of paragraphs 8-22, wherein the one or more compounds in the pharmaceutical formulation are collectively at a concentration of at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %.

24. The pharmaceutical formulation of any one of paragraphs 8-22, wherein the one or more compounds in the pharmaceutical formulation are each individually at a concentration of at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %.

25. A method of treating a subject, the method comprising:
administering to the subject the pharmaceutical formulation of any one of paragraphs 8-24, wherein the one of more compounds in the formulation are collectively in an amount effective to treat a viral infection, a microbial infection, cancer, an inflammatory response, or a combination thereof, in the subject,
wherein the administering is performed one or more times.

26. The method of paragraph 25, wherein the one or more compounds in the formulation are each individually in an amount effective to treat a viral infection, a microbial infection, cancer, an inflammatory response, or a combination thereof, in the subject.

27. The method of paragraph 25, wherein the one of more compounds in the formulation are collectively in an amount effective to ameliorate one or more symptoms of the viral infection, the microbial infection, the cancer, the inflammatory response, or of a combination thereof, in the subject.

28. The method of paragraph 25, wherein the one or more compounds in the formulation are each individually in an amount effective to ameliorate one or more symptoms of the viral infection, the microbial infection, the cancer, the inflammatory response, or of a combination thereof, in the subject.

29. The method of paragraph 25, wherein the one of more compounds in the formulation are collectively in an amount effective to ameliorate one or more symptoms of a disease associated with the viral infection, the microbial infection, the cancer, the inflammatory response, or a combination thereof, in the subject.

30. The method of paragraph 25, wherein the one or more compounds in the formulation are each individually in an amount effective to ameliorate one or more symptoms of a disease associated with the viral infection, the microbial infection, the cancer, the inflammatory response, or a combination thereof, in the subject.

31. The method of any one of paragraphs 25-30, wherein the subject has a viral infection.

32. The method of any one of paragraphs 25-31, wherein the viral infection is an infection by an RNA virus.

33. The method of paragraph 32, wherein the RNA virus is of a family selected from the group consisting of Flaviviridae, Orthomyxoviridae, Filoviridae, Coronaviridae, and Paramyxoviridae.

34. The method of any one of paragraphs 31-33, wherein the viral infection is an infection by a coronavirus.

35. The method of paragraph 34, wherein the coronavirus is an alpha coronavirus, a beta coronavirus, a gamma coronavirus, or a delta coronavirus.

36. The method of paragraph 34 or 35, wherein the coronavirus is selected from the group consisting of Human Coronavirus 229E (HCoV-229E), Human Coronavirus OC43 (HCoV-OC43), Human Coronavirus NL63 (HCoV-NL63), Human Coronavirus HKU1 (HCoV-HKU1), Middle East Respiratory Syndrome Coronavirus (MERS-CoV), Severe Acute Respiratory Syndrome Coronavirus 1 (SARS-CoV-1), and SARS-CoV-2.

37. The method of any one of paragraphs 34-36, wherein the coronavirus is a SARS-CoV-2 variant, wherein the SARS-CoV-2 variant is the alpha variant, beta variant, gamma variant, delta variant, epsilon variant, eta variant, iota variant, kappa variant, mu variant, omicron variant, zeta variant, 1.617.3 variant and/or lambda variant.

38. The method of paragraph 37, wherein the SARS-CoV-2 variant is a sub-variant of the alpha variant, beta variant, gamma variant, delta variant, epsilon variant, eta variant, iota variant, kappa variant, mu variant, omicron variant, zeta variant, 1.617.3 variant and/or lambda variant.

39. The method of any one of paragraphs 25-30, wherein the subject has a microbial infection.

40. The method of paragraph 39, wherein the microbial infection is caused by a microorganisms selected from the group consisting of a bacterium, a virus, a protozoan, and a fungus.

41. The method of any one of paragraphs 25-30, wherein the subject has cancer.

42. The method of any one of paragraphs 25-30, wherein the subject has an inflammatory response.

43. The method of any one of paragraphs 25-42, wherein the pharmaceutical formulation is administered by oral administration, intranasal administration, intratracheal administration, intramuscular administration, intravenous administration, intraperitoneal administration, or subcutaneous administration, or a combination thereof.

44. The method of any one of paragraphs 25-43, wherein the collective dosage of the compounds in the pharmaceutical formulation is from about 0.1 mg per kg to about 50 mg per kg, from about 0.5 mg to about 25 mg per kg, from about 1 mg to about 50 mg per kg of the subject.

45. The method of any one of paragraphs 25-44, wherein the individual dosage of each of the compounds in the pharmaceutical formulation is from about 0.1 mg per kg to about 50 mg per kg, from about 0.5 mg to about 25 mg per kg, from about 1 mg to about 50 mg per kg of the subject.

46. The method of any one of paragraphs 25-45, wherein the administration is repeated every 4 hours, every 6 hours, every 12 hours, or every day, optionally for a time period from 1 day to 1 month, from 1 day to 2 weeks, from 1 day to 1 week, or from 1 day to 3 days.

47. The method of any one of paragraphs 25-46, wherein the subject is a human.

48. The method of any one of paragraphs 25-47, wherein the subject is immunocompromised.

49. A pharmaceutical formulation comprising M13 and one or more pharmaceutically acceptable carriers and/or excipients, wherein the M13 in the formulation is in an amount effective, when administered to a subject, to treat a viral infection, a microbial infection, cancer, or a combination thereof, in the subject.

50. The pharmaceutical formulation of paragraph 49, further comprising one or more additional active agents.

51. The pharmaceutical formulation of paragraph 50, wherein the one or more active agents are selected from the group consisting of a therapeutic agent, a prophylactic agent, a diagnostic agent, an anti-inflammatory agent, and an adjuvant.

52. The pharmaceutical formulation of any one of paragraphs 49-51, wherein the M13 the formulation is in an amount effective, when administered to a subject, to reduce the expression in the subject of one or more pro-inflammatory cytokines and/or chemokines selected from the group comprising TNFα, IL6, and IL1-β.

53. The pharmaceutical formulation of any one of paragraphs 49-52, wherein the M13 in the formulation is in an amount effective, when administered to a subject, to increase the expression of IL-10 in the subject.

54. The pharmaceutical formulation of any one of paragraphs 49-53, wherein the M13 in the formulation is in an amount effective, when administered to a subject, to reduce the expression in the subject of one or more kinases selected from the group comprising PI3K, SPHK2, CDK6, TRKB, C-MER, EPHB2, RIPK4, RIPK5, and RIPK4.

55. The pharmaceutical formulation of any one of paragraphs 49-54, wherein the M13 in the pharmaceutical formulation is at a concentration of at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %.

56. A method of treating a subject, the method comprising:
administering to the subject the pharmaceutical formulation of any one of paragraphs 49-55, wherein the M13 in the formulation is in an amount effective to treat a viral infection, a microbial infection, cancer, or a combination thereof, in the subject,
wherein the administering is performed one or more times.

57. The method of paragraph 56, wherein the subject has a viral infection.

58. The method of paragraph 56 or 57, wherein the viral infection is an infection by an RNA virus.

59. The method of paragraph 58, wherein the RNA virus is of a family selected from the group consisting of Flaviviridae, Orthomyxoviridae, Filoviridae, Coronaviridae, and Paramyxoviridae.

60. The method of any one of paragraphs 57-59, wherein the viral infection is an infection by a coronavirus.

61. The method of paragraph 60, wherein the coronavirus is an alpha coronavirus, a beta coronavirus, a gamma coronavirus, or a delta coronavirus.

62. The method of paragraph 60 or 61, wherein the coronavirus is selected from the group consisting of Human Coronavirus 229E (HCoV-229E), Human Coronavirus OC43 (HCoV-OC43), Human Coronavirus NL63 (HCoV-NL63), Human Coronavirus HKU1 (HCoV-HKU1), Middle East Respiratory Syndrome Coronavirus (MERS-CoV), Severe Acute Respiratory Syndrome Coronavirus 1 (SARS-CoV-1), and SARS-CoV-2.

63. The method of any one of paragraphs 60-62, wherein the coronavirus is a SARS-CoV-2 variant, wherein the SARS-CoV-2 variant is the alpha variant, beta variant, gamma variant, delta variant, epsilon variant, eta variant, iota variant, kappa variant, mu variant, omicron variant, zeta variant, 1.617.3 variant and/or lambda variant.

64. The method of paragraph 63, wherein the SARS-CoV-2 variant is a sub-variant of the alpha variant, beta variant, gamma variant, delta variant, epsilon variant, eta variant, iota variant, kappa variant, mu variant, omicron variant, zeta variant, 1.617.3 variant and/or lambda variant.

65. The method of paragraph 56, wherein the subject has a microbial infection.

66. The method of paragraph 65, wherein the microbial infection is caused by a microorganisms selected from the group consisting of a bacterium, a virus, a protozoan, and a fungus.

67. The method of paragraph 56, wherein the subject has cancer.

68. The method of any one of paragraphs 56-67, wherein the pharmaceutical formulation is administered by oral administration, intranasal administration, intratracheal administration, intramuscular administration, intravenous administration, intraperitoneal administration, or subcutaneous administration, or a combination thereof.

69. The method of any one of paragraphs 56-68, wherein the dosage of the M13 in the pharmaceutical formulation is from about 0.1 mg per kg to about 50 mg per kg, from about 0.5 mg to about 25 mg per kg, from about 1 mg to about 50 mg per kg of the subject.

70. The method of any one of paragraphs 56-69, wherein the administration is repeated every 4 hours, every 6 hours, every 12 hours, or every day, optionally for a time period from 1 day to 1 month, from 1 day to 2 weeks, from 1 day to 1 week, or from 1 day to 3 days.

71. The method of any one of paragraphs 56-70, wherein the subject is a human.

### EXAMPLES

### Example 1: M-13 Demonstrates Good Biopharmaceutical Properties with Low Mutagenic Potential, Low In Vivo Toxicity and Low Immunomodulatory Effects on Unstimulated Lymphocytes

Coronavirus disease 2019 (COVID-19), caused by the SARS-CoV-2 virus, has greatly affected public health and medical service systems worldwide. In this context, anti-inflammatory drugs such as non-steroidal anti-inflammatory drugs (NSAIDs) and small molecule inhibitors able to modulate immune responses have been suggested to have an important role as potentially block various stages in COVID-19 life cycle (Kelleni et al. (2021), Biomed Pharmacother., Vol. 133: Article 110982; Chen et al. (2021), Int Immunopharmacol. Vol. 99: article 108027). The present study investigates the biopharmaceutical properties, mutagenic potential, and toxicity of M13.

### Methods

### M13 Synthesis

M13 was conjugated with a tripeptide (glutathione) via a semisynthetic approach. In brief, 6-shogaol (10 mg), reduced L-glutathione (30 mg), and NaHCO₃ (0.15 mg) were mixed in methanol/water (1.5 mL, 1:1, v/v). The mixture was shaken at room temperature for 3 hours, the pH was adjusted to 6.0 with a diluted acetic acid solution (0.1 M), and the mixture was extracted with n-butanol (BuOH; 5 mL×3). The organic layers were combined and concentrated under reduced pressure at 25° C. The residue was subjected to column chromatography and eluted with 90% ethanol in water.

### Assessment of the Mutagenic Potential of M13

The mutagenic potential of M13 was assessed using the Ames test. The Ames test, or bacterial reverse mutation test, introduced by Bruce Ames and his group in the early 1970s, is currently widely employed to assess the mutagenic potential of chemical compounds. In this biological assay, several strains of Salmonella ryphimurium carrying mutations in genes responsible for histidine synthesis are used, so these strains cannot produce this critical amino acid. The Ames test evaluates the mutagenesis potential of candidate substances by assessing whether mutations that return the bacteria to a “prototrophic” state (i.e., Reverse Mutation) can be induced, thereby allowing the bacteria to survive on the histidine-free medium. Briefly, the bacterial strains are spread on an agar plate with a small amount of histidine, which only allows the bacteria to grow for an initial time period and mutate. When the histidine is consumed, only bacteria that have mutated to have the ability for spontaneous histidine production will survive. A positive result indicates that the tested chemical is mutagenic, and may therefore act as a carcinogen, given that cancer is often linked to genetic mutation in oncological studies.

M13 at concentrations of 5000, 2500, 1250, 625, 312 and 156 g/plate and positive (NaN3; 2-nitrofluorene (NF) and 2-amonoanthracene) and negative (PBS) controls were tested on two tester strains; TA98 reverted from histidine dependence (auxotrophy) to histidine independence (prototrophy) by frameshift mutagens and tester TA100 reverted by mutagens that cause both frameshift and base pair substitution mutations. The colony numbers on the negative, positive control and M13 treated plates were determined by manual counting. The mean number of revertants per plate, the standard deviation, and the mutation factor (i.e., mean number of revertants on the test item plate/mean number of revertants on the vehicle plate) were calculated.

### In-Vivo Assessment of M13 Toxicity

A general toxicity study was performed by using maximum tolerated dose (MTD) of M13. Eight-week old mice were orally administered 1000 mg/kg or with PBS. On day one and day 7, mice were examined for weight loss and clinical scores were noted. Clinical signs were scored by assessing activity, appearance, and body condition for seven days.

### Results

### M-13 Demonstrates Good Biopharmaceutical Properties.

The main reason for failure in drug development is poor biopharmaceutical properties, which include low aqueous solubility, chemical instability. It was demonstrated that M13 (Molecular Weight, MW: 583.26) is very stable at various pH levels (pH 2 to pH 10). Also, it was found that M13 has high aqueous solubility as determined by: (i) the partition of M13 between and organic solvent (octanol) and aqueous buffer with a Log D7.4 of −1.75; (ii) the kinetic solubility, the maximum solubility of the fastest precipitating species of M13, which was found to be >200 M, and (iii) the thermodynamic (or equilibrium) solubility, and assessment of the solubility of M13 as a saturated solution in equilibrium, which was found to be 1390.93 M.

The pKa of a drug influences lipophilicity, solubility, protein binding, and permeability, which in turn directly affects pharmacokinetic (PK) characteristics such as absorption, distribution, metabolism, and excretion. It was found that M13 at physiological pH levels (pH 6 to pH 7) is not ionized, given the observations that M13 pka1=3.24; M13 pka2=9.12, and M13 pka3=10.42 as determined by UV metric at pH 2 to pH 12. Together these M13 physiochemical properties suggest that M13 exhibits a good balance between solubility and has low metabolic liability.

### M-13 Demonstrates Low Mutagenic Potential

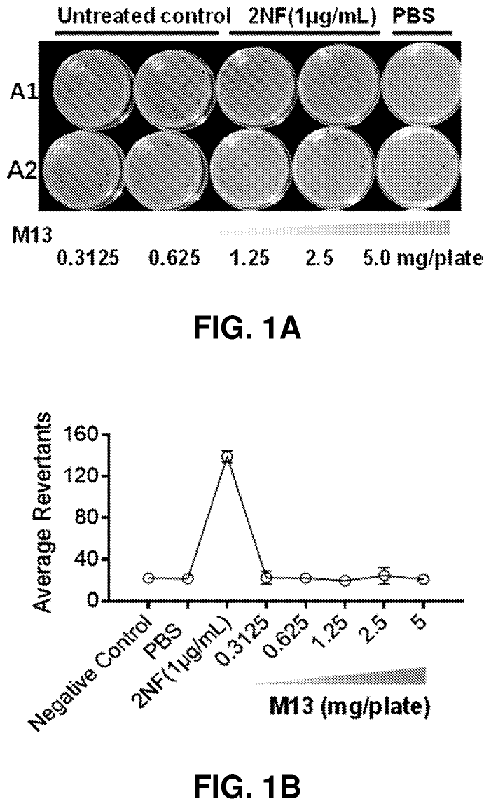

The mutagenic potential of M13 was assessed using the Ames test. The Ames test is a rapid and convenient assay to estimate the carcinogenic potential because standard assays in pre-clinical trials are time-consuming (~2 to 3 years) and are more expensive. A positive result on the Ames test indicates that the tested chemical is mutagenic and may therefore act as a carcinogen, since cancer is often linked to genetic mutation in an oncological study. It was found that M13 did not demonstrate an increase in revertant colony numbers, and the mutation factor was less than ~2.0 for all concentrations in all strains. These results are illustrated in FIGS. 1A and 1B which shows that M13 at all tested concentrations does not increase the number of revertants, thus, demonstrating the lack of mutagenic potential of M13. In FIG. 1A, A1 refers to the Tester strain TA98, under one of three conditions: untreated, treated with positive control (i.e., 2NF: 2-nitrofluorene), or PBS; and A₂ refers to the Tester strain TA98 treated with M13 at concentrations of 0.3125 mg/plate, 0.625 mg/plate, 1.25 mg/plate, 2.5 mg/plate, or 5 mg/plate. FIG. 1B shows the colony average revertant numbers on the negative, PBS positive control (2NF) and M13-treated plates as determined by manual counting.

Results from In-Vivo Assessment of M13 Toxicity

A general toxicity study was performed by using maximum tolerated dose (MTD) of M13. No significant changes were observed in weight loss and clinal signs in the treated mice during the seven days of observation (i.e., following oral administration of M13) when compared to the controlled mice (i.e., oral administration of PBS on day one). In addition, no significant differences were observed in white blood cell number, red blood cell number, hemoglobin levels, or parameters of hepatic or renal function in mice treated with M13 when compared to mice treated with PBS.

### Example 2: M13 Targets Kinases that Reduce the Inflammatory Response and Ameliorate the Development of COVID-19

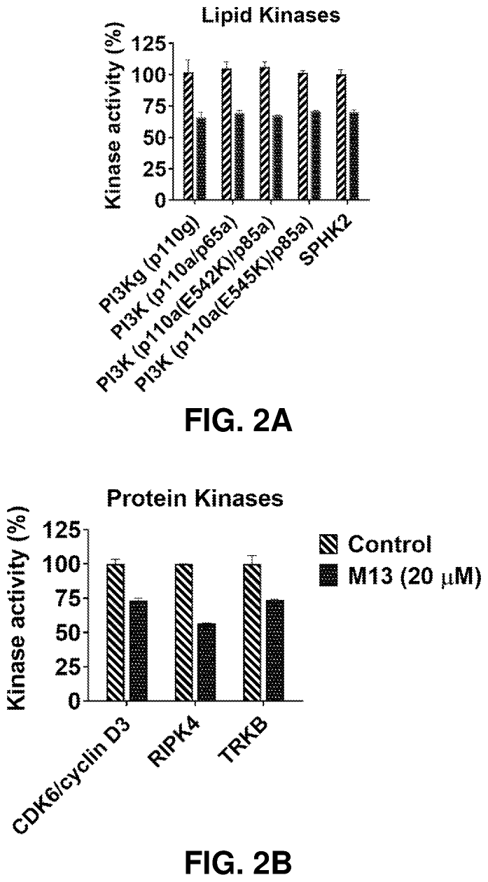

It was hypothesized that M13 may target kinases to reduce the inflammatory response. Determination and improvement of the selectivity of a compound by screening a large part of the kinome are of pivotal importance in the discovery of M13 signaling pathways for its anti-inflammatory activities. Radiometric assays were used based on the transfer of ³³P-labeled phosphate from ATP to the kinase substrates found in the 376 Wild Type Kinase Panel and 17 Lipid Kinase Panel (Reaction Biology) and a broad kinase selectivity screen for the inhibitory activity of M13 was performed. Briefly, radiometric assays are based on the transfer of 10 μM ³³P-labelled phosphate from ATP to the kinase substrate. It was found that 20 μM M13 inhibits enzyme activity relative to control of the lipid kinases PI3K, SPHK2 and the protein kinases CDK6, TRKB and RIPK4 by 30 to 45% (FIGS. 2A and 2B). FIGS. 2A and 2B show the targeted inhibitor effects of M13 on lipid kinases (FIG. 2A) and protein kinases (FIG. 2B).

Thus, as described in the studies of Examples 1 and 2, M13 demonstrates (i) excellent biopharmaceutical properties without mutagenic potential, without in vivo toxicity, and (ii) showed increased anti-inflammatory activities that could ameliorate the development of COVID-19.

### Example 3: Antiviral Activity and Cytotoxicity of M13 Against SARS-CoV-2 Materials

The aim of the present study was to determine the antiviral activity and the cytotoxicity of eight concentrations of three compounds against SARS-CoV-2.

### Methods

### Experimental Procedure

The antiviral activity of 8 dilutions of each of compounds 1-3 was explored by pre-incubation of cells with compounds 1, 2 or 3 for 60 minutes before addition of SARS-CoV-2 to the cells. Virus and formulations were then left on the cells for the entire duration of the experiment (24 hours). The cytotoxicity of the same range of concentrations of formulations was determined by MTT assay.

### Cell Plating

Cells were seeded in complete media at 8000 cells/100 μl/well in four plates: two for the cytotoxicity assay and two for the infectivity assay. After seeding, the plates were incubated at room temperature for 5 minutes for even distribution, and then at 37° C., 5% CO₂ until the following day.

### Formulation Dilutions

Formulations were prepared at twice the final concentration, and they were diluted to the final desired concentration by an equal volume of virus or media. The initial amounts weighted and the volume in which these were resuspended are recorded.

### Sample Preparation

The prepared stocks (0.5 mg/ml) were diluted in 800 μl of infection media to twice the final drug concentration (200 μM).

### Remdesivir (Positive Control) Preparation

The Remdesivir control was diluted to 40 M (twice the final concentration) by adding 3 μl of a 10 mM stock to 747 μl of supplemented media.

### Cell Treatment

Media was removed from both antiviral and cytotoxicity plates and immediately replaced with 50 μl of infection media (without diluent) followed by 50 μl of diluted compounds.

The antiviral plates were incubated for 1 hour at 37° C., 5% CO₂. The cytotoxicity plates were incubated for 24 hours at 37° C., 5% CO₂.

### Virus Preparation

The virus stock was diluted 10-fold to bring the concentration to 0.2×10⁶ TCID50/ml. 56 μl of diluted virus was transferred into 17.5 ml of supplemented media, to reach an MOI 0.002.

### Cell Infection

After 60 minutes, media was removed from the cells and replaced with 50 μl of virus (or media for the uninfected controls) and 50 μl of diluted compounds for 24 hours.

### Fixation and Development

After 24 hours, the infection plates were washed with PBS, fixed for 30 minutes with 4% formaldehyde, washed again with PBS, and stored in PBS at 4° C. until staining. The cytotoxicity plate was treated with MTT to determine cell viability.

### Infectivity Readout

Residual formaldehyde was quenched with 50 mM Ammonium Chloride, after which cells were permeabilized (0.1% Triton X 100) and stained with an antibody recognizing SARS-CoV2 Nucleocapsid protein (Thermo Fisher MA536271). The primary antibody was detected with an Alexa-488 conjugate secondary antibody (Life Technologies, A21207), and nuclei were stained with Hoechst. Images were acquired on a Cell-Insight CX5 high content platform (Thermo Scientific) using a 4× objective, and percentage infection calculated using CellInsight CX5 software (infected cells/total cells×100).

### Cytotoxicity Readout

Cytotoxicity was detected by MTT assay. Briefly, the MTT reagent (Sigma, M5655) was added to the cells for 2 hours at 37° C., 5% CO₂, after which the media was removed, and the precipitate solubilized with a mixture of 1:1 Isopropanol: DMSO for 20 minutes. The supernatant was transferred to a clean plate and signal was read at 570 nm.

### Determination of ECSO Concentration—IF Assay

Normalized percentages of inhibition were calculated using the following formula:

$$Normalized \% inhibition = 100 \times ( 1 - \frac{\% Infection Sample - \% Infection Unifected Control}{\% Infection Infected Control - \% Infection Unifected Control} )$$

EC₅₀ values were extrapolated from the curves representing the best fit (non-linear regression analysis, variable slope) of the logarithm of compound concentration vs. the normalized percentages of inhibition, using GraphPad Prism (version 9).

### Determination of TCSO Concentration

Percentages of cytotoxicity were calculated using the following formula:

$$\% Cytotoxicity = 100 \times ( 100 \times \frac{Absorbance Sample}{Absorbance Untreated Control} )$$

TC₅₀ values were extrapolated from the curves representing the best fit (non-linear regression analysis, variable slope) of the logarithm of compound concentration vs. the normalized percentages of cytotoxicity, using GraphPad Prism (version 9).

### Results

The cytotoxicity and antiviral activity of M13 was determined by adding M13 one hour prior to in vitro infection with SARS-CoV-2 of Vero cells.

Please delete table 4! It contains distracting data.

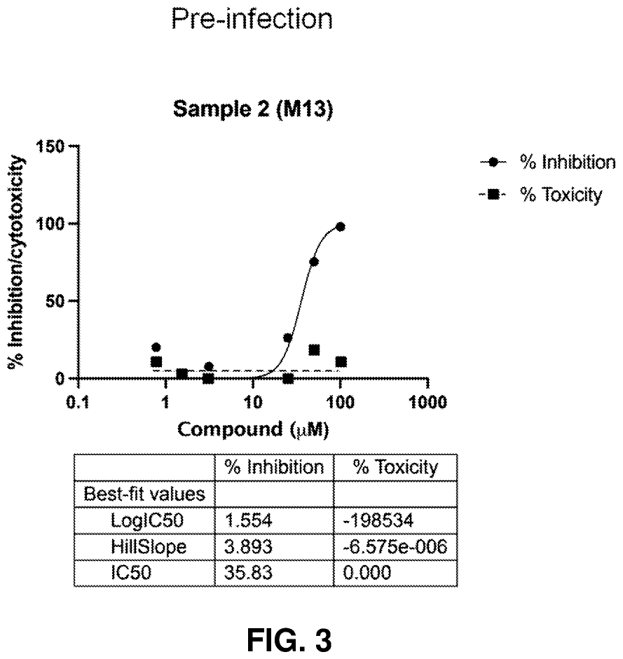

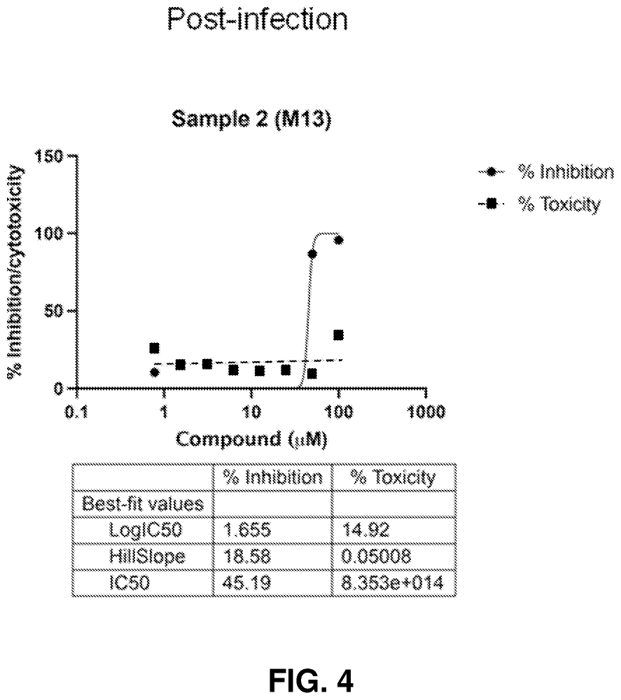

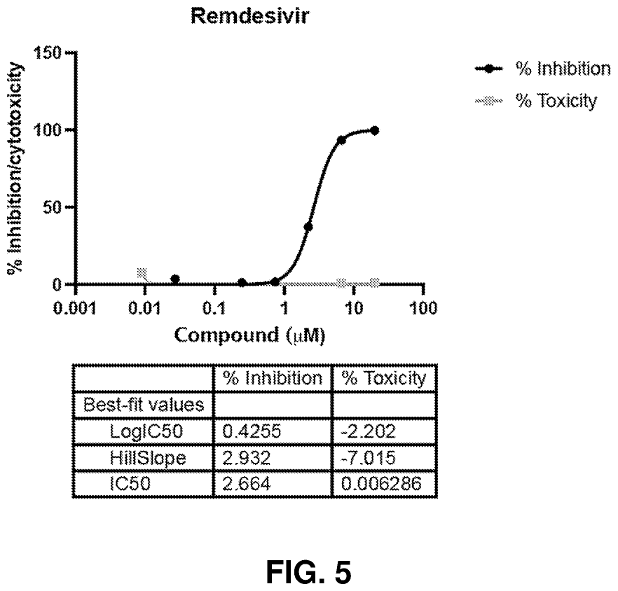

It was found that pre-incubation of Vero cells with M13 for 60 minutes, resulted in increased antiviral activity with EC50 ~36 μM against SARS-CoV-2 without significant cytotoxicity (FIG. 3). The cytotoxicity and antiviral activity of M13 was determined, when M13 was added after the in vitro infection (using Vero cells) against SARS-CoV-2. It was observed that M13, when added one-hour post-infection increased antiviral activity at levels of EC50-45 μM with low toxicity (FIG. 4). In general, under the conditions tested, M13 displayed antiviral activity against SARS-CoV-2 when pre-incubated with the cells 60 minutes prior to infection, with EC₅₀ of 35.83 μM. No significant cytotoxicity was observed at the concentrations tested.

### Example 4: Synthesis of M13 Derivatives

39 derivatives of M13 having the structure of Formula A were synthesized and 19 compounds were tested for anti-inflammatory and anti-SARS-COV-2 activities. The structures of the 39 M13 analogues are provided in Table 1.

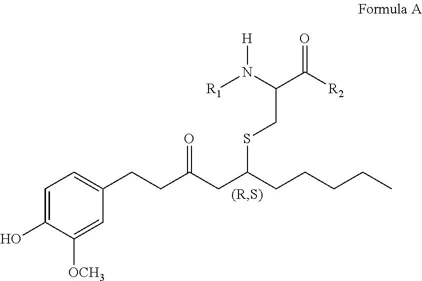

TABLE 1
Structures of the 39 M13 Analogs

| Name of | | | |
|---|---|---|---|
| Derivative | R1 | R2 | Structure |
| MLY-1 | Glu | Ala | Glu(alpha)-Cys(6-shogaol)-Ala |
| MLY-2 | Glu | Leu | Glu(alpha)-Cys(6-shogaol)-Leu |
| MLY-3 | Glu | Pro | Glu(alpha)-Cys(6-shogaol)-Pro |
| MLY-4 | Glu | Trp | Glu(alpha)-Cys(6-shogaol)-Trp |
| MLY-5 | Glu | Tyr | Glu(alpha)-Cys(6-shogaol)-Tyr |
| MLY-6 | Asp | Phe | Asp(alpha)-Cys(6-shogaol)-Phe |
| MLY-7 | Asp | Leu | Asp(alpha)-Cys(6-shogaol)-Leu |
| MLY-8 | Asp | Met | Asp(alpha)-Cys(6-shogaol)-Met |
| MLY-9 | Asp | Pro | Asp(alpha)-Cys(6-shogaol)-Pro |
| MLY-10 | Asp | Val | Asp(alpha)-Cys(6-shogaol)-Val |
| MLY-11 | Asp | Trp | Asp(alpha)-Cys(6-shogaol)-Trp |
| MLY-12 | Asp | Tyr | Asp(alpha)-Cys(6-shogaol)-Tyr |
| MLY-13 | Glu | Met | Glu(alpha)-Cys(6-shogaol)-Met |
| MLY-14 | Asp | Ile | Asp(alpha)-Cys(6-shogaol)-Ile |
| MLY-15 | Glu | Phe | Glu(alpha)-Cys(6-shogaol)-Phe |
| MLY-16 | Glu | Ile | Glu(alpha)-Cys(6-shogaol)-Ile |
| MLY-17 | Glu | Val | Glu(alpha)-Cys(6-shogaol)-Val |
| MLY-18 | Asp | Gly | Asp(alpha)-Cys(6-shogaol)-Gly |
| MLY-19 | Asp | Ala | Asp(alpha)-Cys(6-shogaol)-Ala |
| MLY-20 | Glu | Ala | Glu(gamma)-Cys(6-shogaol)-Ala |
| MLY-21 | Glu | Phe | Glu(gamma)-Cys(6-shogaol)-Phe |
| MLY-22 | Glu | Ile | Glu(gamma)-Cys(6-shogaol)-Ile |
| MLY-23 | Glu | Leu | Glu(gamma)-Cys(6-shogaol)-Leu |
| MLY-24 | Glu | Met | Glu(gamma)-Cys(6-shogaol)-Met |
| MLY-25 | Glu | Pro | Glu(gamma)-Cys(6-shogaol)-Pro |
| MLY-26 | Glu | Val | Glu(gamma)-Cys(6-shogaol)-Val |
| MLY-27 | Glu | Trp | Glu(gamma)-Cys(6-shogaol)-Trp |
| MLY-28 | Glu | Tyr | Glu(gamma)-Cys(6-shogaol)-Tyr |
| MLY-29 | Asp | Gly | Asp(beta)-Cys(6-shogaol)-Gly |
| MLY-30 | Asp | Ala | Asp(beta)-Cys(6-shogaol)-Ala |
| MLY-31 | Asp | Phe | Asp(beta)-Cys(6-shogaol)-Phe |
| MLY-32 | Asp | IIe | Asp(beta)-Cys(6-shogaol)-Ile |
| MLY-33 | Asp | Leu | Asp(beta)-Cys(6-shogaol)-Leu |
| MLY-34 | Asp | Met | Asp(beta)-Cys(6-shogaol)-Met |
| MLY-35 | Asp | Pro | Asp(beta)-Cys(6-shogaol)-Pro |
| MLY-36 | Asp | Val | Asp(beta)-Cys(6-shogaol)-Val |
| MLY-37 | Asp | Trp | Asp(beta)-Cys(6-shogaol)-Trp |
| MLY-38 | Asp | Tyr | Asp(beta)-Cys(6-shogaol)-Tyr |
| MLY-39 | Glu | Gly | Glu(alpha)-Cys(6-shogaol)-Gly |

The general semisynthetic approach is described below:

Step 1: An amount of Fmoc-Phe-Wang Resin was added to a reactor, Dimethylformamide (DMF) solvent was added, and the mixture was allowed to soak for 2 hours. The DMF was then drained off, followed by the addition of 20% Piperidine in DMF (20% Pip/DMF). The mixture was bubbled with nitrogen for 0.5 hour, rinsed with DMF 5 times, and a dark blue color was detected with ninhydrin.

Step 2A: An amount of Fmoc-Cys(6-shogaol)-OH and Hydroxy-benzo-triazole (HOBt) were weighed into a beaker, DMF was added, and the mixture was stirred and cooled to 0° C. N,N′-Di-isopropyl-carbo-di-imide (DIC) was added to the mixture for 0.5 hour to activate the reaction. The activated solution was added to resin solution from Step 1 under nitrogen atmosphere, and stirred for 1 hour. The reaction was complete when the solution was transparent as detected by ninhydrin, after which the DMF was drained. Fresh DMF was added and drained for washing three times. 20% Pip/DMF was added, the mixture was bubbled with nitrogen for 0.5 hour, and washed with DMF 5 times until a dark blue color was detected using ninhydrin.

Step 2B: An amount of Fmoc-Glu(OtBu)-OH and HOBt were weighed into a beaker, DMF was added, the mixture was stirred and cooled to 0° C. DIC was added to the mixture for 0.5 hour to activate the reaction. The activated solution was added to the resin solution from Step 1 under nitrogen atmosphere, and stirred for 1 hour. The reaction was complete when the solution turned transparent as detected with ninhydrin, after which the DMF was drained. Fresh DMF was added and drained three times for washing three. 20% Pip/DMF was added, the mixture was bubbled with nitrogen for 0.5 hour, and washed with DMF 5 times until a dark blue color was detected with ninhydrin.

Step 3: The cutting liquid containing 90% Trifluoroacetic acid (TFA), 5% Ethane-1,2-dithiol (EDT), 2.5% Titanium(JI) sulfide (TiS), and 2.5% water was prepared in a jar. The crude resin was added and the mixture was stirred at 40-45° C. for 2.5 hours, then filtered. The filtrate solution was added to methyl tert-butyl-ether, and the solid was precipitated and filtered. The crude product was washed twice with methyl tert-butyl-ether, and placed in a vacuum drying oven to dry overnight to obtain the dried crude product.

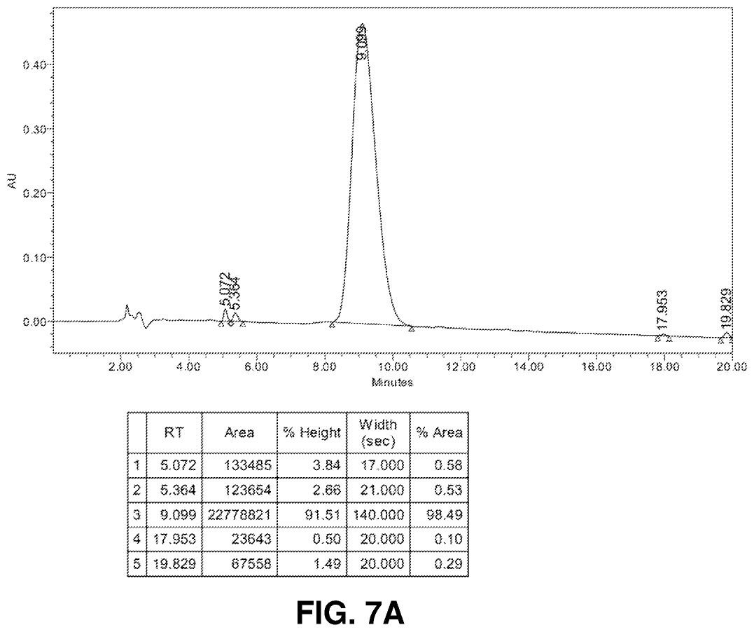

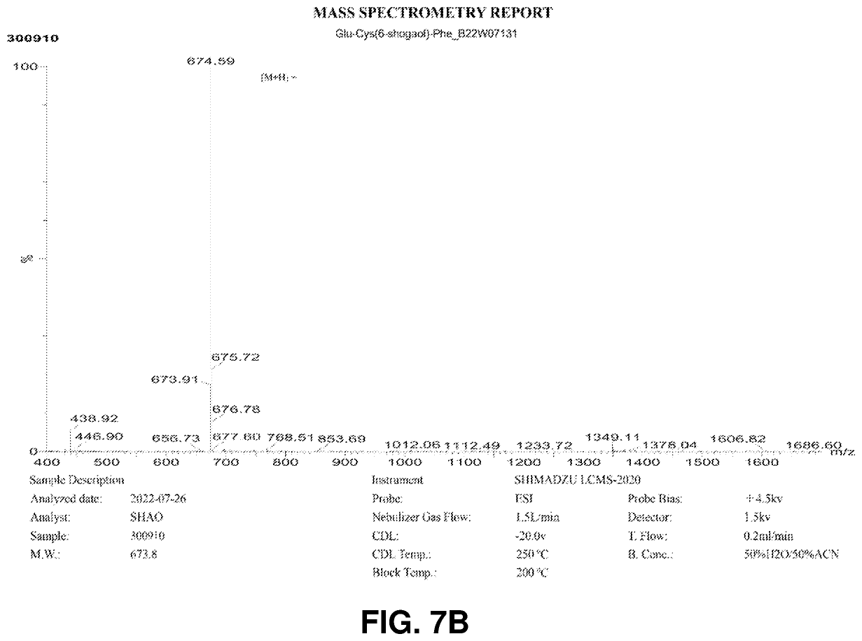

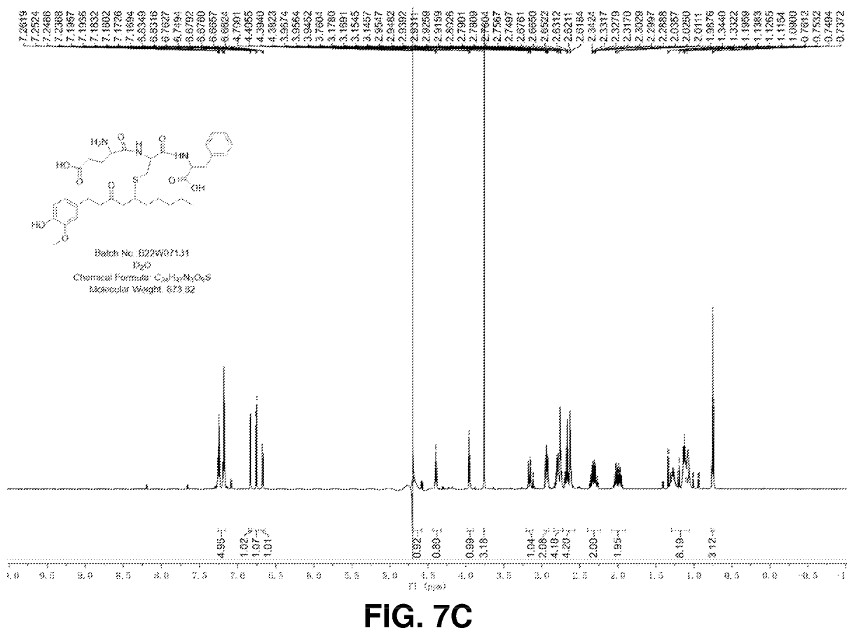

Step 4: The crude product from Step 3 was purified by HPLC chromatography and lyophilized to obtain the final product with a purity >95%. A representative chemical reaction scheme for compound MLY2 is shown in FIG. 6. FIGS. 7A-7C show a representative HPLC readout (FIG. 7A), Mass spectrum (FIG. 7B), and 1H-NMR spectrum (FIG. 7C) for the exemplary compound MLY-2.

### Example 5: Anti-Inflammatory and Antiviral Effects of the Compounds MLY1-MLY19

### Anti-inflammatory Effects of Compounds

Nuclear factor kappa B (NF-κB) is an ancient protein transcription factor (Salminen, A., et al., Bioessays 2008, 30: 939-942) and is considered a regulator of innate immunity (Baltimore D. Discovering NF-kappaB. Cold Spring Harb Perspect Biol. 2009, 1(1): a000026). The NF-κB signaling pathway links pathogenic signals and cellular danger signals, thus organizing cellular resistance to invading pathogens. In the current study, the anti-inflammatory activities of the compounds MLY-1-MLY-19 were compared with that of M13.

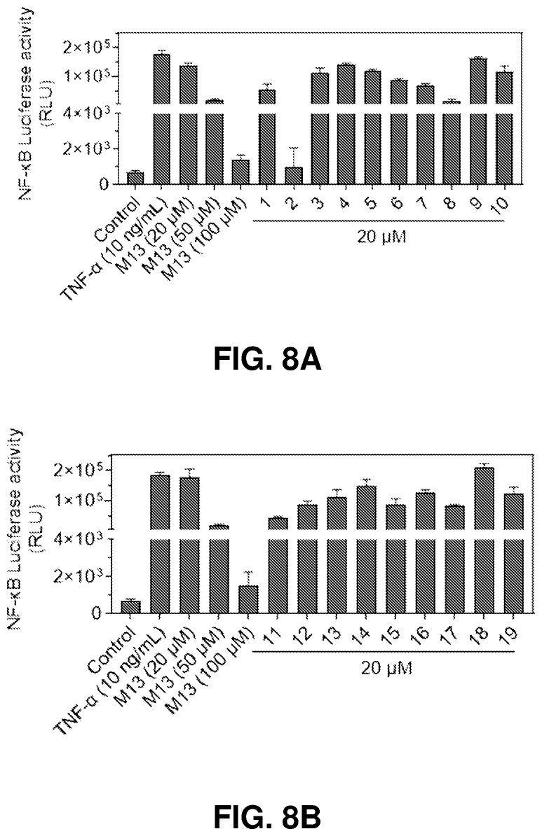

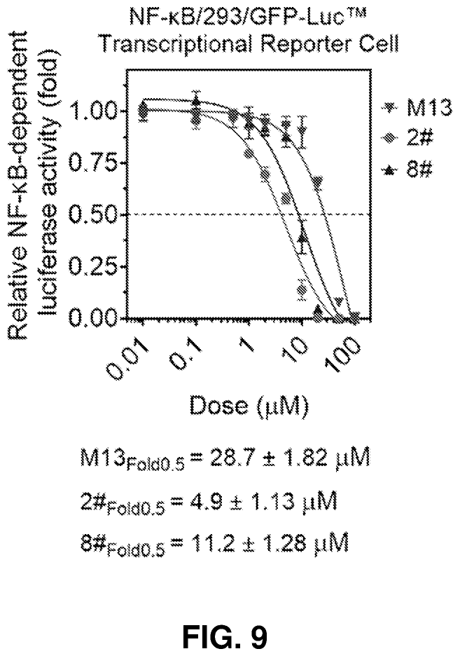

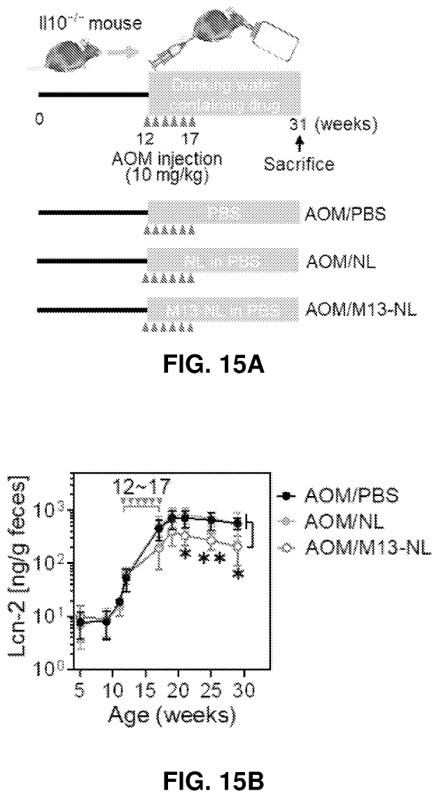

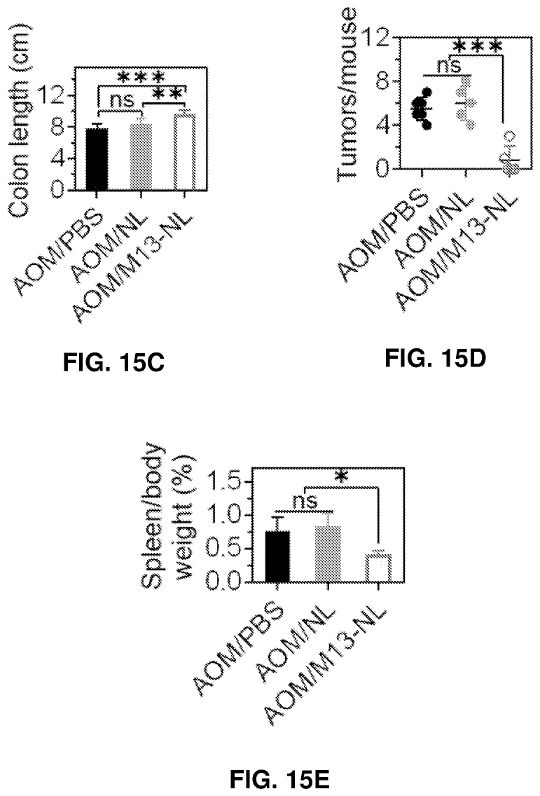

First, the anti-inflammatory activity of MLY compounds on the NF-κB signaling pathway was examined using the NF-xB/293/GFP-LucTnl cell line (System Biosciences). This cell line is specifically designed for use in quantitatively monitoring (by luciferase intensity) transcriptional activation related to the NF-κB signal transduction pathway in vitro. Treatment of NF-κB/293/GFP-Luc™ cells with 10 ng/ml TNF-α for 18 hours induced strong activation of the NF-κB signal transduction pathway (FIGS. 8A and 8B). This activation was reduced in the presence of 20 M of the 17 MLY compounds (FIGS. 8A and 8B) and 15 MLY compounds have an anti-inflammatory activity superior to the anti-inflammatory activity of M13 (FIGS. 8A and 8B, Tables 2A and 2B). MLY2 and MLY8 induced a strongest 90% reduction of NF-κB activation the anti-inflammatory effect of MLY 2 and 8 was dose-related (FIG. 9). The dose to reduce the NF-κB activation by 50% was ~29 M for M13, 5 μM for MLY2 and 11 μM for MLY8 (FIG. 9).

TABLE 2A
Anti-inflammatory Activity of the Derivative Compounds

| | RLU | Order of |
|---|---|---|
| | (% with TNFα) | Activity |
| Control | 0.39 ± 0.06 | |
| TNF-α (10 ng/ml) | 100 ± 7.66 | |
| M13 (20 μM) | 87.25 ± 8.29 | |
| M13 (50 μM) | 9.75 ± 2.78 | |
| M13 (100 μM) | 0.81 ± 0.22 | |
| MLY-1 | 30.3 ± 11.78 | |
| MLY-2 | 0.78 ± 0.54 | (1) |
| MLY-3 | 6.54 ± 8.56 | (11) |
| MLY-4 | 80.65 ± 2.22 | (16) |
| MLY-5 | 67.18 ± 3.27 | (14) |
| MLY-6 | 50.7 ± 1.38 | (9) |
| MLY-7 | 38.6 ± 3.96 | (5) |
| MLY-8 | 8.04 ± 4.06 | (2) |
| MLY-9 | 92.85 ± 2.15 | (18) |
| MLY-10 | 66.68 ± 11.06 | (12) |
| MLY-11 | 24.75 ± 0.79 | (3) |
| MLY-12 | 47.43 ± 5.5 | (7) |
| MLY-13 | 60.22 ± 13.55 | (10) |
| MLY-14 | 80.67 ± 11.28 | (17) |
| MLY-15 | 47.98 ± 9.35 | (8) |
| MLY-16 | 68.19 ± 4.46 | (15) |
| MLY-17 | 45.13 ±1.96 | (6) |
| MLY-18 | 112.31 ± 7.28 | (19) |
| MLY-19 | 67.04 ± 11.04 | (13) |

TABLE 2B
Anti-inflammatory Activity of the M13 Analogs

| RLU | | Activity compared |
|---|---|---|
| (% with TNF-α) | Compound Number | with M13 |
| 0~5% | 2 | >>>>M13 |
| 5%~15% | 8 | >>>M13 |
| 15~35% | 11, 1 | >>M13 |
| 35%~70% | 7, 17, 12, 15, 6, | >M13 |
| | 13, 3, 10, 19, 5, 16 | |
| 70%~85% | 4, 14 | = M13 |
| >90% | 9, 18 | <M13 |

The expression of cytokines in inflamed macrophages (Raw 264.7 cells stimulated by LPS) was measured in the presence or in absence of M13 and the MLY compounds using a Proteome Profiler^{T}mi Human XL Oncology Array kit (R&D Systems, Cat #ARY026, Abingdon, United Kingdom) according to manufacturer's instructions. Briefly, cell lysates (200 μg) were incubated with each array overnight at 4° C. on a rocking platform shaker. After removing the cell lysates and washing the membranes 3 times with wash buffer, the arrays were incubated with a detection antibody cocktail for 1 hour at room temperature. Then, 2 ml of Streptavidin-HRP solution was added to each membrane for a 30-minute incubation, followed by three washes. The labeled protein spots were visualized using Chemi Reagent Mix for a 1-minute incubation, and detected using the ChemiDoc MP imaging system (Bio-Rad) and Image Lab software (version 4.1, Bio-Rad). The intensities of the resulting spots were calculated with Image Studiom™ Lite software (Version 5.2), and correspond to the mean±standard deviation of four independent experiments.

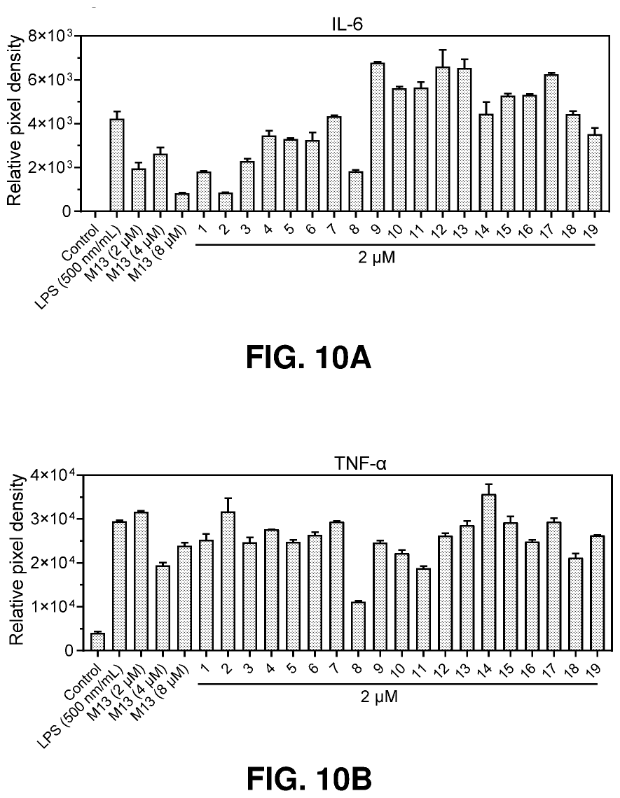

The MLY compounds decreased IL-6 and TNF-α expression in vitro. For example, although cells treated with 7 MLY compounds had decreased expression of IL-6 compared to LPS-treated cells, with similar potency as M13 (FIG. 10A). It was also observed that 13 out of 19 MLY compounds reduced TNFα expression relative to LPS-treated cells (FIG. 10B).

### Antiviral Effects of Compounds

The antiviral activity of selected compounds against SARS-CoV-2 was tested in Vero cells. Vero cells were infected with SARS-CoV-2 at a multiplicity of infection (MOI) of 1 for 1 hour, followed by the addition of candidate MLY compounds. Cell pellets were collected at 24 hours after infection. Virus RNA copies were measured by RT-PCR. The strain of coronavirus assayed was the SARS-COV-2 (USA-WA1/2020) Wuhan strain (BEI NR-52281). The viral strain was amplified in Vero cells and had titers of 5×10⁶ plaque-forming units (PFU)/mL. Vero cells were grown in DMEM (Gibco) supplemented with 5% heat-inactivated fetal bovine serum. Virus RNA levels were analyzed by quantitative reverse transcription-polymerase chain reaction (qRT-PCR). RNA from cell pellets was extracted using a Viral RNA Mini Kit (Qiagen). qRT-PCR was used to measure viral RNA levels using the following primers and probes specific for the SARS-COV-2 virus N1 (Integrated DNA Technologies):

Forward primer:
(SEQ ID NO: 1)
5′-CCGCTGCCCAACACAAG-3′,
Reverse primer:
(SEQ ID NO: 2)
5′-CCACTAACGTTCTTTTGCAGACAT-3′,
Probe,
(SEQ ID NO: 3)
5′-/56-FAM/AGCCTACCT/ZEN/TGACAAGCAATCAGACACTCAA/
3IABkFQ/-3′

Viral RNA copies were determined after comparison with a standard curve produced using serial 10-fold dilutions of SARS-CoV-2 RNA. Two independent experiments were conducted in duplicates. The results are shown in Table 8 below. The results demonstrate that administration of 17 out of 19 MLY compounds reduced the SARS-CoV-2 infection and MLY and MLY 4, 5, 7, 8, 11, 12, 17 and 19 reduce SARS-CoV-2 by 92.0% to 99.9% post-infection.

TABLE 3
Post SARS-CoV-2 Infection Results of MLY 1-19 Compounds

| Sample | | | | | | | Percent |
|---|---|---|---|---|---|---|---|
| Name | Target | CT | | Average | Log | Percent | Reduction |
| M13 | N1 | 21.163 | 20.673 | 20.918 | 1.0E+04 | 0.09 | 99.91 |
| MLY-1 | N1 | 9.151 | 9.343 | 9.247 | 9.2E+06 | 76.63 | 23.37 |
| MLY-2 | N1 | 9.508 | 9.399 | 9.454 | 8.2E+06 | 67.95 | 32.05 |
| MLY-3 | N1 | 9.007 | 9.161 | 9.084 | 1.0E+07 | 84.29 | 15.71 |
| MLY-4 | N1 | 13.159 | 13.608 | 13.384 | 8.3E+05 | 6.90 | 93.10 |
| MLY-5 | N1 | 14.952 | 15.433 | 15.193 | 2.9E+05 | 2.41 | 97.59 |
| MLY-6 | N1 | 12.073 | 10.964 | 11.518 | 2.5E+06 | 20.43 | 79.57 |
| MLY-7 | N1 | 15.923 | 16.145 | 16.034 | 1.8E+05 | 1.48 | 98.52 |
| MLY-8 | N1 | 15.886 | 14.804 | 15.345 | 2.6E+05 | 2.20 | 97.80 |
| MLY-9 | N1 | 9.302 | 9.291 | 9.296 | 8.9E+06 | 74.47 | 25.53 |
| MLY-10 | N1 | 11.367 | 10.755 | 11.061 | 3.2E+06 | 26.67 | 73.33 |
| MLY-11 | N1 | 16.024 | 15.789 | 15.906 | 1.9E+05 | 1.59 | 98.41 |
| MLY-12 | N1 | 13.575 | 12.684 | 13.130 | 9.6E+05 | 8.00 | 92.00 |
| MLY-13 | N1 | 12.117 | 12.711 | 12.414 | 1.5E+06 | 12.14 | 87.86 |
| MLY-16 | N1 | 9.340 | 9.267 | 9.304 | 8.9E+06 | 74.15 | 25.85 |
| MLY-17 | N1 | 22.076 | 24.144 | 23.110 | 2.9E+03 | 0.02 | 99.98 |
| MLY-18 | NI | 12.302 | 12.952 | 12.627 | 1.3E+06 | 10.72 | 89.28 |
| MLY-19 | N1 | 14.510 | 15.082 | 14.796 | 3.6E+05 | 3.03 | 96.97 |
| Media Inf | N1 | 8.677 | 8.903 | 8.790 | 1.2E+07 | 100.00 | 0.00 |
| Media CON | N1 | 36.474 | 33.806 | 35.140 | 2.6E+00 | 0.00 | 100.00 |

These results indicate that the MLY compounds are potent candidates to ameliorate the development of COVID-19, by a combined mechanism entailing downregulation of excessive inflammatory reactions, increased cell protection, and increased antiviral effects.

### Example 6: Oral Administration of M13-Loaded Lipid Nanoparticles is Safe and Effective for Treating Colitis-Associated Cancer

### Materials and Methods

The chemical synthesis of M13 was carried out as previously described by Zhu et al. (2013)[14], with some modifications.

### Assembly of M13-Loaded Nanoliposomes

To fabricate M13-NL, lipids were extracted from GDNPs using a modified liquid-liquid extraction method and loaded them with M13 to form M13-NL. The formed M13-NLs were spherical with an average size of 220 nm and a zeta potential of −16.8 millivolts. Nanoliposomes (NL) and the lipophilic carbocyanine dye (DiL)-labeled NL (DiL-NL) were also prepared. The morphologies, particle sizes, and surface charges of NL and DiL-NL were consistent with that of M13-NL.

### Cell Cultures

PBMCs were provided by STEMCELL Technologies (STEMCELL Canada Inc.) and maintained in ImmunoCult™-XF T-cell Expansion Medium (Cat #10981). HIEC-6 cells were maintained in OptiMEM 1 Reduced Serum Medium supplemented with 20 mM HEPES, 10 mM GlutaMAX, 10 ng/mL Epidermal Growth Factor, 4% (v/v) fetal bovine serum (FBS), and 1% penicillin-streptomycin (PS). HCT-116 cells were maintained in ATCC-formulated McCoy's 5a medium, supplemented with 10% (v/v) FBS and 1% PS. Caco-2/BBe cells were grown in DMEM supplemented with 10% (v/v) FBS and 1% PS (v/v). Human colon cancer stem cells (HC-CSC) were maintained in complete growth media with serum and antibiotics (Cat #M36112-39S) from Celprogen Inc. (Torrance, CA, USA).

### Animals

Female C57BL/6J mice (Strain #:000664, The Jackson Laboratory, Bar Harbor, ME), female interleukin-10 knock-out (IL10^{-/-} mice, B6.129P2-IL10 tmlCgn, The Jackson Laboratory), and female CD-1 mice (CD-1@IGS Mouse, outbred, Charles River) were housed in a clean facility maintained at 22±2° C. and 30-70% relative humidity with a 12-h light/dark cycle. The diet (ABDIET@PICOLAB®, PMI Nutrition International LLC, Arden Hills, MN, USA) and water were provided ad libitum. Mice experiments were performed following ARRIVE guidelines 2.0, with approval from the Georgia State University Institutional Animal Care and Use Committee (IACUC, Protocol #A₂₀₀₃₉).

### Statistics

Data represent mean values ±standard deviation (SD) from at least 3 independent experiments. One-way ANOVA was used to determine statistical significance. All statistical analyses were performed using GraphPad Prism 8.0 (GraphPad Software Inc.). Statistical significance was set at P<0.05.

### Results

### M13 has Excellent Biopharmaceutical Properties and Biosafety

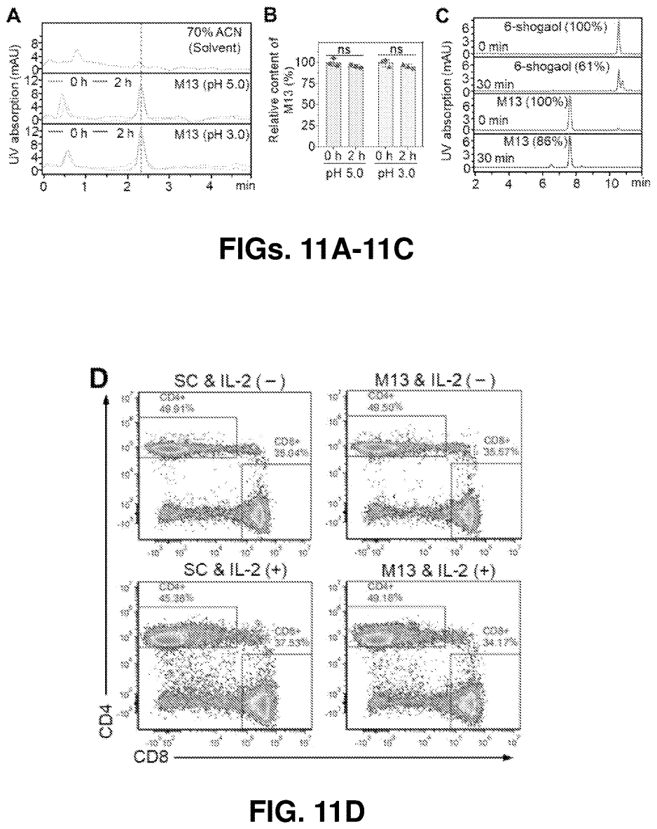

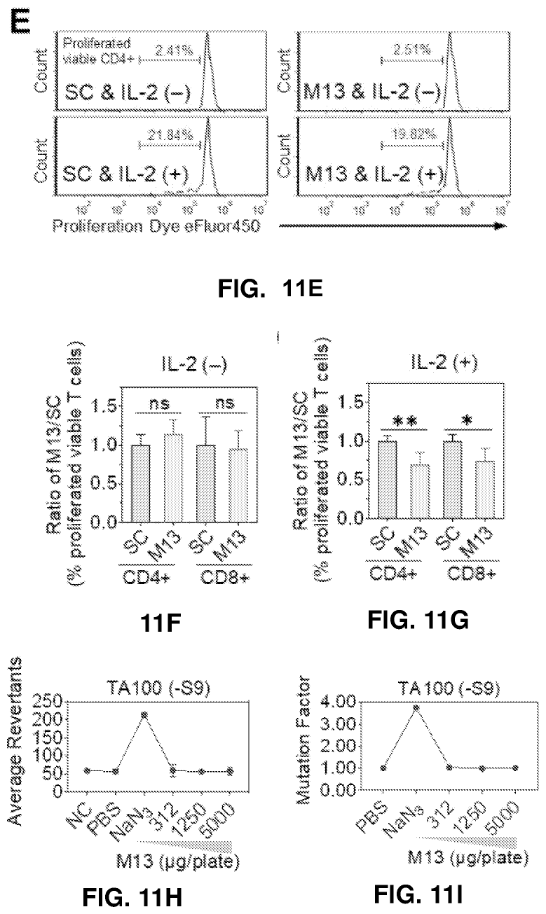

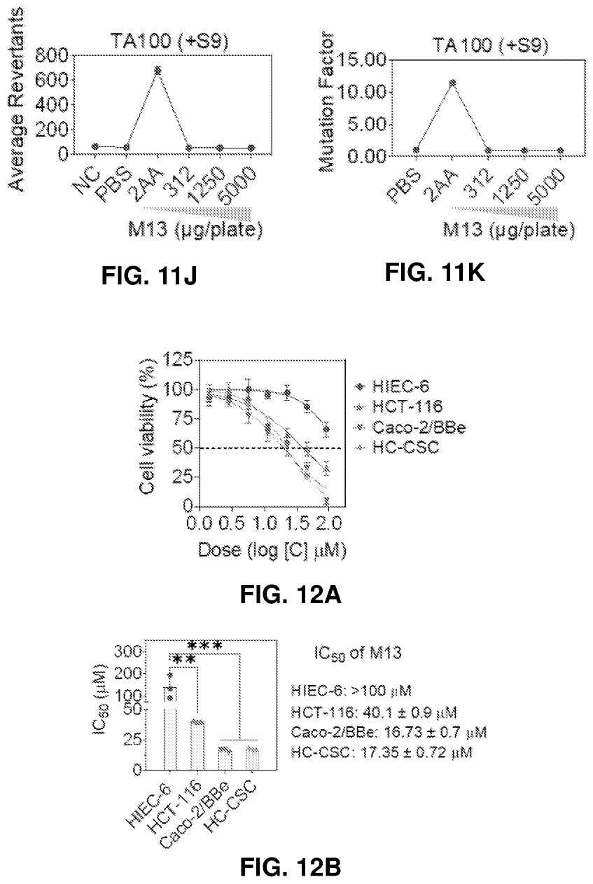

The biopharmaceutical properties of the synthesized M13 (5-glutathione-6-shogaol) were characterized. HPLC analysis showed that the relative content of M13 was consistent before and after incubation at 37° C. for 2 h in acidic PBS (pH=5.0 or 3.0) (FIGS. 11A and 11B), indicating that M13 is chemically stable at acidic pH. After incubation for 30 minutes with mouse intestinal microsomes, the M13 sample retained 86% of the input, whereas 6-shogaol retained only 61% of the input (FIG. 11C), indicating that M13 exhibits a lower metabolic liability than 6-shogaol. M13 also showed high aqueous solubility, as determined by its kinetic solubility (>200 μM) and thermodynamic (or equilibrium) solubility (1390.03 μM), and the partition of M13 between the organic solvent (octanol) and aqueous buffer (characterized with a Log D7.4 of −1.75). The ionization/protonation constants (pKa) were M13 pKa1=3.24, M13 pKa2=9.12, and M13 pKa3=10.42, as determined by UV metric at pH 2-12. This indicates that M13 is only partially ionized at physiological pH (6-7), suggesting that it has a high permeability across cell membranes.

To evaluate the immunogenic effects of M13 on human T lymphocytes, PBMCs were cultured in blank medium (standard control), medium containing CD3/CD28 activator (positive control), medium containing solvent (solvent control, SC), and medium containing M13 solution (20 μM). All the groups were cultured in the presence or absence of interleukin-2 (IL-2). After a 4-day incubation, PBMCs were labeled with immunofluorescent dyes and analyzed using flow cytometry. The results showed that, in resting-stage T lymphocytes (without IL-2 supplementation), the percentages of CD4⁺ and CD8⁺ T-cell populations and the T-cell proliferation status were similar in the M13-treated, SC, and standard control groups (FIGS. 11D-11F). This indicated that M13 treatment is safe for resting lymphocytes. Interestingly, in activated-stage T lymphocytes (with IL-2 supplementation), the M13-treated group exhibited reduced proliferation of activated CD4⁺ and CD8⁺ lymphocytes compared to the SC and standard control groups (FIGS. 11D, 11E, and 11G). This suggests that M13 may suppress CD4⁺ and CD8⁺ cells in IBD patients with activated lymphocytes in a manner similar to the action of anti-TNF-α drugs (e.g., infliximab) [Dahlen, et al., Scand J Immunol 2013, 78 (3), 275].

Next, the mutagenic potential of M13 using a Salmonella typhimurium reverse mutation assay were assessed (Ames test). Different concentrations of M13 (156-5000 μg/plate), solvent (PBS), positive controls (NaN3, 2AA, or 2NF), and negative (untreated) controls were tested on two S. typhimurium tester strains (TA98 and TA100) with (+S9) or without (−S9) metabolic activation. It was found that M13 did not increase the number of revertant colonies at any tested concentration, and the mutation factors were less than 1.6 for all concentrations of M13 in strains TA98 and TA100 (FIGS. 11H-11K). These results indicated that M13 was not mutagenic.

Safety screening of M13 was performed using an in vitro evaluation of safety and toxicity (InVEST). The effects of M13 was tested against 25 selected targets, including factors known to be involved in toxicity in humans. The results showed that M13 did not inhibit G-protein-coupled receptor (GPCR) targets, an ion channel (5-HT3), a phosphodiesterase, or the protease thrombin alpha, and did not significantly modulate the activity of the nuclear receptor, AHR. Collectively, the results indicate that M13 exhibits parameters consistent with its excellent safety in vitro.

To further determine the potential acute toxicity of M13 in vivo, a single-dose maximum tolerated dose (MTD) study of M13 was performed in CD-1 mice. Eight-week-old female mice were orally administered a single dose of M13 (1000 mg/kg) or PBS (as control) on day 0 and subjected to regular water and diet for 7 days. During the experiment, no significant death or differences in the physiological indices of mice between the M13 and control groups were observed. There was no significant difference in the body weight change, spleen-to-body weight ratio, or blood physiological and biochemical indices between the two groups of mice on day 7, and no pathological signs were observed in the anatomical organs. These results demonstrate that oral administration of 1000 mg/kg M13 is safe for CD-1 mice.

### M13 Inhibits the Growth of 2D- and 3D-Cultured Cancerous Intestinal Cells

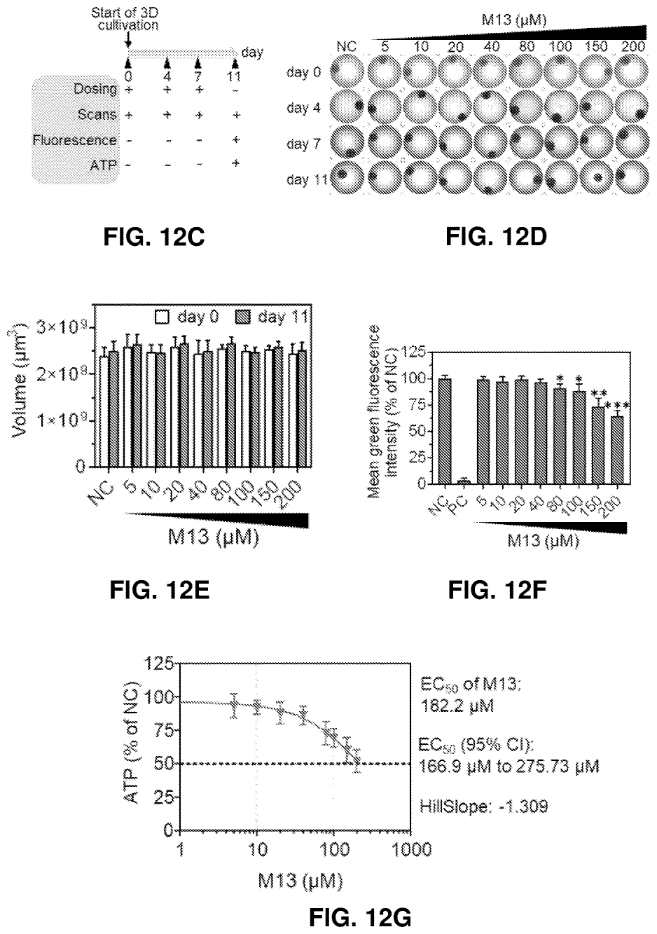

The 2,5-diphenyl-2H-tetrazolium bromide (MTT) assay was used to study the effects of M13 on the viability of four 2D-cultured human intestinal cell lines (HIEC-6, HCT-116, Caco-2/BBe, and HC-CSC). It was found that M13 at concentrations ranging from 0.1 to 100 M dose-dependently decreased the viability of all four intestinal cell lines (FIG. 12A). Importantly, M13 showed much stronger cytotoxicity toward the cancerous intestinal cell lines (HCT-116, Caco-2/BBe, and HC-CSC) than to the normal intestinal cell line (HIEC-6) (FIG. 12A). As shown in FIG. 12B, the IC50 value of M13 against HIEC-6 cells (>100 M) was significantly higher (by 2.5-, 6-, and 5.8-fold, respectively) than that of the cancerous HCT-116 (40.1 μM), Caco-2/BBe (16.73 M), and HC-CSC (17.35 μM) cells. These results demonstrated that M13 preferentially decreased the viability of cancerous intestinal cells compared to that of normal intestinal cells.

To assess the long-term cytotoxicity of M13 against cancer cells, a 3D droplet-based Caco-2/BBe cell spheroid model was established using 3D printing. The diameter of the 3D cell spheres was approximately 1.65-1.75 mm. The 3D-cultured cells were incubated with different concentrations of M13 for 11 days, and droplet size (calculated using scanned images) and viability (live/dead cell staining and intracellular ATP) were analyzed (FIG. 12C). It was observed that as the culture time increased, the color of the 3D cell spheres gradually deepened (FIG. 12D); however, their sizes did not change significantly (FIG. 12E), regardless of the group. Live/dead cell staining showed that the number of dead cells (red fluorescence under propidium iodide staining) in the 3D cell spheres was positively correlated with M13 concentration, and the number of living cells (green fluorescence under acridine orange staining) decreased as the M13 concentration increased. The cell viability of the M13-treated spheres was normalized as a percentage of the negative control (NC, 100% viable, without M13 treatment). This calculation revealed that the green fluorescence intensity of 3D cell spheres treated with M13 at a concentration >80 μM was significantly lower than that of the NC group (FIG. 12F). The ATP content in 3D- cultured Caco-2/BBe cells was quantified and it was found that the half-maximal effective concentration (EC50) of M13 was 182.2 μM with a 95% confidence interval (95% CI) (FIG. 12G). Together, these results show that Caco-2/BBe cells cultured in 3D spheres are more resistant to M13 than the same cells grown in 2D monolayers, but M13 still clearly exerts a long-term inhibitory effect against 3D-cultured cancerous intestinal cells.

### M13-NL is Internalized by Cancerous Epithelial Cells and Down-Regulates Cancer-Related Proteins and Cell Cycle Proteins In Vitro

Caco-2/BBe cells were used to evaluate the NL cellular uptake in vitro. Compared with control cells incubated without DiL-labeled NL (DiL-NL), cells incubated with DiL-NL exhibited obvious red fluorescence signals after 24 hours. In the latter group, the red fluorescence intensity of DiL-NL significantly increased with the incubation time (12, 24, and 48 hours), indicating that an increasing amount of DiL-NL was internalized by Caco-2/BBe cells. The cellular uptake efficiency of DiL-NL was quantified using a flow cytometer, which confirmed that the cells exhibited higher NL uptake as the incubation time was extended to 48 hours (data not shown). The above results indicate that NL could be continuously and efficiently taken up by and accumulated within Caco-2/BBe cells for at least 48 hours.

Next, the anti-cancer activities of free M13, NL, and M13-NL were compared against Caco-2/BBe cells. After a 24-h incubation, M13-NL showed stronger cytotoxicity than free M13, likely reflecting the enhanced uptake of M13-NL by Caco-2/BBe cells (FIG. 16A). The IC50 value of M13-NL against Caco-2/BBe cells was 8.64±1.02 RM, which was 2-fold lower than that of free M13. As expected, the NL group exhibited higher cell viability than the M13 and MI3-NL groups. There was no significant difference in the viability of cells treated with different concentrations of NL, indicating that NL is biocompatible (FIG. 16A). The above results suggest that NL is a non-toxic drug carrier that can significantly reduce the required drug dose of M13, and that M13-NL is more potent than free M13 in inhibiting the viability of cancerous intestinal cells in vitro.

The mechanism by which M13 exerts its anti-cancer effects was explored. Cell lysates of Caco-2/BBe cells incubated for 24 hours with free M13, NL, or M13-NL were hybridized on cancer-related protein array membranes (Proteome Profiler™ Human XL Oncology Array), and cell cycle control/phosphorylation profiling protein antibody microarray slides (Cell Cycle Control Phospho Antibody Array). The results from the human oncology array showed that 22 cancer-related proteins were significantly down-regulated in the free M13- or M13-NL-treated groups compared to the control group (without treatment) (Table S2). M13-NL treatment induced much greater down-regulation of cancer-related proteins than free M13 treatment. Among the 22 cancer-related proteins, survivin, EGFR, BCL-x, Dkk-1, and EpCAM were down-regulated more than 2-fold in M13-NL-treated cells (FIGS. 16B-16F). Differences in the expression levels of 238 cell cycle control/phosphorylation proteins were observed in the free M13- and/or M13-NL-treated groups compared with the control and/or NL groups: CDC25A and phosphorylated CDC25A (p-CDC25A) were down-regulated in the free M13- and M13-NL-treated groups compared to the control and NL groups (FIG. 16G). Moreover, RAD52 was down-regulated in the M13-NL-treated group compared with the other groups, but there was no significant between-group change in phosphorylated RAD52 (p-RAD52) (FIG. 16H). Since these proteins are involved in various signaling pathways and physiological reactions, it was hypothesized that M13 may significantly down-regulate the expression levels of various cancer-related proteins and cell cycle control/phosphorylation proteins to inhibit cell proliferation (CDC25A and RAD52[17]), migration (EpCAM[18]), and metastasis (Dkk-1 [19]) and promote cell apoptosis (survivin, EGFR, and BCL-x[20]) in CRC (FIG. 16I).

### Orally Administered M13-Loaded Lipid Nanoparticles (M13-NL) Yields Potent Anti-Cancer Effects in AOM/DSS-Induced CAC Mice

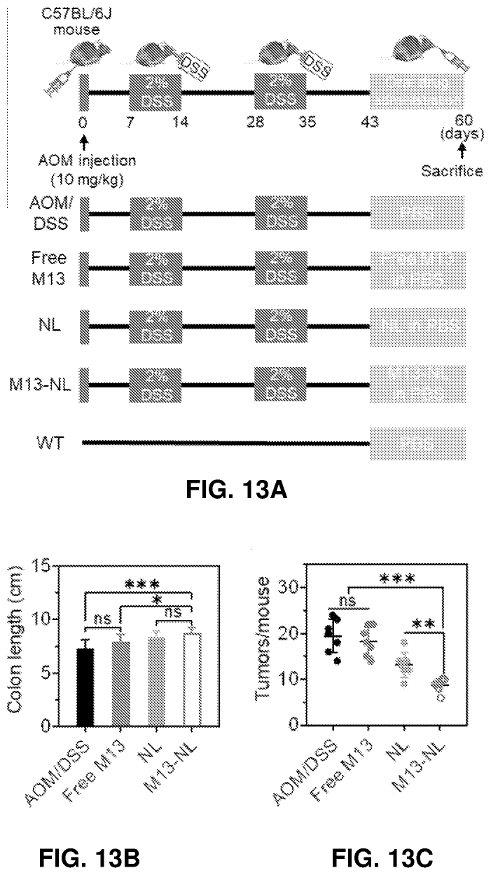

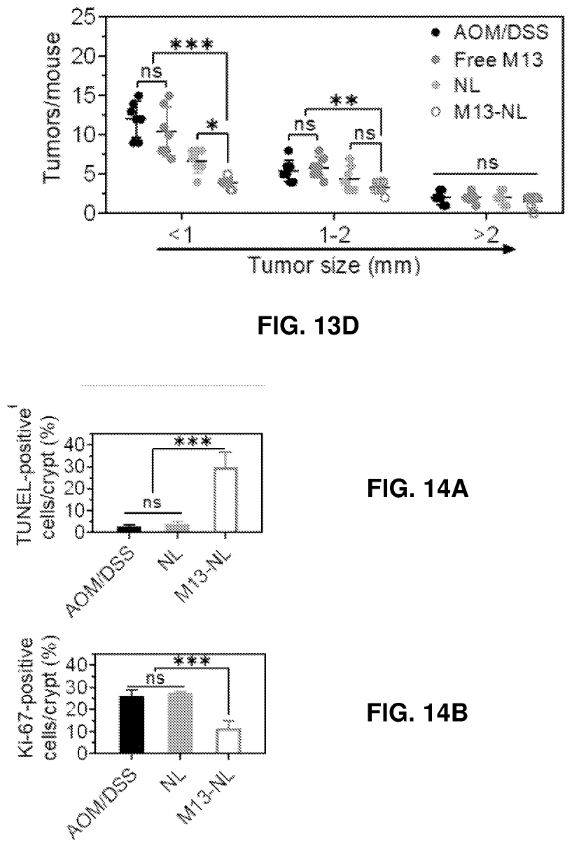

An AOM/DSS-induced CAC mouse model (C₅₇BL/6J) was used to test the anti-cancer effects of free M13, NL, and M13-NL. As shown in FIG. 13A, AOM/DSS-induced CAC mice (females, n=7 per group) were orally administered PBS (AOM/DSS group), free M13 (5 mg/kg), NL (5 mg/kg), or M13-NL (5 mg/kg of M13 loaded into 5 mg/kg of NL) via gavage every 2 days. Healthy mice were used as the control group (WT group). After sacrificing the mice at day 60, it was found that DSS induced severe inflammation in the colon, resulting in shortened colon length in mice in the AOM/DSS group. In comparison, colon length was significantly increased in the M13-NL-treated group, but not in the free M13-treated group (FIG. 13B), compared to the AOM/DSS group. When the colons of each group were dissected longitudinally, it was found that M13-NL treatment significantly reduced the number of mid-sized tumors (diameter 1-2 mm) compared with the free M13 or AOM/DSS groups, and significantly reduced the number of small-sized tumors (diameter <1 mm) compared with the NL group (FIGS. 13C and 13D). In contrast, free M13 treatment failed to reduce the number of tumors compared with that in the AOM/DSS group. These results indicate that oral M13-NL is better than free M13 and has the strongest anti-tumor activity among the tested groups in the AOM/DSS-induced CRC mouse model.

### Orally Administered M13-NL Accelerates the Recovery of Colon Tumor Tissues and Down-Regulates Cancer-Related Proteins and Cell Cycle Proteins in AOM/DSS-Induced CAC Mice

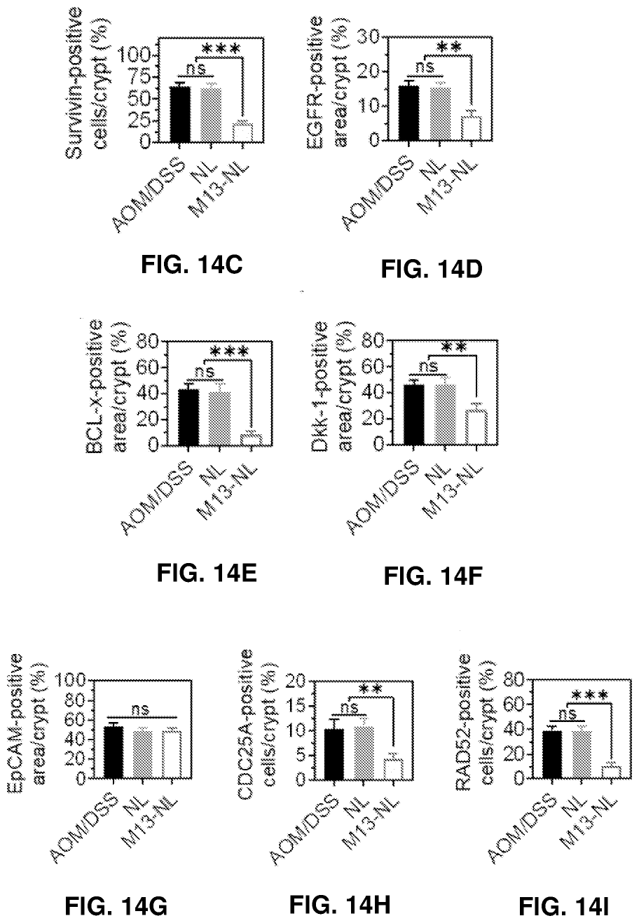

Histological assessments of the colon tissues from AOM/DSS-induced CAC mice were performed. H&E staining clearly showed hyperplasia, dysplasia, loss of epithelial cells, and expansion of cells into the lamina propria, which are typical signs of adenomas in AOM/DSS mice. In contrast, tissues from the WT mice showed normal histological structures. Colon tissues of mice in the NL and AOM/DSS groups showed signs of advanced adenomas, whereas colon tissues of mice in the M13-NL group showed signs of early adenoma and appeared less severely affected than those in the AOM/DSS group. Immunofluorescence (IF) staining of apoptotic cells (TUNEL) and immunohistochemical (IHC) staining of Ki-67 were used to investigate the pro-apoptotic and anti-proliferative effects of M13-NL in vivo. Tumor sections from the AOM/DSS group showed less green fluorescence from TUNEL-positive cells, whereas those from the M13-NL-treated group showed significantly stronger green fluorescence signals than those from the AOM/DSS group (FIG. 14A). IHC staining showed that the number of Ki-67-positive cells was significantly reduced in the M13-NL-treated group compared to that in the AOM/DSS group (FIG. 14B). Colon tissues from the WT and NL groups were analyzed using TUNEL and Ki-67 staining, and quantitative analysis showed that there was no significant difference in the percentage of TUNEL- or Ki-67-positive cells per crypt between the AOM/DSS and NL groups (FIGS. 14A and 14B).

IHC staining showed that M13-NL treatment significantly reduced the expression levels of survivin, EGFR, BCL-x, Dkk-1, CDC25A, and RAD52, but not EpCAM, in CAC colon tissues compared to those in the AOM/DSS and NL groups (FIGS. 14C-141). Collectively, these data demonstrate that oral administration of M13-NL yields potent anti-cancer effects by down-regulating various cancer-related proteins and key cell cycle proteins in the colon of CAC mice.

Moreover, changes in body weight and spleen-to-body weight ratio, H&E staining of major organs, and hematological and biochemical indicators of blood were measured as part of an initial safety evaluation. No significant changes in body weight or spleen-to-body weight ratio, no major organ lesions, and no significant changes in the hematological or biochemical parameters of the blood in the mice treated with M13-NL or empty NL. The results demonstrated that oral treatment with M13-NL delivered M13 to the colon without inducing systemic side effects in mice.

Orally administered M13-NL prevents colonic tumorigenesis in AOM-exposed IL10^{-/-} mice

Next, an AOM-exposed IL10^{-/-} mouse model was used to evaluate the preventive effects of long-term oral administration of M13-NL on colon tumorigenesis. As shown in FIG. 15A, 12-week-old IL10^{-/-} female mice were randomly allocated to three groups (n=5 or 6 per group) and orally administered PBS (AOM/PBS group), PBS with NL [0.0025% (m/v), i.e. AOM/NL group], or PBS with M13-NL [0.0025% M13 (m/v) loaded into 0.0025% NL, i.e. AOM/M13-NL group] via drinking water from the age of 12 to 31 weeks. At 12 weeks of age, the mice were given intraperitoneal injections of AOM (10 mg/kg) once a week for 6 weeks. The effect of oral PBS, NL, or M13-NL on the survival and body weight of AOM-exposed IL10^{-/-} mice was measured throughout the experiment. No mortality was observed in most of the groups during the study period. It was observed that the body weights of the mice in the three groups decreased slightly during AOM injection (12 to 17 weeks) and gradually increased thereafter until sacrifice, and there was no significant difference in the body weight changes of mice among the three groups. These results suggest that long-term M13-NL treatment does not induce severe systemic toxicity in these mice. Next, the anti-inflammatory effect of M13-NL after long-term treatment was assessed by detecting fecal lipocalin 2 (Lcn-2), a non-invasive biomarker for monitoring intestinal inflammation in mice [21]. It was observed that the levels of fecal Lcn-2 gradually increased in all groups from weeks 11 to 19 (FIG. 15B). Compared to the AOM/PBS and AOM/NL groups, the fecal Lcn-2 concentration in the AOM/M13-NL group showed a downward trend beginning at week 17 and was significantly lower than that in the AOM/PBS and AOM/NL groups from week 21 to the end of the experiment (FIG. 15B). The long-term anti-tumorigenesis effect of M13-NL in the intestinal tract of IL10^{-/-} mice was tested using colonoscopy. Colorectal tumors were detected in mice in the AOM/PBS and AOM/NL groups from weeks 23 to 30, whereas no colorectal tumors were detected in mice in the AOM/M13-NL group during the same period.

After the mice were sacrificed at week 31, it was found that the colon lengths were significantly increased in the AOM/M13-NL group compared to the other two groups (FIG. 15C). All mice in the AOM/PBS and AOM/NL groups had 4-8 colon tumors (FIG. 15D), whereas 60% of the M13-NL-treated mice (3/5) had no colon tumors, and the remaining 40% (2/5) had only 1-3 colon tumors; thus, the tumor burden was significantly lower in the M13-NL group than in the AOM/PBS and AOM/NL groups (FIG. 15D). Compared with the AOM/M13-NL group, IL10^{-/-} mice in the AOM/PBS and AOM/NL groups showed clear signs of inflammation, including enlarged spleens and significantly higher spleen-to-body weight ratios (FIG. 15E).

### DISCUSSION

The failure to develop a drug candidate stems from its poor biopharmaceutical properties such as low aqueous solubility and/or high chemical instability, metabolic instability, mutagenic potential, and immunogenicity. Although the lead compound 6-shogaol presents potent anti-inflammatory and anti-cancer activities, it has failed to progress to clinical trials due to its poor biopharmaceutical properties, such as high hydrophobicity, poor absorption, high metabolic instability, and concomitant rapid elimination [22]. The tripeptide-conjugated 6-shogaol, M13, is a phase II metabolite of 6-shogaol, which, in the present study, was hypothesized to have better aqueous solubility, chemical stability, and metabolic stability than the lead compound. This study demonstrated that M13 has improved physiochemical properties, including high aqueous solubility and chemical stability, and is not easily ionized at physiological pH. M13 is stable in a mimicked gastric environment (pH=3 or 5) upon incubation at 37° C. for 2 hours, and it has a lower metabolic liability than 6-shogaol. These observations may explain the better anti-inflammatory activities of M13 compared to those of 6-shogaol.

The proportions of circulating CD4⁺ and CD8⁺ T lymphocytes are reportedly increased in IBD patients compared with healthy individuals, and the heightened chronic inflammation in IBD is associated with increased activation of circulating T lymphocytes.[23] M13 seems likely to be safe for healthy individuals, since it does not affect the proportions of resting CD4⁺ or CD8⁺ lymphocytes in vitro. The ability of M13 to inhibit the proliferation of IL-2-activated CD4⁺ and CD8⁺ lymphocytes[24] suggests that M13 may be able to specifically modulate the activated T lymphocytes in IBD patients.

The Ames test, introduced by Bruce Ames in the early 1970s,[25] is widely employed to assess the mutagenic/carcinogenic potential of drug candidates. This test revealed that M13 was not mutagenic up to a concentration of 5000 g/plate. In vitro safety screening is an essential tool for predicting clinical adverse effects during drug discovery, enabling developers to address the possibility of clinical liabilities at an early stage of drug development. The results of our InVEST test demonstrated that M13 did not have any significant effect on the activities of 25 assorted targets from five target classes. In addition, a single-dose oral toxicity study of M13 showed that the maximum tolerated dose of M13 is >1000 mg/kg in mice. These in vitro and in vivo results indicate that M13 can be viewed as a very safe drug candidate. Some early studies showed that M13 exhibited bioactivity in human colon cancer cells (HCT-116) and lung cancer cells (H-1299), while showing low toxicity against human normal colon fibroblast cells (CCD-18Co) and normal lung cells (IMR-90).[14] Here, the growth inhibitory effects of M13 in one human normal intestinal cell line (HIEC-6) and three human intestinal cancer cell lines (HCT-116, Caco-2/BBe, and HC-CSC) were compared. It was found that M13 specifically eliminated intestinal cancer cells (IC50s of 16-40 M) but had much less effect on the growth of normal intestinal cells (IC50>100 ptM). It is known that the natural manner of a solid tumor in vivo can be recapitulated in 3D culture, not 2D culture. Although the 2D cell culture model has been widely used to predict in vivo drug efficacy, it has many limitations, including the lack of tissue-specific structure, cell-to-cell interactions, cell-to-matrix interactions, biochemical cues, and mechanical cues.[26]3D cell culture models in which cells mix and grow within an extracellular matrix gel established by 3D printing are believed to reflect more tissue-specific functions and better mimic the in vivo tumor microenvironment, compared to 2D culture. [27] Here, Caco-2/BBe-hydrogel mixed-cell spheres were 3D printed to simulate the response of a tumor mimic intestinal epithelium to M13 in vivo. Our data showed that M13 treatment is also active and dose-dependent in the 3D Caco-2/BBe sphere model.

Mechanistically, 6-shogaol is thought to act via induction of apoptosis and inhibition of proliferation in cancer.[28] Therefore, it was speculated that M13 (as a tripeptide conjugate of 6-shogaol) might employ similar mechanisms in inhibiting CRC. It was found that 22 cancer-related proteins and two cell cycle control/phosphorylation proteins were significantly down-regulated in Caco-2/BBe cells exposed to M13 or M13-NL. In CRC cells, EGFR is directly involved in regulating Ras/ERK- and PI3K/Akt signaling-mediated apoptosis.[29] The apoptosis-inhibiting proteins, survivin and BCL-x, are regulated by the Notch signaling pathway. [30] Recent studies demonstrated that down-regulation of survivin, EGFR, or BCL-x significantly inhibited tumor growth and induced apoptosis in CRC.[20] In addition, the serum level of Dkk-1 in patients with CRC liver metastasis was reported to be significantly increased, suggesting that down-regulation of Dkk-1 could inhibit CRC liver metastasis. [19] Down-regulation of EpCAM was reported to effectively inhibit the migration and proliferation of CRC cells,[18] while the cyclins CDC25A and RAD52 are essential promoters of CRC cell proliferation.[17] On the basis of these previous findings and the present results, it was concluded that M13 contributes to regulating multiple signaling pathways and various physiological and biochemical reactions, including the apoptosis, proliferation, migration, and metastasis of CRC cells in vitro. This study provides an important reference point for further elucidating the mechanism(s) by which M13 acts against intestinal tumors. Regarding the utilized delivery system, GDNPs offer the advantages of colon-targeting properties, lack of toxicity, low immunogenicity, and ease of mass-production.191 Oral delivery of GDNP-encapsulated M13 has good anti-inflammatory activity in the intestinal tract of mice with UC and can promote intestinal wound healing.[11] The current study demonstrated in vitro that M13-NL was efficiently taken up by cancerous intestinal cells and could down-regulate several cancer-related proteins and cell cycle proteins in Caco-2/BBe cells to a significantly greater degree than free M13, indicating that this encapsulation further enhances the anti-tumor-cell activity of M13. Moreover, it was confirmed that the bioavailability of orally administered NL-M13 in mouse intestinal tissues was significantly increased relative to that of free M13. This action, which may reflect the previously reported ability of NL to target the colon,[11] improved the uptake efficiency of intestinal cells for M13-NL in vivo and increased the concentration and residence time of M13 in colon tissues.

The AOM/DSS-induced CAC mouse model is known as a reproducible and relatively inexpensive initiation-promotion model of CAC that is generated by chemical induction of DNA damage followed by repeated cycles of colitis.[31] This model has been widely used to explore the therapeutic effects of drug candidates on acute inflammation-induced colon cancer.[32] The anti-cancer activities of M13-NL was evaluated in this mouse model, and it was found that orally administered M13-NL could effectively retard the growth of colon tumors in AOM/DSS-induced CAC mice by significantly down-regulating many proteins, including EGFR, survivin, BCL-x, Dkk-1, CDC25A, and RAD52. Furthermore, [10-11] the physiological and biochemical indexes observed in AOM/DSS-induced CAC mice support the biocompatibility of orally administered NL and M13-NL.

It is generally believed that IBD and CAC are related to compositional changes and metabolic dysbiosis of the intestinal microbiota. Accumulating evidence indicates that disturbance of the intestinal microbiota can directly impact the progression of IBD and CAC.[33] NL could target microorganisms and thus significantly accelerate the microbiome composition changes caused by NL-encapsulated 6-shogaol.[34] In the present study, it was found that treatment with M13-NL modulated the gut microbiota by increasing its overall abundance and diversity in AOM/DSS-induced CAC mice. This treatment appeared to enrich phyla with anti-inflammatory functions (e.g., Firmicutes[35]) while depleting phyla with pro-inflammatory functions (e.g., Verrucomicrobia[36]), thereby generating a more balanced microbiota similar to that of healthy mice. This might be another factor contributing to the therapeutic effect of M13-NL on CRC.

The AOM-exposed IL10^{-/-} mouse model, which develops CAC with histopathological features that closely recapitulate IBD-associated colon cancer,[37] is considered a powerful tool for assessing the effects of intestinal microbiota[38] and anti-inflammatory/anti-tumor drug candidates[39] on colon tumorigenesis and CAC development. Here, it was explored whether long-term drinking of water containing PBS supplemented with M13-NL could prevent CRC development in AOM-exposed IL10^{-/-} mice. It was confirmed that long-term oral administration of M13-NL could reduce the spontaneous inflammatory responses in the intestinal tract of IL10^{-/-} mice, improve the composition of the intestinal microbiota to more closely resemble that of low-inflammation IL10^{-/-} mice, and somewhat increase the number of microbial species in intestinal tumors, thereby inhibiting CRC tumorigenesis in AOM-exposed IL10^{-/-} mice without producing severe systemic toxicity. In addition, previous studies showed that spontaneous inflammation in IL10^{-/-} mice can cause splenomegaly,[40] and in the present study described above, it was found that long-term oral administration of M13-NL also could effectively prevent splenomegaly in these mice.

### CONCLUSIONS

In summary, the physicochemical properties, anti-colon cancer activities, and underlying mechanisms of the drug candidate, “M13” were investigated, and preclinical studies of M13-loaded nanoliposomes for the treatment of CRC in vitro and in vivo were conducted. The study shows that M13 has good biopharmaceutical properties, is not mutagenic, and is safe in vitro and in vivo, and that NL encapsulation of M13 improves its delivery efficiency, safety, and effectiveness in vivo. Furthermore, the present work demonstrates that oral delivery of M13-NL has the potential as an effective agent for treating CAC and preventing CAC carcinogenesis.

### REFERENCES

[1]D. Sussman, R. Santaolalla, S. Strobel, R. Dheer, M. T. Abreu, Current opinion in gastroenterology 2012, 28 (4), 327.
[2]S. A. Ingersoll, S. Ayyadurai, M. A. Charania, H. Laroui, Y. Yan, D. Merlin, American Journal of Physiology-Gastrointestinal and Liver Physiology 2012, 302 (5), G484.
[3]D. Long, D. Merlin, Micro- and nanotechnological delivery platforms for treatment of dysbiosis-related inflammatory bowel disease. Future Medicine: 2021; Vol. 16, pp 1741-1745.
[4]A. G. Long, E. T. Lundsmith, K. E. Hamilton, Current colorectal cancer reports 2017, 13 (4), 341.
[5]L. Kotelevets, E. Chastre, D. Desmaele, P. Couvreur, International journal of pharmaceutics 2016, 514 (1), 24.
[6]S. Chen, M.-F. Yueh, C. Bigo, O. Barbier, K. Wang, M. Karin, N. Nguyen, R. H. Tukey, Proceedings of the National Academy of Sciences 2013, 110 (47), 19143.
[7]S. Hua, E. Marks, J. J. Schneider, S. Keely, Nanomedicine: nanotechnology, biology and medicine 2015, 11 (5), 1117.
[8]M. S. Alqahtani, M. Kazi, M. A. Alsenaidy, M. Z. Ahmad, Frontiers in Pharmacology 2021, 62.
[9]C. Yang, M. Zhang, D. Merlin, Journal of Materials Chemistry B 2018, 6 (9), 1312.
[10] M. Zhang, E. Viennois, M. Prasad, Y. Zhang, L. Wang, Z. Zhang, M. K. Han, B. Xiao, C. Xu, S. Srinivasan, Biomaterials 2016, 101, 321.
[11]C. Yang, M. Zhang, S. Lama, L. Wang, D. Merlin, Journal of Controlled Release 2020, 323, 293.
[12]M. Zhang, B. Xiao, H. Wang, M. K. Han, Z. Zhang, E. Viennois, C. Xu, D. Merlin, Molecular Therapy 2016, 24 (10), 1783.
[13]M. Zhang, X. Wang, M. K. Han, J. F. Collins, D. Merlin, Nanomedicine 2017, 12 (16), 1927.
[14]Y. Zhu, R. F. Warin, D. N. Soroka, H. Chen, S. Sang, PLoS one 2013, 8 (1), e54677.
[15]M. Zhang, C. Xu, D. Liu, M. K. Han, L. Wang, D. Merlin, Journal of Crohn's and Colitis 2018, 12 (2), 217.
[16]R. Dahlen, H. Strid, A. Lundgren, S. Isaksson, S. Raghavan, M. K. Magnusson, M. Simren, H. Sjovall, L. Ohman, Scand J Immunol 2013, 78 (3), 275, https://doi.org/10.1111/sji.12081.
[17] a) T. Shen, S. Huang, Anti-Cancer Agents in Medicinal Chemistry (Formerly Current Medicinal Chemistry-Anti-Cancer Agents) 2012, 12 (6), 631; b) J. H. Barlow, R. Rothstein, Cell division 2010, 5 (1), 1.
[18]K.-H. Liang, H.-C. Tso, S.-H. Hung, L L Kuan, J.-K. Lai, F.-Y. Ke, Y.-T. Chuang, L J. Liu, Y.-P. Wang, R.- H. Chen, Cancer letters 2018, 433, 165.
[19] Q. Sui, J. Zheng, D. Liu, J. Peng, Q. Ou, J. Tang, Y. Li, L. Kong, W. Jiang, B. Xiao, BMC cancer 2019, 19 (1), 1.
[20] a) Y. Cai, W. Ma, X. Huang, L. Cao, H. Li, Y. Jiang, N. Lu, Y. Yin, International journal of clinical and experimental pathology 2015, 8 (10), 13267; b) S.-J. Gao, S.-N. Ren, Y.-T. Liu, H.-W. Yan, X.-B. Chen, Molecular Therapy-Oncolytics 2021, 23, 14; c) P. Ramesh, S. Di Franco, L. A. Taboada, L. Zhang, A. Nicotra, G. Stassi, J. P. Medema, Cell Reports 2022, 38 (7), 110374.
[21]B. Chassaing, G. Srinivasan, M. A. Delgado, A. N. Young, A. T. G 2012, e44328.
[22] a) H. Chen, L. Lv, D. Soroka, R. F. Warin, T. A. Parks, Y. Hu, Y. Zhu, X. Chen, S. Sang, Drug Metabolism and Disposition 2012, 40 (4), 742; b) A. Asami, T. Shimada, Y. Mizuhara, T. Asano, S. Takeda, T. Aburada, K.-i. Miyamoto, M. Aburada, Journal of natural medicines 2010, 64 (3), 281.
[23]N. T. Funderburg, S. R. Stubblefield Park, H. C. Sung, G. Hardy, B. Clagett, J. Ignatz-Hoover, C. V. Harding, P. Fu, J. A. Katz, M. M. Lederman, Immunology 2013, 140 (1), 87.
[24]M. F. Bachmann, A. Oxenius, EMBO reports 2007, 8 (12), 1142.
[25]B. N. Ames, J. McCann, E. Yamasaki, Mutat. Res.; (Netherlands) 1975, 31.
[26]F. Pampaloni, E. G. Reynaud, E. H. K. Stelzer, Nature reviews Molecular cell biology 2007, 8 (10), 839.
[27]K. E. Samy, E. S. Levy, K. Phong, B. Demaree, A. R. Abate, T. A. Desai, Scientific reports 2019, 9(1), 1.
[28] a) M. O. Kim, M.-H. Lee, N. Oi, S.-H. Kim, K. B. Bae, Z. Huang, D. J. Kim, K. Reddy, S.-Y. Lee, S. J. Park, Carcinogenesis 2014, 35 (3), 683; b) T. Liang, Y. He, Y. Chang, X. Liu, Biotechnology and Bioprocess Engineering 2019, 24 (3), 560.
[29]Z. Koveitypour, F. Panahi, M. Vakilian, M. Peymani, F. Seyed Forootan, M. H. Nasr Esfahani, K. Ghaedi, Cell & bioscience 2019, 9 (1), 1.
[30]D. T. Rajendran, B. Subramaniyan, M. Ganeshan, in Springer, 2017.
[31] a) T. Tanaka, H. Kohno, R. Suzuki, Y. Yamada, S. Sugie, H. Mori, 2003, 94 (11), 965; b) M. De Robertis, E. Massi, M. L. Poeta, S. Carotti, S. Morini, L. Cecchetelli, E. Signori, V. M. Fazio, Journal of carcinogenesis 2011, 10.
[32] a) Y. Chen, B. Wang, X. Yuan, Y. Lu, J. Hu, J. Gao, J. Lin, J. Liang, S. Hou, S. Chen, Phytomedicine 2021, 83, 153489; b) J. Osman, S. Savari, N. K. Chandrashekar, K. Bellamkonda, D. Douglas, A. Sjölander, Oncotarget 2017, 8 (21), 34773.
[33] a) S. H. Wong, J. Yu, Nature Reviews Gastroenterology & Hepatology 2019, 16 (11), 690; b) I. Khan, N. Ullah, L. Zha, Y. Bai, A. Khan, T. Zhao, T. Che, C. Zhang, Pathogens 2019, 8 (3), 126.
[34]C. Yang, D. Long, J. Sung, Z. Alghoul, D. Merlin, Pharmaceutics 2021, 13 (9), 1355.
[35]R. B. Sartor, S. K. Mazmanian, The American journal of gastroenterology supplements 2012, 1(1), 15.
[36]C.-H. Lin, C.-C. Chen, H.-L. Chiang, J.-M. Liou, C.-M. Chang, T.-P. Lu, E. Y. Chuang, Y.-C. Tai, C. Cheng, H.-Y. Lin, Journal of neuroinflammation 2019, 16 (1), 1.
[37]A. Rothemich, J. C. Arthur, in Springer, 2019.
[38]J. M. Uronis, M. MUhlbauer, H. H. Herfarth, T. C. Rubinas, G. S. Jones, C. Jobin, PloS one 2009, 4 (6), e6026.
[39]R.-M. T. McFadden, C. B. Larmonier, K. W. Shehab, M. Midura-Kiela, R. Ramalingam, C. A. Harrison, D. G. Besselsen, J. H. Chase, J. G. Caporaso, C. Jobin, Inflammatory bowel diseases 2015, 21(11), 2483.
[40] a) D. C. Gunasekera, J. Ma, V. Vacharathit, P. Shah, A. Ramakrishnan, P. Uprety, Z. Shen, A. Sheh, C. F. Brayton, M. T. Whary, Mucosal immunology 2020, 13 (3), 493; b) D. C. Ranatunga, A. Ramakrishnan, P. Uprety, F. Wang, H. Zhang, J. B. Margolick, C. Brayton, J. H. Bream, The Journal of Immunology 2012, 189 (3), 1243.
[41] Salminen, A., Kauppinen, A., Suuronen, T. and Kaarniranta, K. Bioessays 2008, 30: 939-942.
[42] Baltimore D. Discovering NF-kappaB. Cold Spring Harb Perspect Biol. 2009 July; 1(1): a000026.

Those skilled in the art will recognize, or be able to ascertain using no more than routine experimentation, many equivalents to the specific embodiments of the method and compositions described herein. Such equivalents are intended to be encompassed by the following claims.

1. A compound having the structure: 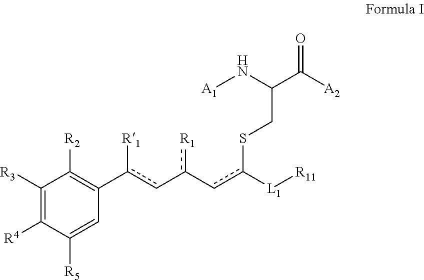
wherein:
(i) [symbol] is a single or double bond;
(ii) A₁ is an amino acid residue wherein the amino acid forming A₁ has a pH similar to glutamic acid;
(iii) A₂ is an amino acid residue wherein the amino acid forming A₂ has a pH similar to glycine;
(iv) R₁ is an oxygen, a hydroxyl, or —OR₆, and R₆ is an unsubstituted linear, branched, or cyclol C₁-C₆ alkyl;
(v) R′₁ is a hydrogen, a hydroxyl, or —OR₈, and R₈ is an unsubstituted linear, branched, or cyclol C₁-C₆ alkyl;
(vi) R₂-R₅ are independently a hydrogen, a hydroxyl, a halogen, an haloalkyl (e.g., —CF₃), or —OR₇, R₇ is an unsubstituted linear, branched, or cyclol C₁-C₆ alkyl, —C(═O)R₉, R₉ is hydrogen, an unsubstituted linear, branched, or cyclol C₁-C₆ alkyl, or —OR₁₀, and R₁₀ is hydrogen or an unsubstituted linear, branched, or cyclol C₁-C₆ alkyl;
(vii) L₁ is a single bond or —(CH₂)ₘ—, m is an integer from 1 to 8;
(viii) R₁₁ is a hydrogen, an unsubstituted or substituted linear C₁-C₁₀ alkyl, an unsubstituted or substituted branched C₃-C₁₀ alkyl, an unsubstituted or substituted C₃-C₁₀ cycloalkyl, or an unsubstituted or substituted C₅-C₁₂ aryl; and
(ix) each substituent, when present, is independently a halogen, a hydroxyl, an haloalkyl, an unsubstituted linear, branched, or cyclol C₁-C₆ alkyl, an unsubstituted C₅-C₁₂ aryl, or —OR₁₂, R₁₂ is an unsubstituted linear, branched, or cyclol C₁-C₆ alkyl, and wherein the compound is not:

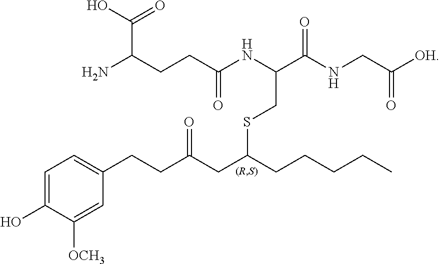 [SMILES: CCCCCC(CC(=O)CCc1ccc(O)c(OC)c1)SCC(NC(=O)CCC(N)C(=O)O)C(=O)NCC(=O)O]

2. The compound of claim 1, wherein the compound has the structure: 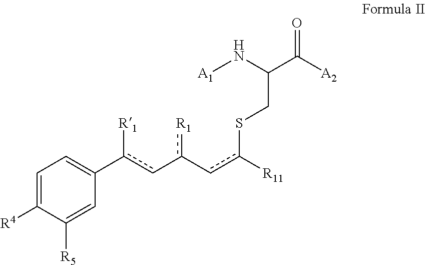
wherein:
(i) [symbol] is a single or double bond;
(ii) A₁ is an amino acid residue wherein the amino acid forming A₁ has a pH similar to glutamic acid;
(iii) A₂ is an amino acid residue wherein the amino acid forming A₂ has a pH similar to glycine;
(iv) R₁ is an oxygen, a hydroxyl, or —OR₆, and R₆ is an unsubstituted linear, branched, or cyclol C₁-C₆ alkyl;
(v) R′₁ is a hydrogen, a hydroxyl, or —OR₈, and R₈ is an unsubstituted linear, branched, or cyclol C₁-C₆ alkyl;
(vi) R₄ and R₅ are independently a hydrogen, a hydroxyl, or —OR₇, R₇ is an unsubstituted linear, branched, or cyclol C₁-C₆ alkyl; and
(vii) R₁₁ is an unsubstituted linear C₅-C₉ alkyl, an unsubstituted branched C₃-C₈ alkyl, an unsubstituted C₃-C₈ cycloalkyl, an unsubstituted C₅-C₁₂ aryl, or a C₅-C₁₂ aryl substituted with a halogen, a hydroxyl, a haloalkyl, or —OR₁₂, R₁₂ is an unsubstituted linear, branched, or cyclol C₁-C₆ alkyl, or a combination thereof.

3. The compound of claim 1, wherein the compound has the structure: 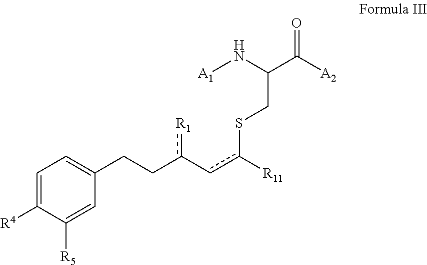
wherein:
(i) [symbol] is a single or double bond;
(ii) A₁ is an amino acid residue wherein the amino acid forming A₁ has a pH similar to glutamic acid;
(iii) A₂ is an amino acid residue wherein the amino acid forming A₂ has a pH similar to glycine;
(iv) R₁ is oxygen, hydroxyl, or —OR₆, and R₆ is an unsubstituted C₁-C₆ alkyl;
(v) R₄ is a hydrogen or hydroxyl;
(vi) R₅ is a hydrogen, a hydroxyl, or —OR₇, and R₇ is an unsubstituted C₁-C₆ alkyl; and
(vii) R₁₁ is an unsubstituted branched C₃-C₆ alkyl, an unsubstituted C₃-C₆ cycloalkyl, an unsubstituted phenyl, or a phenyl substituted with a halogen.

4. The compound of claim 1, wherein the compound has the structure: 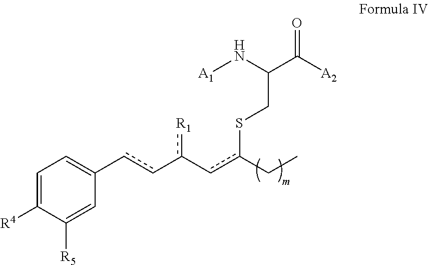
wherein:
(i) [symbol] is a single or double bond;
(ii) m is an integer from 4 to 8;
(iii) A₁ is an amino acid residue wherein the amino acid forming A₁ has a pH similar to glutamic acid;
(iv) A₂ is an amino acid residue wherein the amino acid forming A₂ has a pH similar to glycine;
(v) R₁ is oxygen, hydroxyl, or —OR₆, and R₆ is an unsubstituted linear, branched, or cyclol C₁-C₆ alkyl;
(vi) R₄ is a hydrogen or hydroxyl; and
(vii) R₅ is a hydrogen, a hydroxyl, or —OR₇, and R₇ is an unsubstituted linear, branched, or cyclol C₁-C₆ alkyl.

5. The compound of claim 4, wherein the compound has the structure: 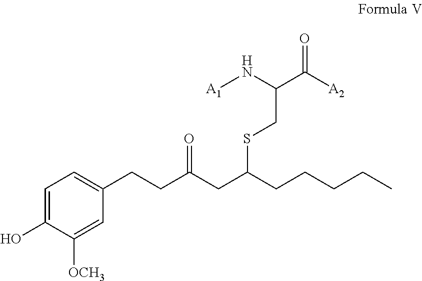
wherein A₁ is an amino acid residue wherein the amino acid forming A₁ has a pH similar to glutamic acid; and A₂ is an amino acid residue wherein the amino acid forming A₂ has a pH similar to glycine.

6. The compound of claim 1, wherein A₁ is a glutamic acid residue or an aspartic acid residue, or a synthetic derivative thereof, optionally an alpha-glutamic acid residue, a gamma-glutamic acid residue, an alpha-aspartic acid residue, or a beta-aspartic acid residue; and wherein A₂ is an alanine residue, a phenylalanine residue, a leucine residue, a methionine residue, a proline residue, a valine residue, a tryptophan residue, a tyrosine residue, or a glycine residue, or a synthetic derivative thereof.

7. The compound of claim 1, wherein the compound has the structure:

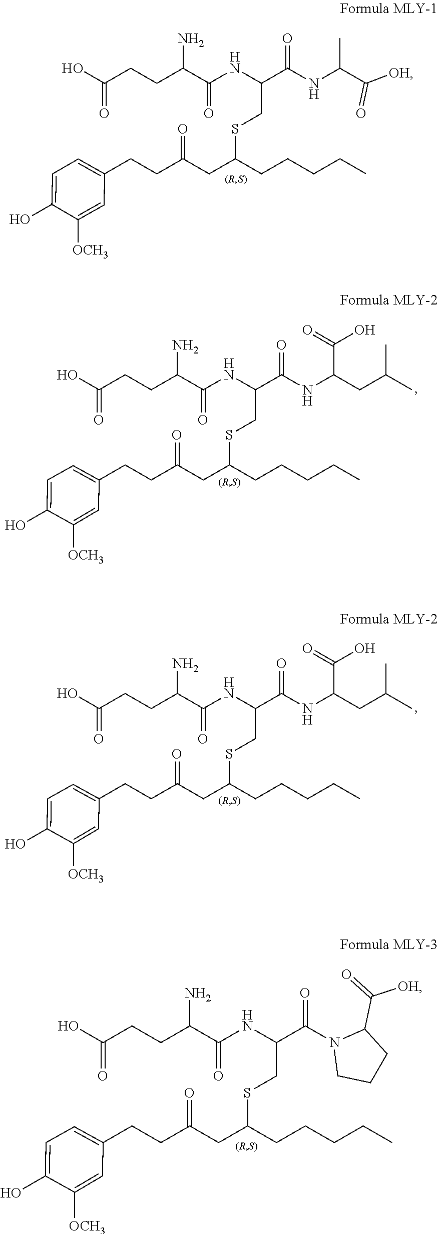 [SMILES: CCCCCC(CC(=O)CCc1ccc(O)c(OC)c1)SCC(NC(=O)C(N)CCC(=O)O)C(=O)NC(C)C(=O)O ; CCCCCC(CC(=O)CCc1ccc(O)c(OC)c1)SCC(NC(=O)C(N)CCC(=O)O)C(=O)NC(CC(C)C)C(=O)O ; CCCCCC(CC(=O)CCc1ccc(O)c(OC)c1)SCC(NC(=O)C(N)CCC(=O)O)C(=O)NC(CC(C)C)C(=O)O ; CCCCCC(CC(=O)CCc1ccc(O)c(OC)c1)SCC(NC(=O)C(N)CCC(=O)O)C(=O)N1CCCC1C(=O)O]

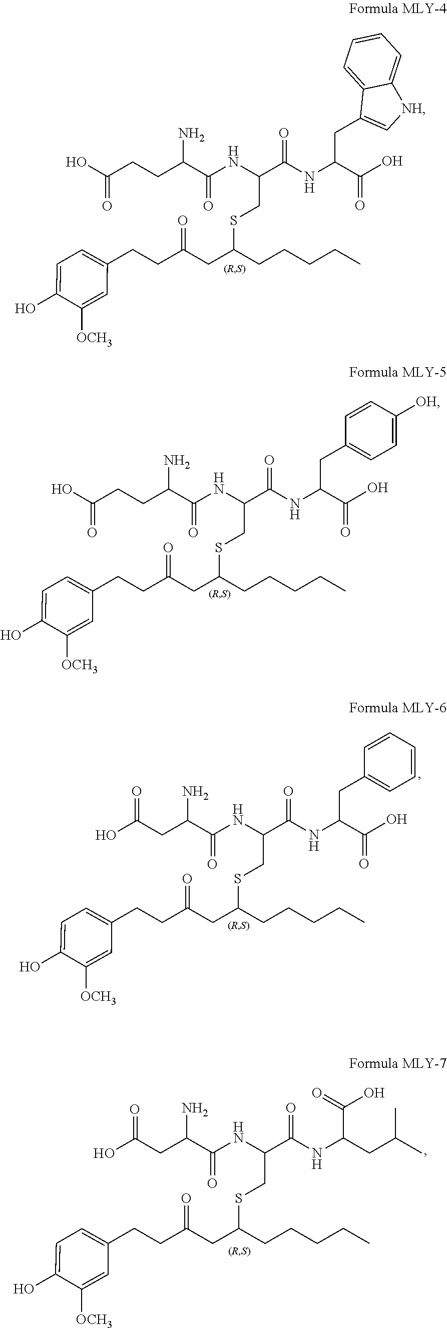 [SMILES: CCCCCC(CC(=O)CCc1ccc(O)c(OC)c1)SCC(NC(=O)C(N)CCC(=O)O)C(=O)NC(Cc1c[nH]c2ccccc12)C(=O)O ; CCCCCC(CC(=O)CCc1ccc(O)c(OC)c1)SCC(NC(=O)C(N)CCC(=O)O)C(=O)NC(Cc1ccc(O)cc1)C(=O)O ; CCCCCC(CC(=O)CCc1ccc(O)c(OC)c1)SCC(NC(=O)C(N)CC(=O)O)C(=O)NC(Cc1ccccc1)C(=O)O ; CCCCCC(CC(=O)CCc1ccc(O)c(OC)c1)SCC(NC(=O)C(N)CC(=O)O)C(=O)NC(CC(C)C)C(=O)O]

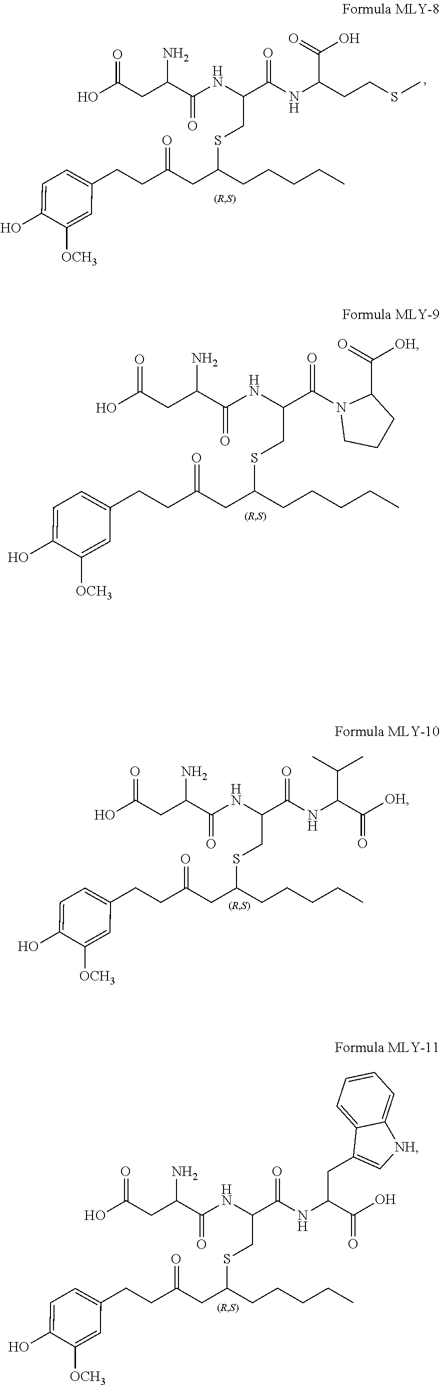 [SMILES: CCCCCC(CC(=O)CCc1ccc(O)c(OC)c1)SCC(NC(=O)C(N)CC(=O)O)C(=O)NC(CCSC)C(=O)O ; CCCCCC(CC(=O)CCc1ccc(O)c(OC)c1)SCC(NC(=O)C(N)CC(=O)O)C(=O)N1CCCC1C(=O)O ; CCCCCC(CC(=O)CCc1ccc(O)c(OC)c1)SCC(NC(=O)C(N)CC(=O)O)C(=O)NC(C(=O)O)C(C)C ; CCCCCC(CC(=O)CCc1ccc(O)c(OC)c1)SCC(NC(=O)C(N)CC(=O)O)C(=O)NC(Cc1c[nH]c2ccccc12)C(=O)O]

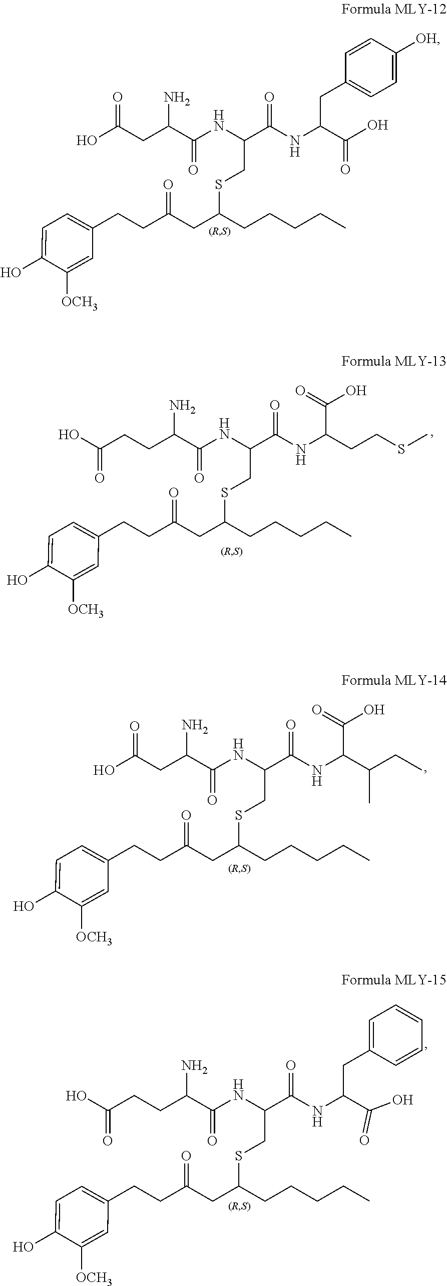 [SMILES: CCCCCC(CC(=O)CCc1ccc(O)c(OC)c1)SCC(NC(=O)C(N)CC(=O)O)C(=O)NC(Cc1ccc(O)cc1)C(=O)O ; CCCCCC(CC(=O)CCc1ccc(O)c(OC)c1)SCC(NC(=O)C(N)CCC(=O)O)C(=O)NC(CCSC)C(=O)O ; CCCCCC(CC(=O)CCc1ccc(O)c(OC)c1)SCC(NC(=O)C(N)CC(=O)O)C(=O)NC(C(=O)O)C(C)CC ; CCCCCC(CC(=O)CCc1ccc(O)c(OC)c1)SCC(NC(=O)C(N)CCC(=O)O)C(=O)NC(Cc1ccccc1)C(=O)O]

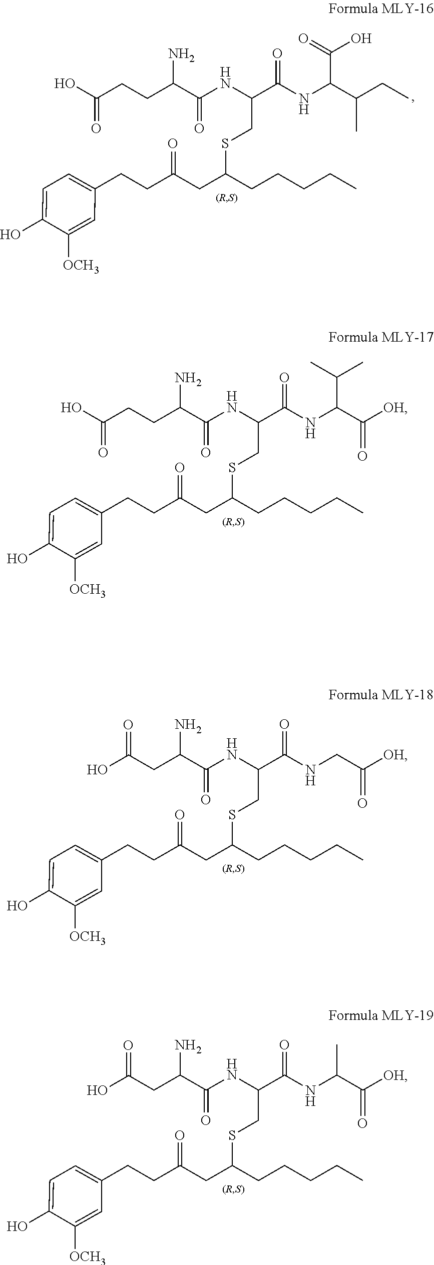 [SMILES: CCCCCC(CC(=O)CCc1ccc(O)c(OC)c1)SCC(NC(=O)C(N)CCC(=O)O)C(=O)NC(C(=O)O)C(C)CC ; CCCCCC(CC(=O)CCc1ccc(O)c(OC)c1)SCC(NC(=O)C(N)CCC(=O)O)C(=O)NC(C(=O)O)C(C)C ; CCCCCC(CC(=O)CCc1ccc(O)c(OC)c1)SCC(NC(=O)C(N)CC(=O)O)C(=O)NCC(=O)O ; CCCCCC(CC(=O)CCc1ccc(O)c(OC)c1)SCC(NC(=O)C(N)CC(=O)O)C(=O)NC(C)C(=O)O]

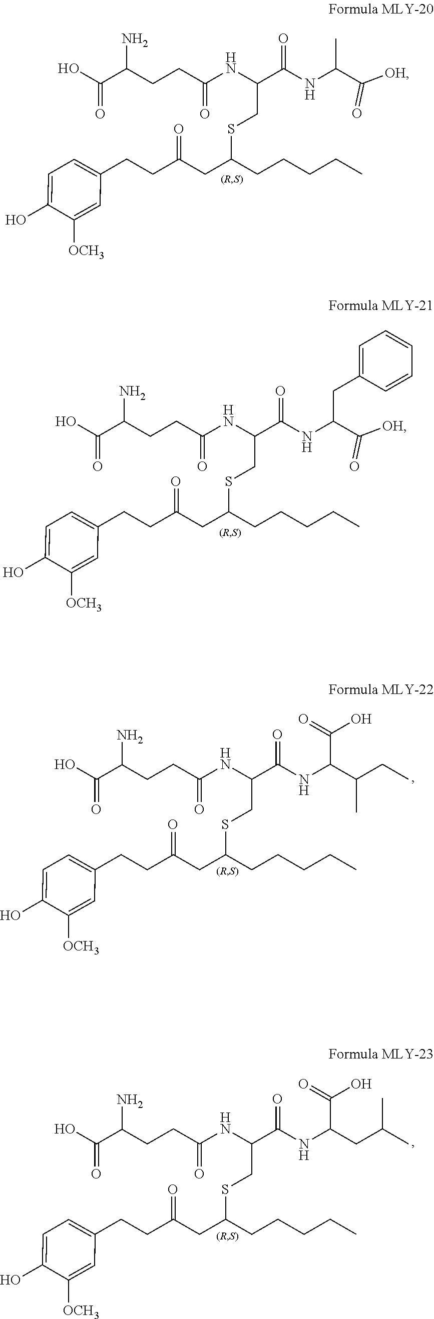 [SMILES: CCCCCC(CC(=O)CCc1ccc(O)c(OC)c1)SCC(NC(=O)CCC(N)C(=O)O)C(=O)NC(C)C(=O)O ; CCCCCC(CC(=O)CCc1ccc(O)c(OC)c1)SCC(NC(=O)CCC(N)C(=O)O)C(=O)NC(Cc1ccccc1)C(=O)O ; CCCCCC(CC(=O)CCc1ccc(O)c(OC)c1)SCC(NC(=O)CCC(N)C(=O)O)C(=O)NC(C(=O)O)C(C)CC ; CCCCCC(CC(=O)CCc1ccc(O)c(OC)c1)SCC(NC(=O)CCC(N)C(=O)O)C(=O)NC(CC(C)C)C(=O)O]

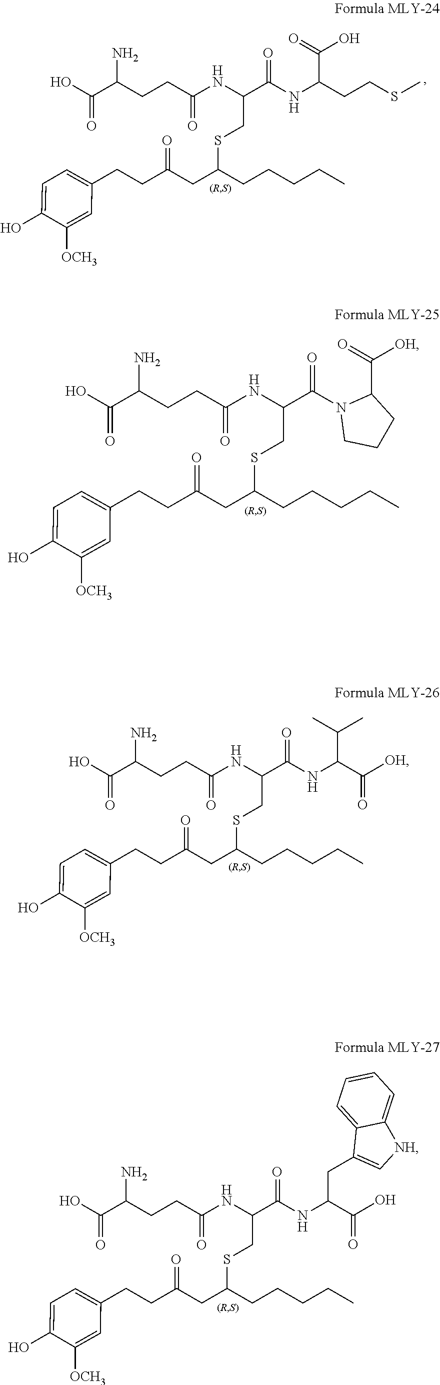 [SMILES: CCCCCC(CC(=O)CCc1ccc(O)c(OC)c1)SCC(NC(=O)CCC(N)C(=O)O)C(=O)NC(CCSC)C(=O)O ; CCCCCC(CC(=O)CCc1ccc(O)c(OC)c1)SCC(NC(=O)CCC(N)C(=O)O)C(=O)N1CCCC1C(=O)O ; CCCCCC(CC(=O)CCc1ccc(O)c(OC)c1)SCC(NC(=O)CCC(N)C(=O)O)C(=O)NC(C(=O)O)C(C)C ; CCCCCC(CC(=O)CCc1ccc(O)c(OC)c1)SCC(NC(=O)CCC(N)C(=O)O)C(=O)NC(Cc1c[nH]c2ccccc12)C(=O)O]

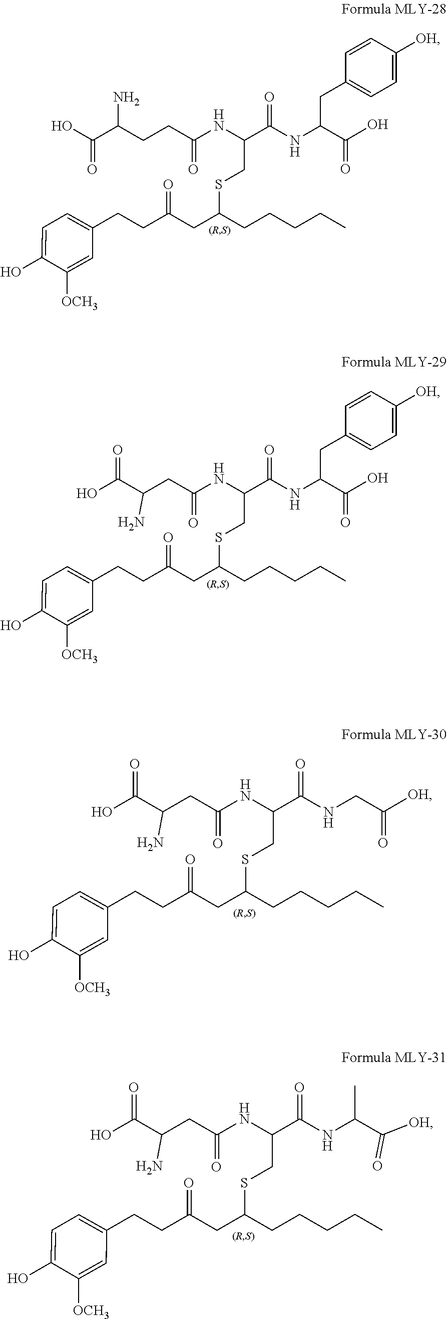 [SMILES: CCCCCC(CC(=O)CCc1ccc(O)c(OC)c1)SCC(NC(=O)CCC(N)C(=O)O)C(=O)NC(Cc1ccc(O)cc1)C(=O)O ; CCCCCC(CC(=O)CCc1ccc(O)c(OC)c1)SCC(NC(=O)CC(N)C(=O)O)C(=O)NC(Cc1ccc(O)cc1)C(=O)O ; CCCCCC(CC(=O)CCc1ccc(O)c(OC)c1)SCC(NC(=O)CC(N)C(=O)O)C(=O)NCC(=O)O ; CCCCCC(CC(=O)CCc1ccc(O)c(OC)c1)SCC(NC(=O)CC(N)C(=O)O)C(=O)NC(C)C(=O)O]

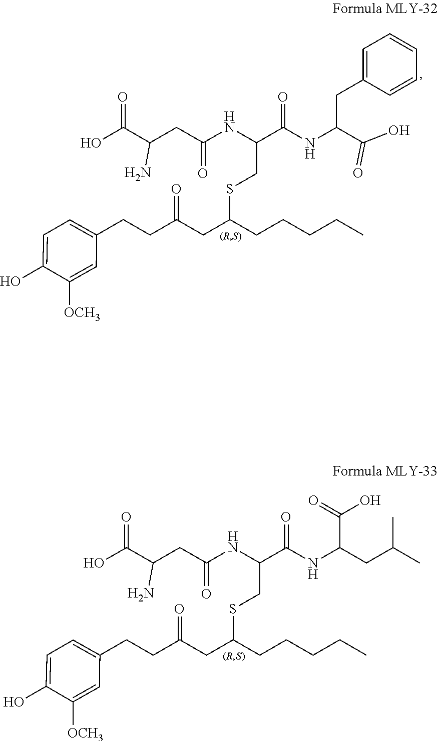 [SMILES: CCCCCC(CC(=O)CCc1ccc(O)c(OC)c1)SCC(NC(=O)CC(N)C(=O)O)C(=O)NC(Cc1ccccc1)C(=O)O ; CCCCCC(CC(=O)CCc1ccc(O)c(OC)c1)SCC(NC(=O)CC(N)C(=O)O)C(=O)NC(CC(C)C)C(=O)O]

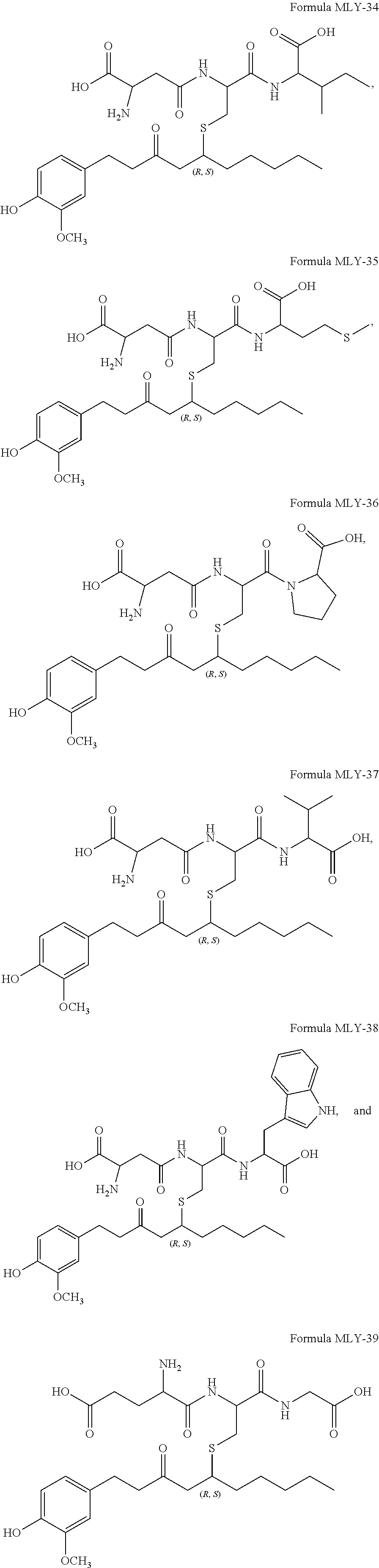 [SMILES: CCCCCC(CC(=O)CCc1ccc(O)c(OC)c1)SCC(NC(=O)CC(N)C(=O)O)C(=O)NC(C(=O)O)C(C)CC ; CCCCCC(CC(=O)CCc1ccc(O)c(OC)c1)SCC(NC(=O)CC(N)C(=O)O)C(=O)NC(CCSC)C(=O)O ; CCCCCC(CC(=O)CCc1ccc(O)c(OC)c1)SCC(NC(=O)CC(N)C(=O)O)C(=O)N1CCCC1C(=O)O ; CCCCCC(CC(=O)CCc1ccc(O)c(OC)c1)SCC(NC(=O)CC(N)C(=O)O)C(=O)NC(C(=O)O)C(C)C ; CCCCCC(CC(=O)CCc1ccc(O)c(OC)c1)SCC(NC(=O)CC(N)C(=O)O)C(=O)NC(Cc1c[nH]c2ccccc12)C(=O)O ; CCCCCC(CC(=O)CCc1ccc(O)c(OC)c1)SCC(NC(=O)C(N)CCC(=O)O)C(=O)NCC(=O)O]

8. A pharmaceutical formulation comprising one or more compounds of claim 1 or M13, and one or more pharmaceutically acceptable carriers and/or excipients.

9. The pharmaceutical formulation of claim 8, wherein the one or more compounds in the formulation are collectively or each individually in an amount effective, when administered to a subject, to:
reduce the expression in the subject of one or more pro-inflammatory cytokines and/or chemokines selected from the group consisting of TNFα, 1L6, and IL1-β,
increase the expression of IL-10 in the subject,
reduce the expression in the subject of one or more kinases selected from the group consisting of PI3K, SPHK2, CDK6, TRKB, C-MER, EPHB2, RIPK4, RIPK5, and RIPK4, or a combination thereof.

10-18. (canceled)

19. The pharmaceutical formulation of claim 8, wherein the one or more compounds in the formulation are collectively or each individually in an amount effective, when administered to a subject, to treat or to ameliorate one or more symptoms of a disease associated with the viral infection, the microbial infection, the cancer, the inflammatory response, or a combination thereof, in the subject.

20. (canceled)

21. The pharmaceutical formulation of claim 8, further comprising one or more additional active agents selected from the group consisting of a therapeutic agent, a prophylactic agent, a diagnostic agent, an anti-inflammatory agent, and an adjuvant.

22. (canceled)

23. The pharmaceutical formulation of claim 8, wherein the one or more compounds in the pharmaceutical formulation are collectively at a concentration of at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 Wt %.

24. The pharmaceutical formulation of claim 8, wherein the one or more compounds in the pharmaceutical formulation are each individually at a concentration of at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 Wt %.

25. A method of treating a subject, the method comprising:
administering to the subject the pharmaceutical formulation of claim 8, wherein the one of more compounds in the formulation are collectively in an amount effective to treat a viral infection, a microbial infection, cancer, an inflammatory response, or a combination thereof, in the subject,
wherein the administering is performed one or more times.

26. The method of claim 25, wherein the one or more compounds in the formulation are:
each individually in an amount effective to treat a viral infection, a microbial infection, cancer, an inflammatory response, a disease associated with a viral infection, a microbial infection, cancer, an inflammatory response, or a combination thereof, in the subject; or
collectively in an amount effective to ameliorate one or more symptoms of the viral infection, the microbial infection, the cancer, the inflammatory response, a disease associated with a viral infection, a microbial infection, a cancer, an inflammatory response, or a combination thereof, in the subject.

27-30. (canceled)

31. The method of claim 25, wherein the subject has a viral infection.

32. The method of claim 25, wherein the viral infection is an infection by an RNA virus, optionally wherein the RNA virus is of a family selected from the group consisting of Flaviviridae, Orthomyxoviridae, Filoviridae, Coronaviridae, and Paramyxoviridae,
optionally wherein the viral infection is an infection by a coronavirus,
optionally wherein the coronavirus is an alpha coronavirus, a beta coronavirus, a gamma coronavirus, or a delta coronavirus,
optionally wherein the coronavirus is selected from the group consisting of Human Coronavirus 229E (HCoV-229E), Human Coronavirus OC43 (HCoV-OC43), Human Coronavirus NL63 (HCoV-NL63), Human Coronavirus HKU1 (HCoV-HKU1), Middle East Respiratory Syndrome Coronavirus (MERS-CoV), Severe Acute Respiratory Syndrome Coronavirus 1 (SARS-CoV-1), and SARS-CoV-2.

33-38. (canceled)

39. The method of claim 25, wherein the subject has a microbial infection,
optionally wherein the microbial infection is caused by a microorganisms selected from the group consisting of a bacterium, a virus, a protozoan, and a fungus.

40. (canceled)

41. The method of claim 25, wherein the subject has cancer or an inflammatory condition.

42-71. (canceled)
